(19) United States
(12) Patent Application Publication
(10) Pub. No.: US 2012/0003655 A1
(43) Pub. Date: Jan. 5, 2012

(54) IDENTIFICATION OF NEUROPROTECTIVE AGENTS USING PRO-INFLAMMATORY HUMAN GLIAL CELLS

(75) Inventors: Maria C.N. Marchetto, La Jolla, CA (US); Fred H. Gage, La Jolla, CA (US); Christopher K. Glass, San Diego, CA (US); Kaoru Saijo, San Diego, CA (US); Beate Winner, San Diego, CA (US)

(73) Assignee: The Salk Institute for Biological Studies, La Jolla, CA (US)

(21) Appl. No.: 13/132,260

(22) PCT Filed: Dec. 3, 2009
(86) PCT No.: PCT/US09/66641

§ 371 Date: Sep. 19, 2011

Related U.S. Application Data
(60) Provisional application No. 61/119,700, filed on Dec. 3, 2008.

(51) Int. Cl. C12Q 1/02; C12Q 1/66; C12Q 1/68; C12Q 1/26; G01N 21/64

(52) U.S. Cl. 435/6.12; 435/29; 435/25; 435/7.1; 435/7.4; 435/6.13; 435/8

(57) ABSTRACT

Provided herein are methods for, inter alia, identifying new therapeutic agents using human cell-based models.

### CROSS-REFERENCES TO RELATED APPLICATIONS

This patent application is a National Stage of PCT/US2009/066641, filed Dec. 3, 2009, and claims the benefit of Unites Stated Provisional Patent Application No. 61/119,700, filed Dec. 3, 2008, the contents of which are hereby incorporated by reference in their entireties and for all purposes.

### STATEMENT AS TO RIGHTS TO INVENTIONS MADE UNDER FEDERALLY SPONSORED RESEARCH OR DEVELOPMENT

This invention was made with government support under CA52599 awarded by the National Institutes of Health. The Government has certain rights in the invention.

### BACKGROUND OF THE INVENTION

Neurological disorders affect large portions of the human population each year. For example, amyotrophic lateral sclerosis (ALS) is a progressive neurodegenerative adult disease characterized by fatal paralysis in both the brain and spinal cord motor neurons. Parkinson's disease (PD) is the most prevalent movement disorder among people over 65 years old. The denervation of dopaminergic neurons in the substantia nigra (SN) results in severely debilitating motor symptoms such as bradykinesia, resting tremor and rigidity (Farrer, 2006; Fearnley and Lees, 1991). Currently, there are very few neuroprotective agents that effectively treat these disorders. For example, there is only one FDA-approved treatment for ALS, namely riluzole (Doble, 1996), and it only extends the course of the disease for 2 months (Miller and Moore, 2004).

Therefore, there is an urgent need for additional and improved treatments for neurological disorders such as ALS and PD. The methods provided herein solve these and other needs in the art.

### BRIEF SUMMARY OF THE INVENTION

Provided herein are methods for, inter alia, identifying new therapeutic agents using human cell-based models. In particular, pro-inflammatory human glial cells may be used in rapid drug screening tests. In addition, human co-culture models using pro-inflammatory human glial cells and human neuronal cells are also provided. Previous murine models have shown inefficacy in both pre-clinical and clinical human trials (DiBernardo and Cudkowicz, 2006; Scott et al., 2008). Therefore, the use of human co-culture models will critically impact the unveiling of complex metabolic pathways involved in neurological diseases.

In one aspect, a method is provided for determining whether a test agent is a neuroprotective agent. The method includes adding a test agent to a cellular culture. The cellular culture includes pro-inflammatory human glial cells. A level of pro-inflammatory activity of the pro-inflammatory human glial cells is determined in the presence of the test agent. The level of pro-inflammatory activity of the pro-inflammatory human glial cells in the presence of the test agent is compared to a control thereby determining whether the test agent is a neuroprotective agent. In some embodiments, a lower level of pro-inflammatory activity of the pro-inflammatory human glial cells in the presence of the test agent compared to a control is indicative of the test agent being a neuroprotective agent.

In another aspect, a method is provided for determining whether a test agent is a neuroprotective agent. The method includes adding a test agent to a cellular culture comprising pro-inflammatory human astrocyte cells. A level of pro-inflammatory activity of the pro-inflammatory human astrocyte cells in the presence of the test agent is determined. The level of pro-inflammatory activity of the pro-inflammatory human astrocyte cells in the presence of the test agent is compared to a control thereby determining whether the test agent is a neuroprotective agent.

In another aspect, a method is provided for treating a disease mediated by a human glial cell inflammatory response in a human subject in need thereof. The method includes administering to the human subject an effective amount of an anti-inflammatory agent.

### BRIEF DESCRIPTION OF THE DRAWINGS

FIGS. 1A-R show differentiation and functional characterization of HESC-derived motor neurons. FIGS. 1A-1D depict neuroectodermal rosettes expressing motor neuron-progenitor markers, Pax6, Nestin, Olig2 and Islet1, after 2-3 weeks of differentiation. FIGS. 1E-1G depict that expression of motor neuron postmitotic markers Hb9, HoxC8 and ChAT was detected after 4 weeks of differentiation. FIG. 1H depicts RT-PCR of HESC-derived motor neurons showing down-regulation of HESC marker, Nanog, and confirming the expression of motor neuron subtype markers such as Hb9 and ChAT. FIG. 1I depicts synapsin-expressing neurites and FIG. 1J depicts α-Bungarotoxin incorporation at neuromuscular junctions following co-culture with C2C12 myoblasts observed after 7-8 weeks of differentiation. FIG. 1K depicts expression of endogenous Hb9 co-localizing with Hb9::GFP-positive cells in human motor neurons. FIG. 1L depicts co-localization between ChAT-positive (inset) and Hb9::GFP motor neuron. FIG. 1M depicts a fluorescence micrograph of the Hb9-positive cell from which data shown in (FIGS. 1N-1R) were obtained. FIG. 1N depicts transient Na⁺ and sustained K⁺ currents (upper panel, the asterisk and arrow indicate Na⁺ and K⁺ currents, respectively) in response to step depolarizations (lower panel; cell voltage-clamped at −70 mV, command voltage from −90 to +100 mV, 10 mV step). FIGS. 1O-1P depict I-V relations corresponding to peak Na⁺ currents (FIG. 1O) and steady state K⁺ currents. (FIG. 1Q) Sub- and supra-threshold responses (upper panel) to somatic current injections (lower panel: cell current-clamped at around −80 mV, currents from 10 to 30 pA, 10 pA step). FIG. 1R depicts spontaneous action potentials when the cell was current-clamped at −60 mV. Scale bars: FIGS. 1A-1F, 100 μm; FIG. 1G, 80 μm; FIG. 1I, 20 μm and FIGS. 1J-1M, 40 μm.

FIGS. 2A-D show HESC-derived neuronal co-cultures with human astrocytes. (FIG. 2A) Experimental design: Human primary astrocytes were infected with LentiSOD1^{WT} or LentiSOD1^{G37R} for SOD1 (wild type or mutated) overexpression. HESCs were differentiated into motor neuron precursors (rosettes), gently dissociated and plated on two different glial monolayers. The co-cultures were then infected with LentiHb9::GFP and carried out for 3 more weeks. The motor neurons were detected by GFP fluorescent sorting (FACS) or ChAT immunofluorescence. (FIG. 2B) Hb9::GFP-positive neurons co-cultured with Astro SOD1^{WT} or Astro SOD1^{G37R} and corresponding GFP fluorescence quantification by FACS. (FIG. 2C) Astrocytes co-cultures overexpressing either SOD1^{WT} or SOD1^{G37R} and quantification of cholinergic motor neurons. (FIG. 2D) Representative fields of GABAergic neurons detected by glutamic acid decarboxylase 65 (GAD65) immunoreactivity present in the co-cultures concomitantly with the motor neurons and corresponding quantification. Scale bars: 80 μm.

FIGS. 3A-D show Inflammatory response in astrocytes expressing mutated SOD1. (FIG. 3A) Astrocytes' reactivity, measured by expression of glial fibrillary acidic protein (GFAP) in control (Astro SOD1^{WT}) versus mutated (AstroSOD1^{G37R}) astrocytes. Note that the expression of A2B5, a marker that is not related to astrocytic immune response, is the same in both conditions. (FIG. 3B) Quantification of the production of reactive species of oxygen (ROS) in control versus SOD1^{G37R} astrocytes. Graphs show percentage of cells producing ROS and fluorescence intensity. (FIG. 3C) Western blot showing differential expression of inducible nitric oxide synthase (iNOS), the gp91phox (NOX 2) subunit from NADPH oxidase, secretory proteins chromogranin A (CHGA) and cystatin C(CC) in control versus mutated astrocytes. (FIG. 3D) Indirect measure of nitric oxide by Griess method in astrocytes conditioned media. Scale bar: 80 μM.

FIGS. 4A-C show screening of compounds and their ability to decrease oxidation in SOD1^{G37R} astrocytes. (FIG. 4A) Detection of ROS production in SOD^{G37R} astrocytes. Green fluorescence was used to mark cells that undergo oxidation. (FIG. 4B) Quantification of the number of cells producing ROS. (FIG. 4C) Relative intensity of fluorescence. Scale bar: 80 μm.

FIGS. 5A-B show recovery of motor neuron survival after treatment with apocynin. (FIG. 5A) Immunofluorescence of representative images from co-cultures of HESC-derived motor neurons and SOD^{wt} or SOD^{G37R} astrocytes that were treated with apocynin or vehicle. (FIG. 5B) Quantification of ChAT-positive cells in the different conditions. Scale bar: 80 μm.

FIGS. 6A-B show drug screening schematics using human astrocytes and HESC-derived motor neurons. (FIG. 6A) HESC were differentiated into neuronal rosettes that were further maturated into electrophysiologically functional cells expressing typical motor neuron markers. (FIG. 6B) Astrocytes were treated with a number of compounds (ex. anti-oxidant drugs/flavonoids) and tested for either oxidation levels or motor neuron survival rate upon co-culture.

FIGS. 7A-C show validation of human astrocytes as ALS model after SOD^{WT} or SOD^{G37R} Lentivirus infection. (FIG. 7A) Quantification of Lenti-SOD1^{G37R} viral particles using immunofluorescence. Virus tittering was performed in rat neural stem cells (NSC) using an antibody that specifically recognizes human SOD1 protein. (FIG. 7B) Ectopic expression of SOD^{WT} or SOD^{G37R} in human astrocytes after lentiviral infection. (FIG. 7C) Cell death quantification by propidium iodide (PI) staining Scale bar: 200 μm.

FIGS. 8A-D show specificity of Lenti-Hb9::GFP virus. Co-localization of LentiHb9::GFP expression with endogenous Hb9 protein in (FIG. 8A) purified primary rat motor neurons (FIG. 8B) HESC-derived motor neurons in a co-culture system or (FIG. 8C) direct differentiation, without astrocyte feeder layer. (FIG. 8D) RT-PCR for the endogenous human Hb9 transcript on Hb9::GFP-positive (+) or -negative (−) fluorescent sorted cells. Scale bars: A, 80 μm and B-C, 40 μm.

FIGS. 9A-C show HESC-derived neuronal co-cultures with human fibroblasts. (FIG. 9A) Experimental design: Human primary fibroblasts were infected with LentiSOD1^{WT} or LentiSOD1^{G37R} for SOD1 overexpression. HESCs were differentiated into motor neuron precursors and plated on the two different fibroblasts conditions. (FIGS. 9B-C) Motor neurons were detected and quantified by Hb9 and TuJ1 immunofluorescence. Scale bar: 80 μm.

FIGS. 10A-G show Nurr1 suppression of LPS-induced inflammation and loss of TH+ neurons. (FIG. 10A) TH-DAB staining of a representative brain section of mice injected with shCtrl- or shNurr1-lentivirus and LPS is shown at AP −3.3 mm (Franklin and Paxinos, 2007).

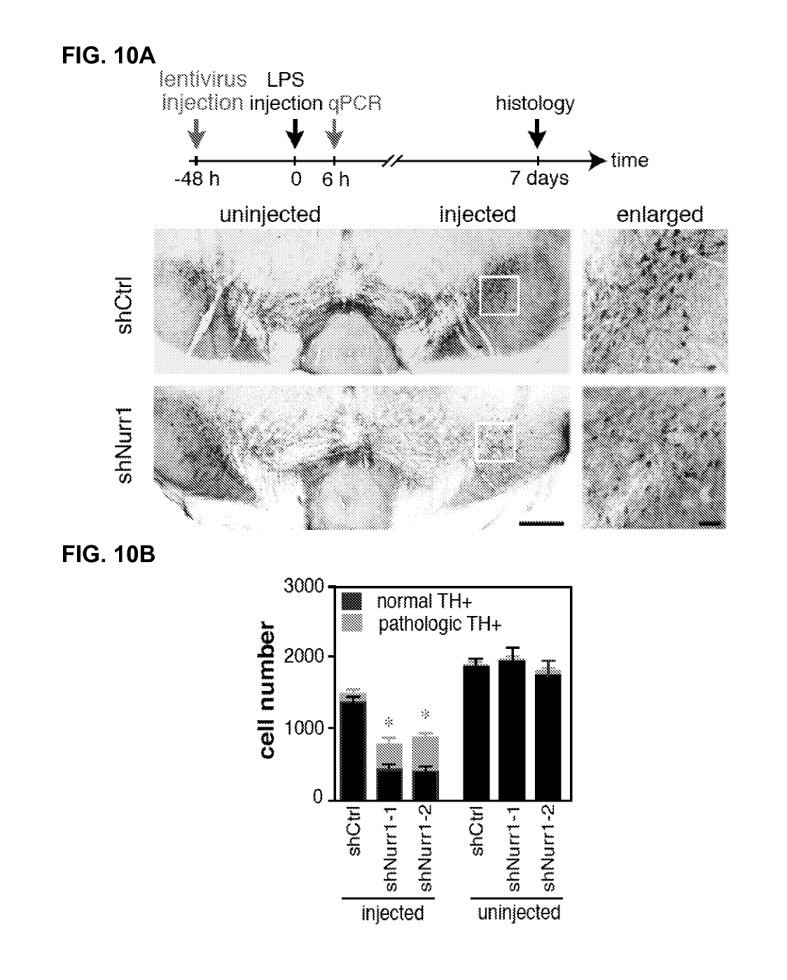

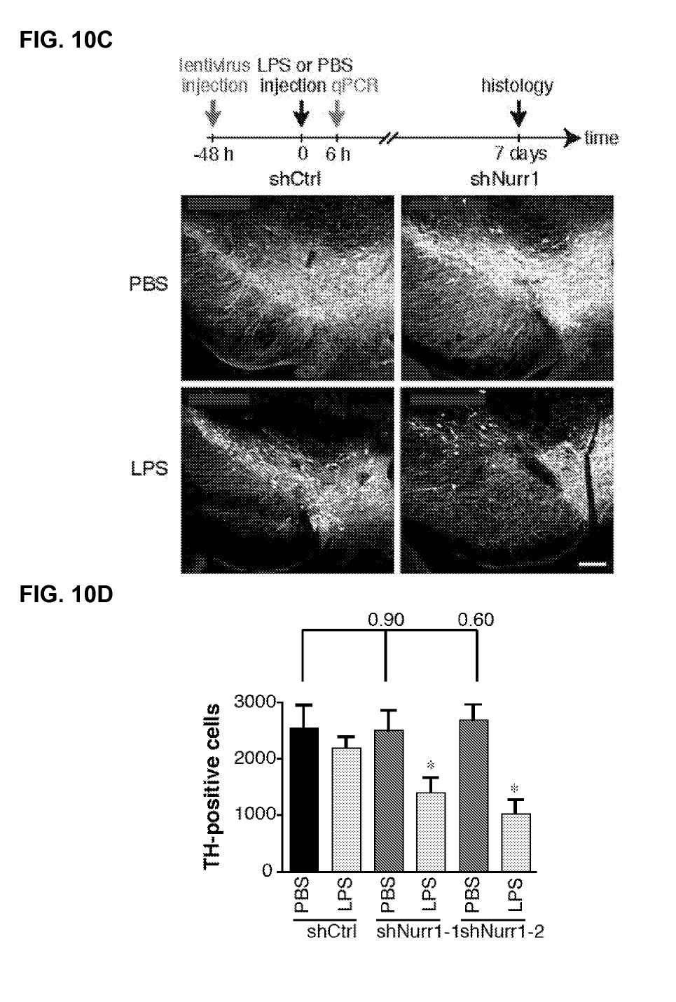

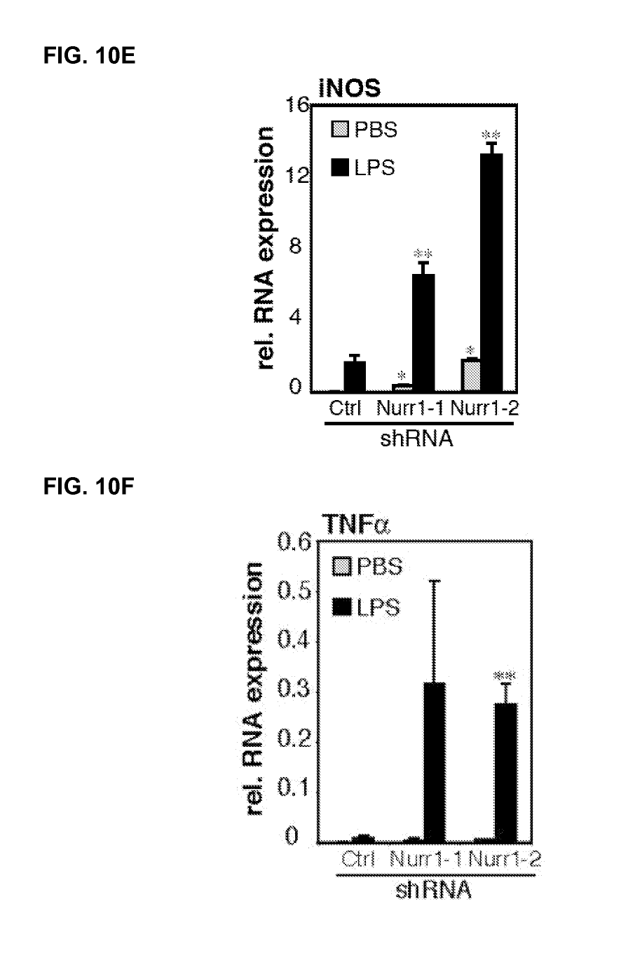

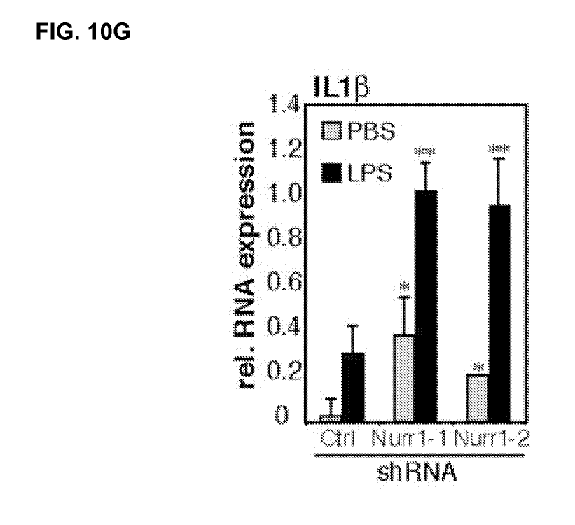

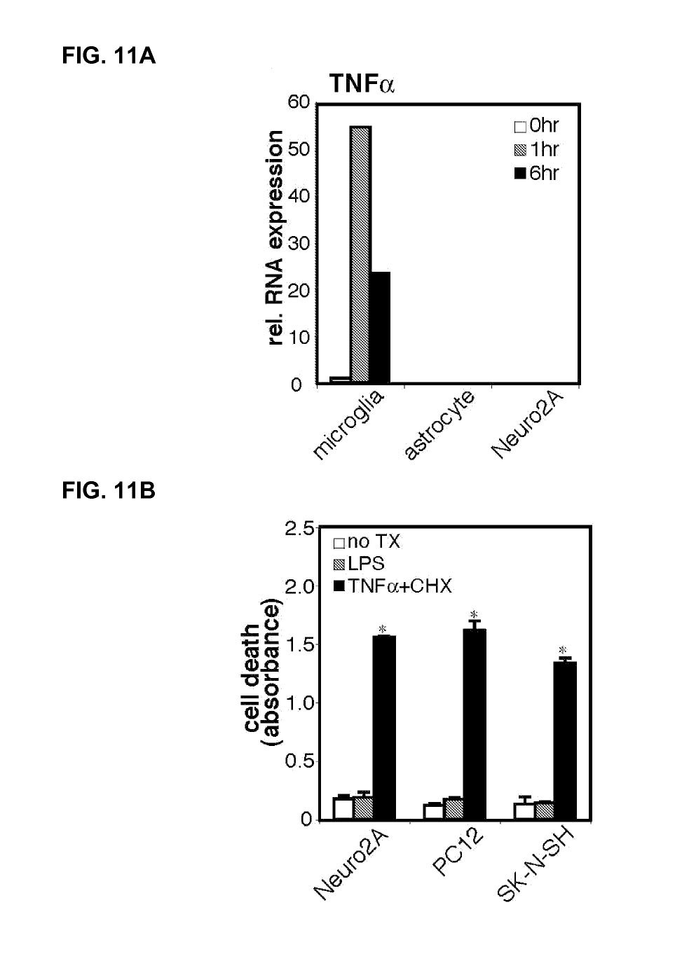

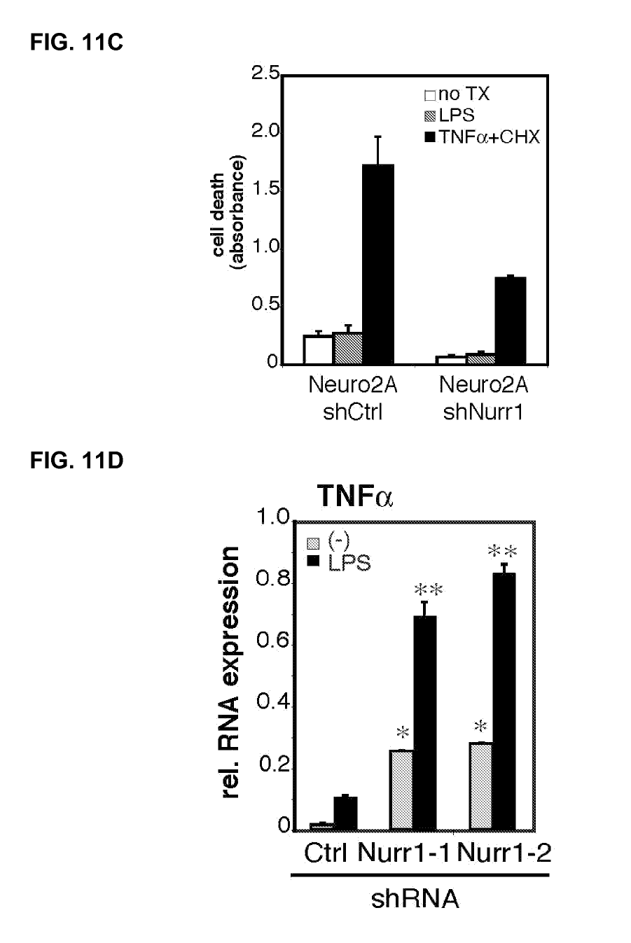

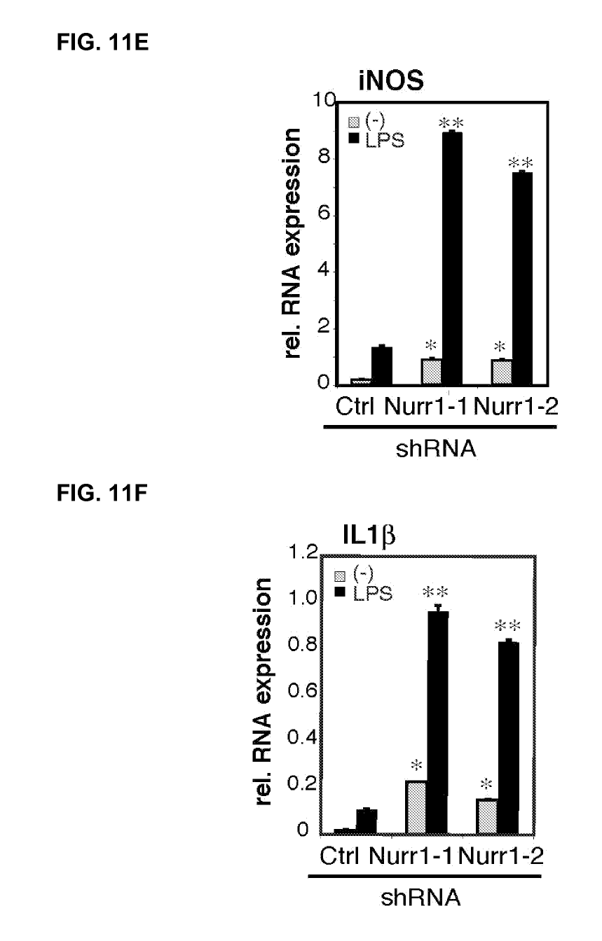

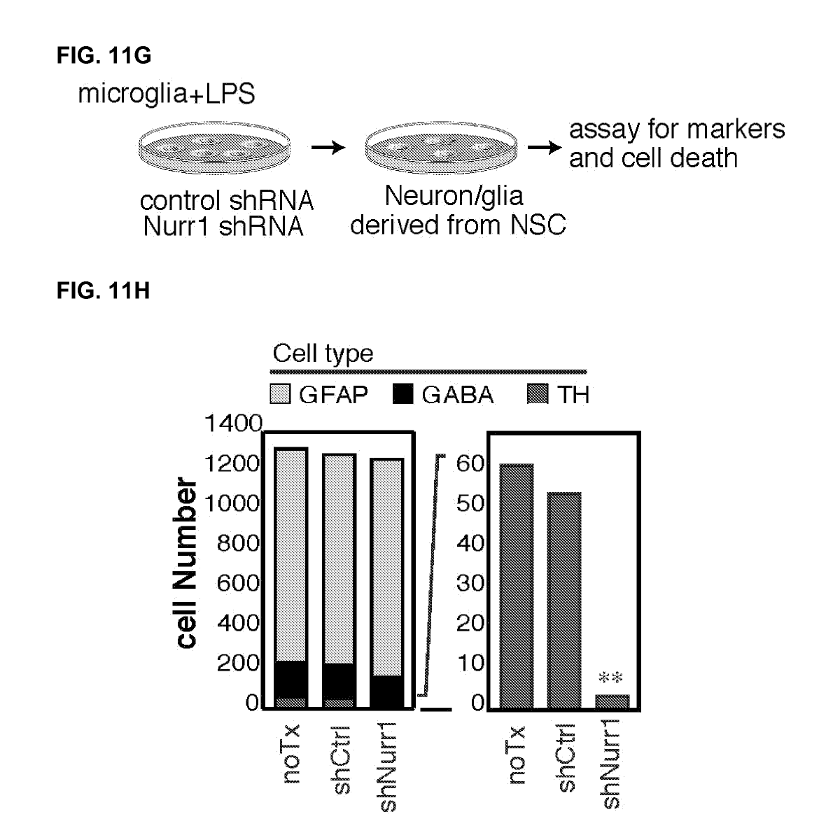

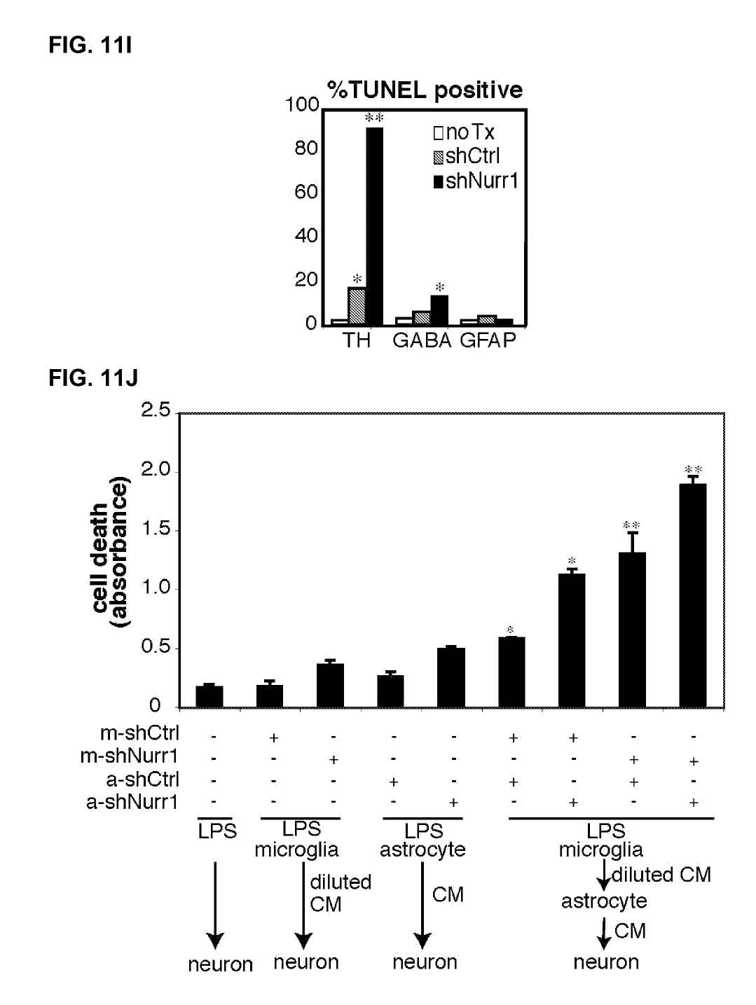

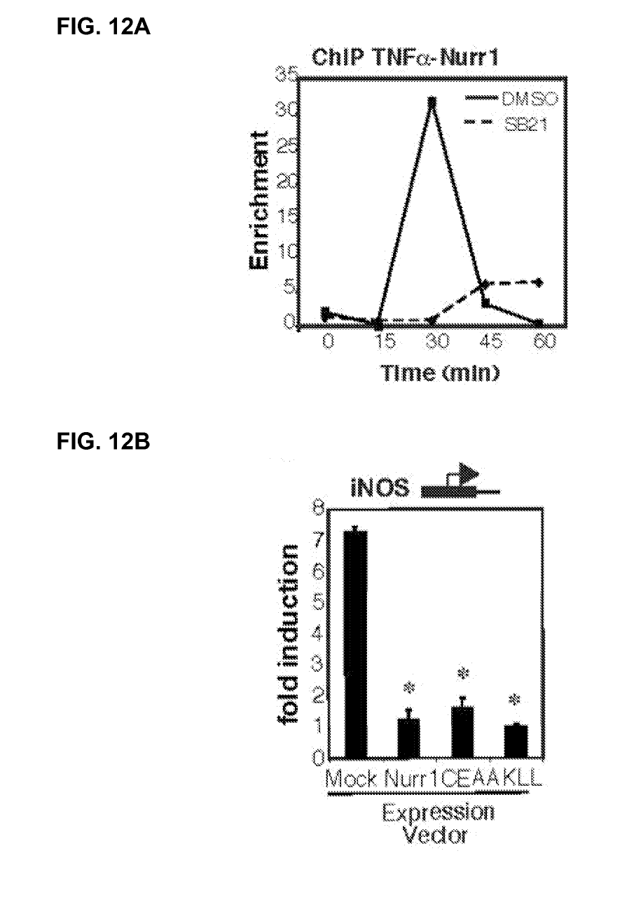

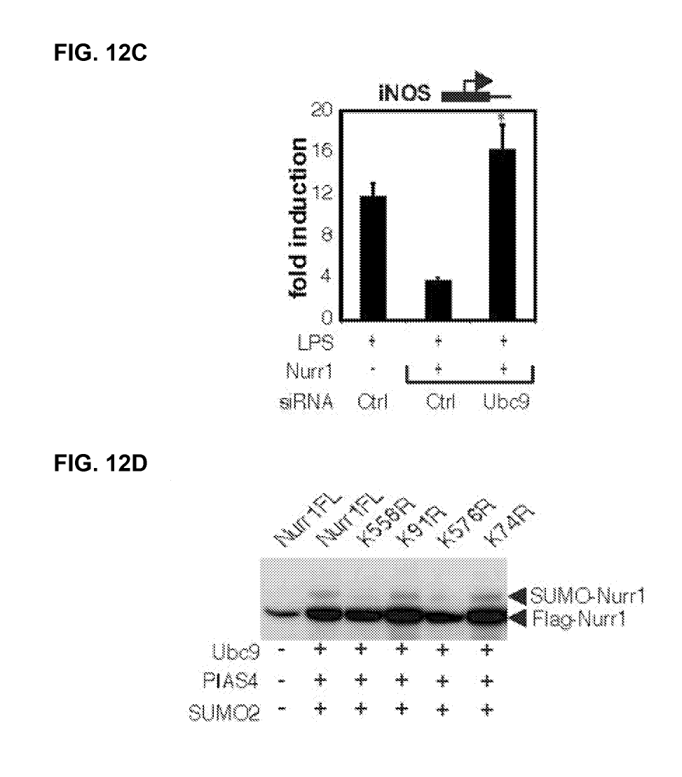

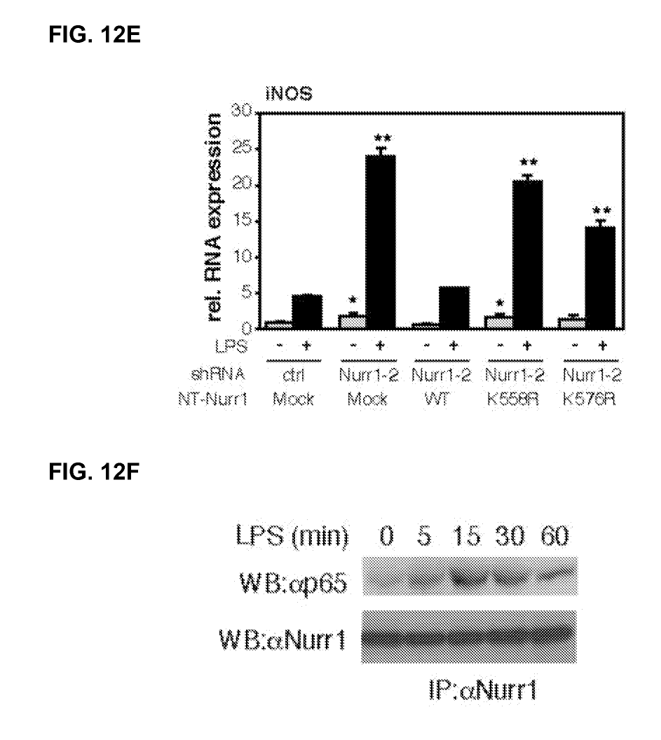

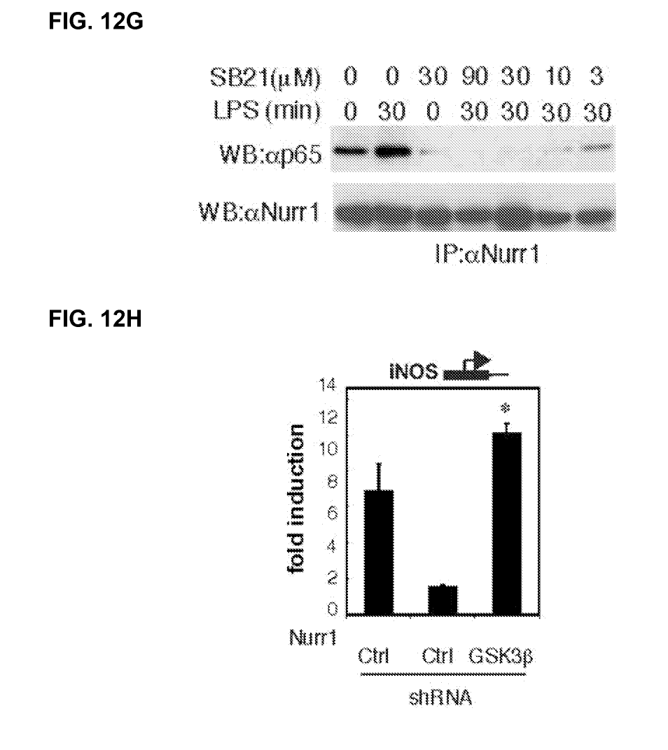

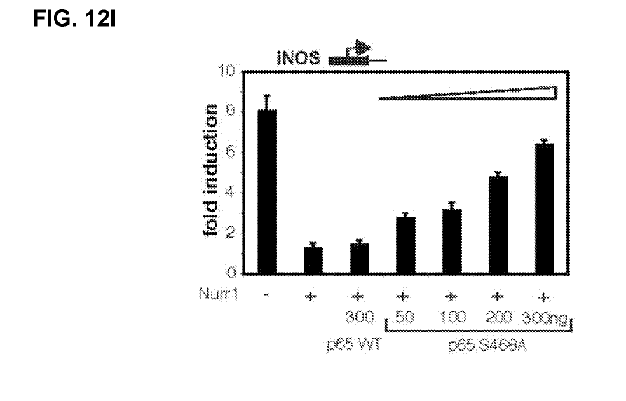

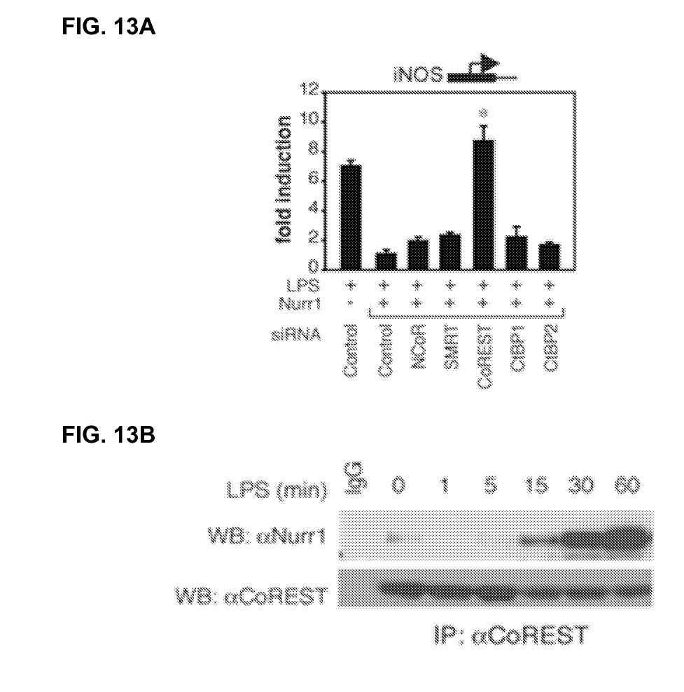

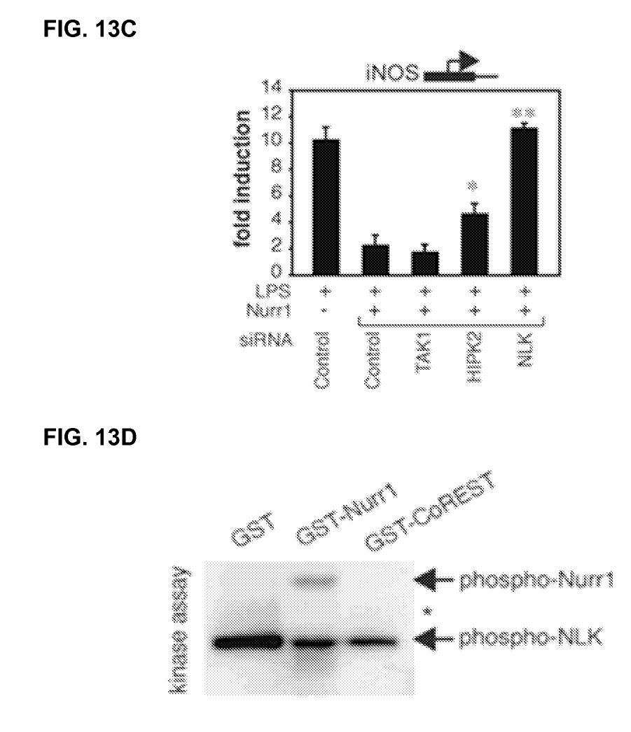

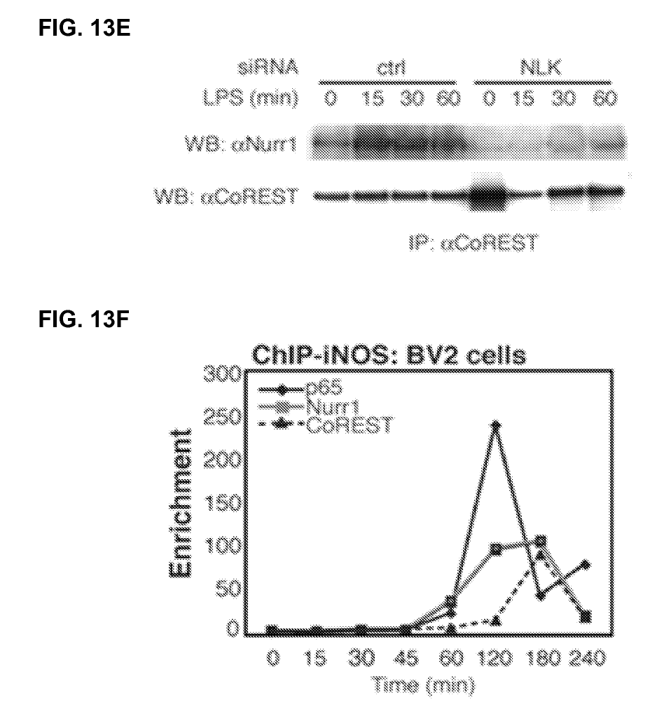

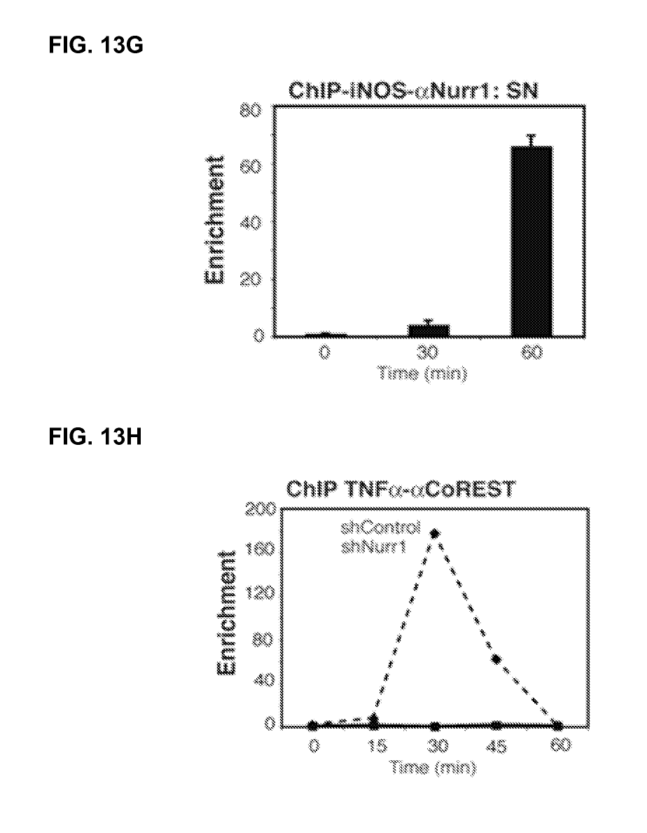

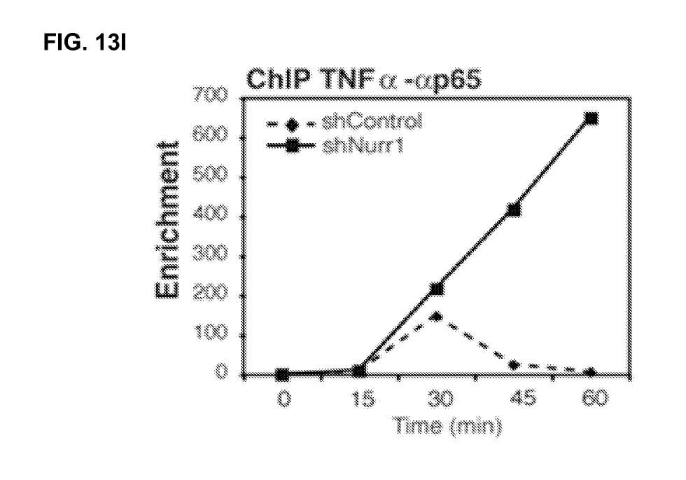

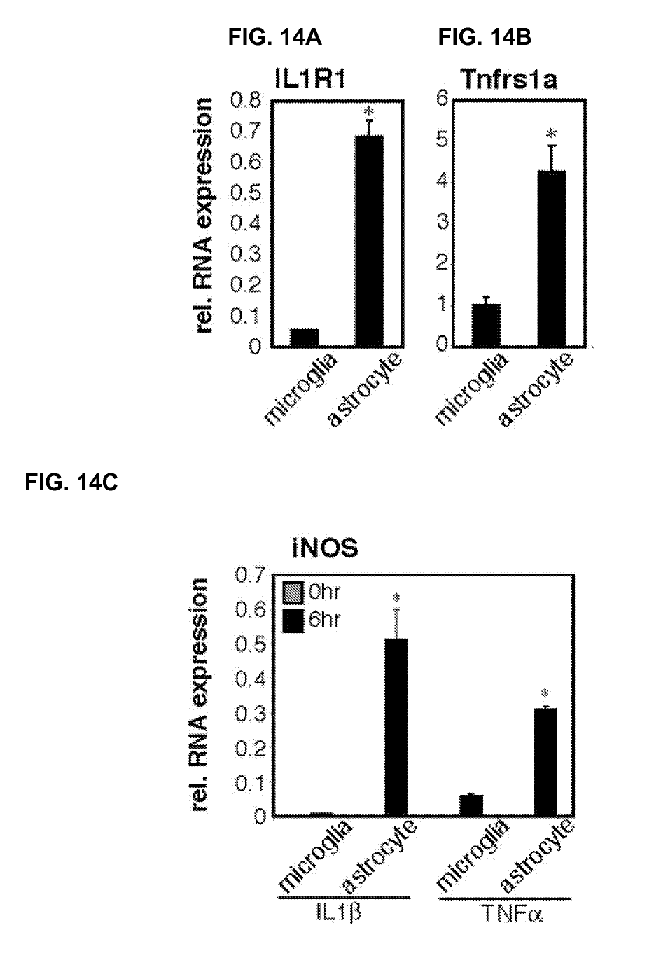

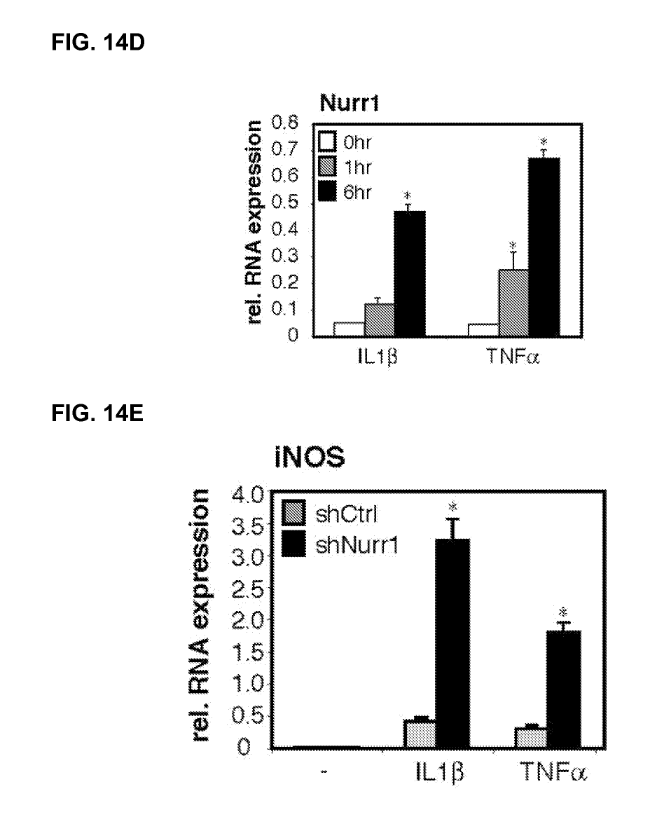

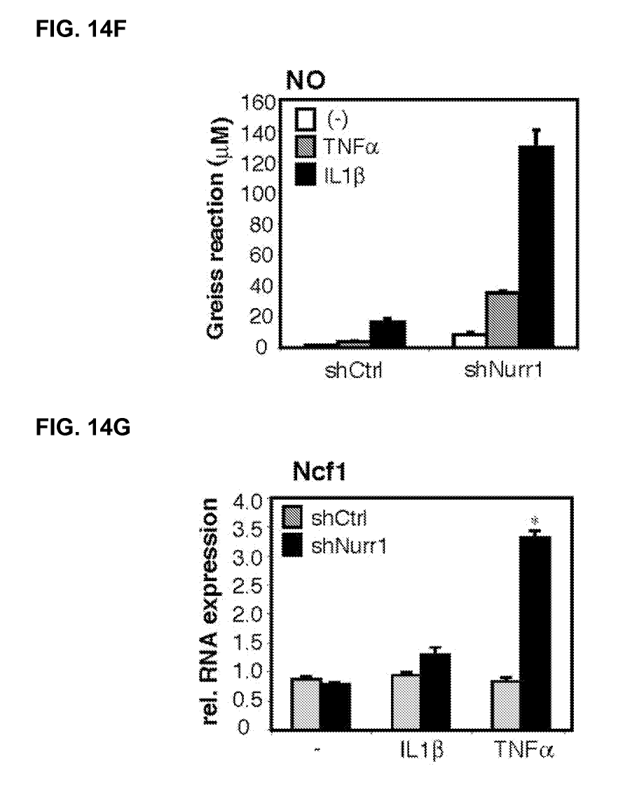

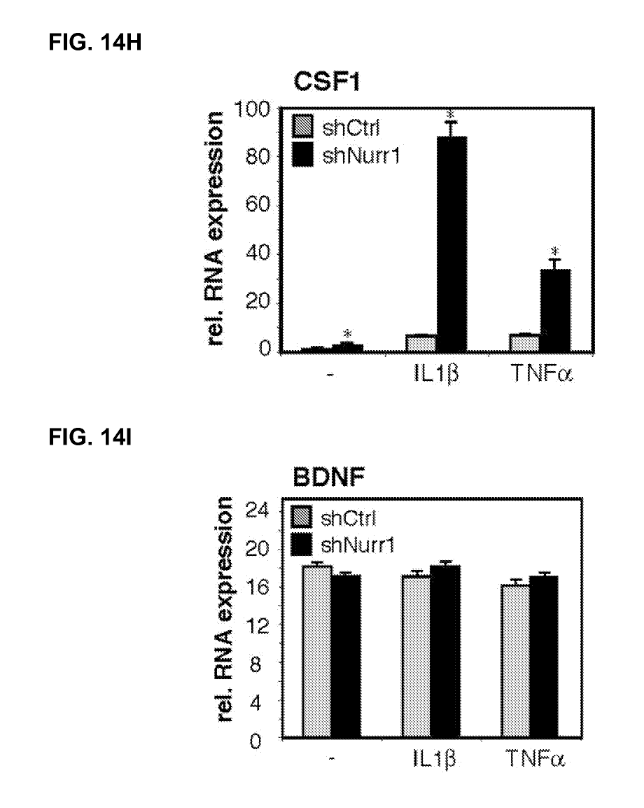

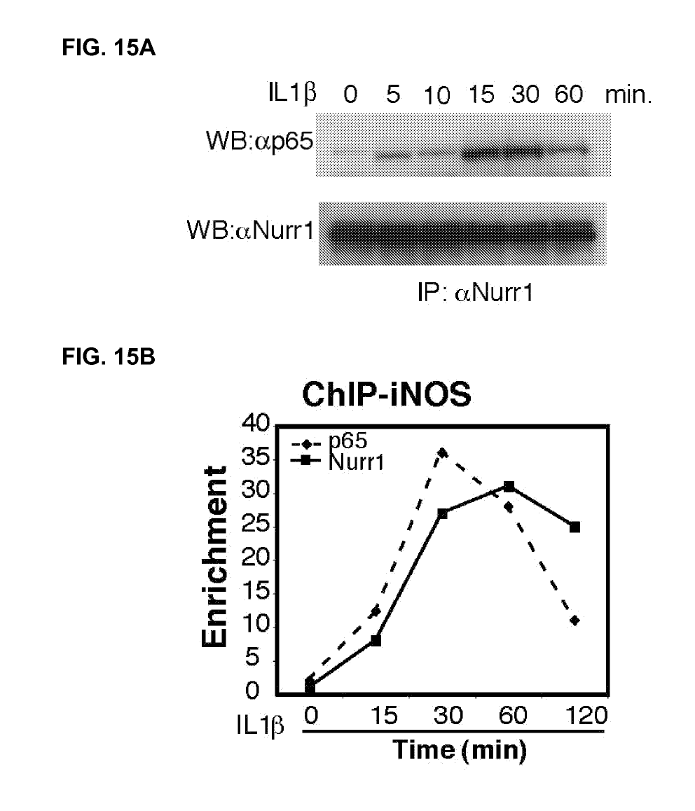

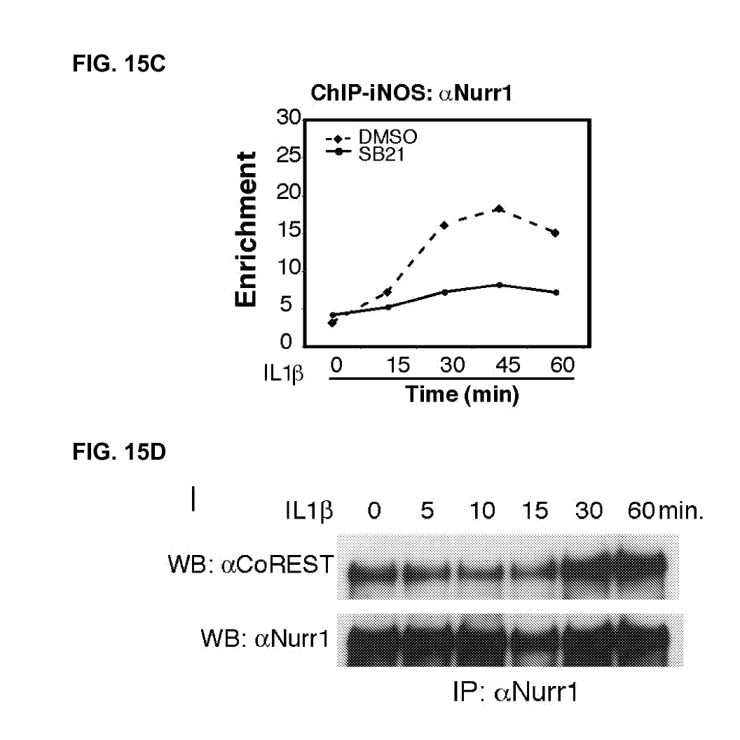

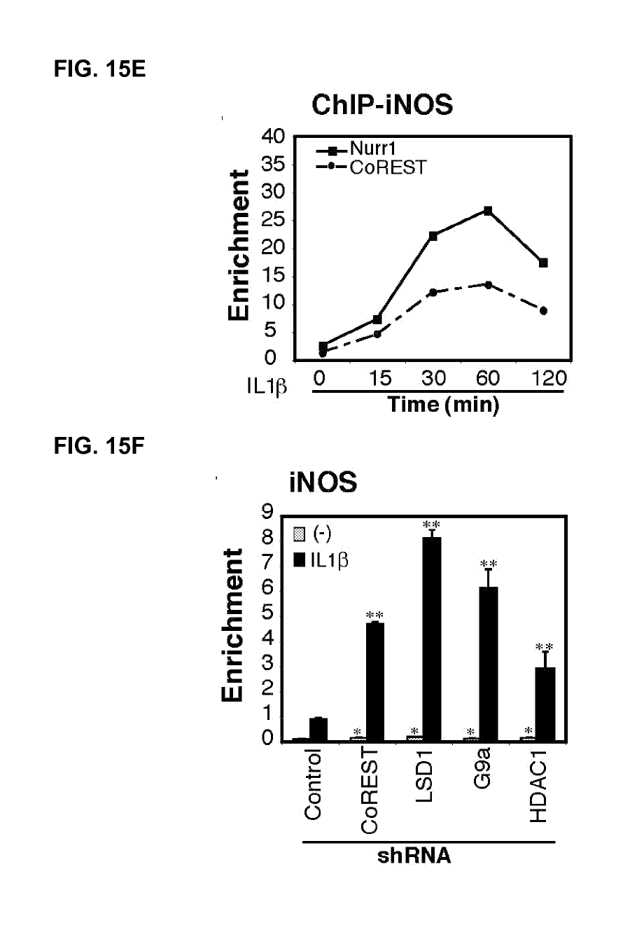

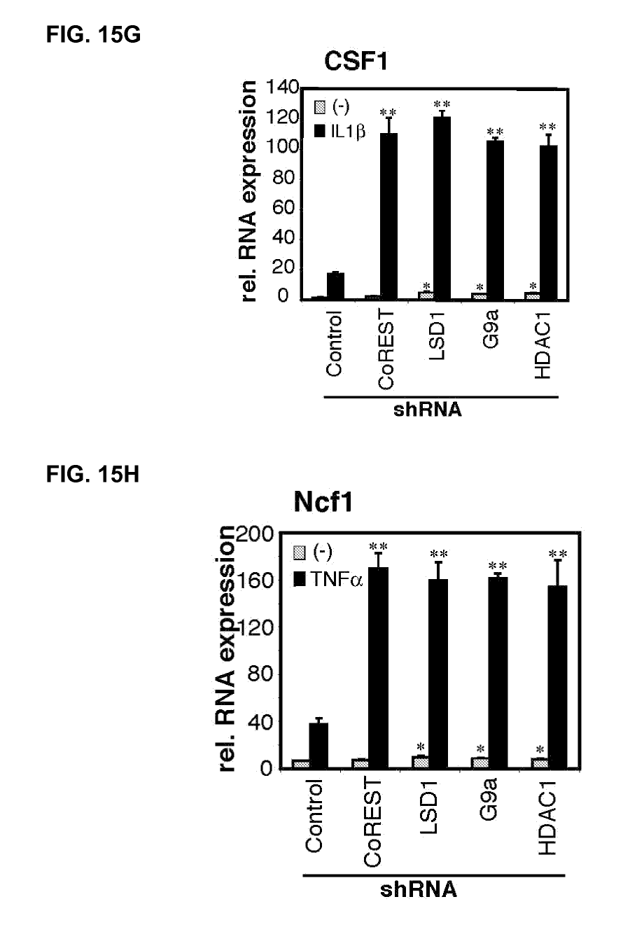

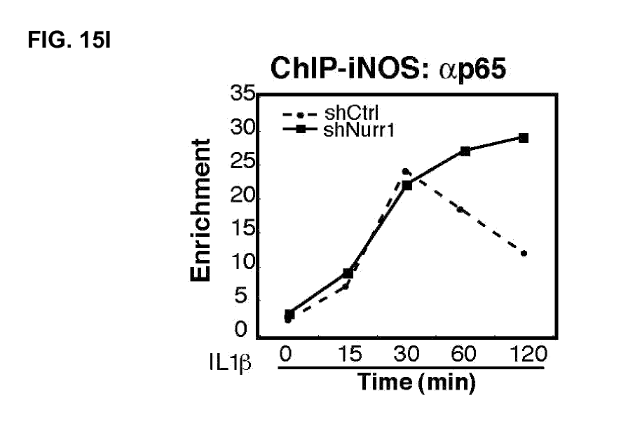

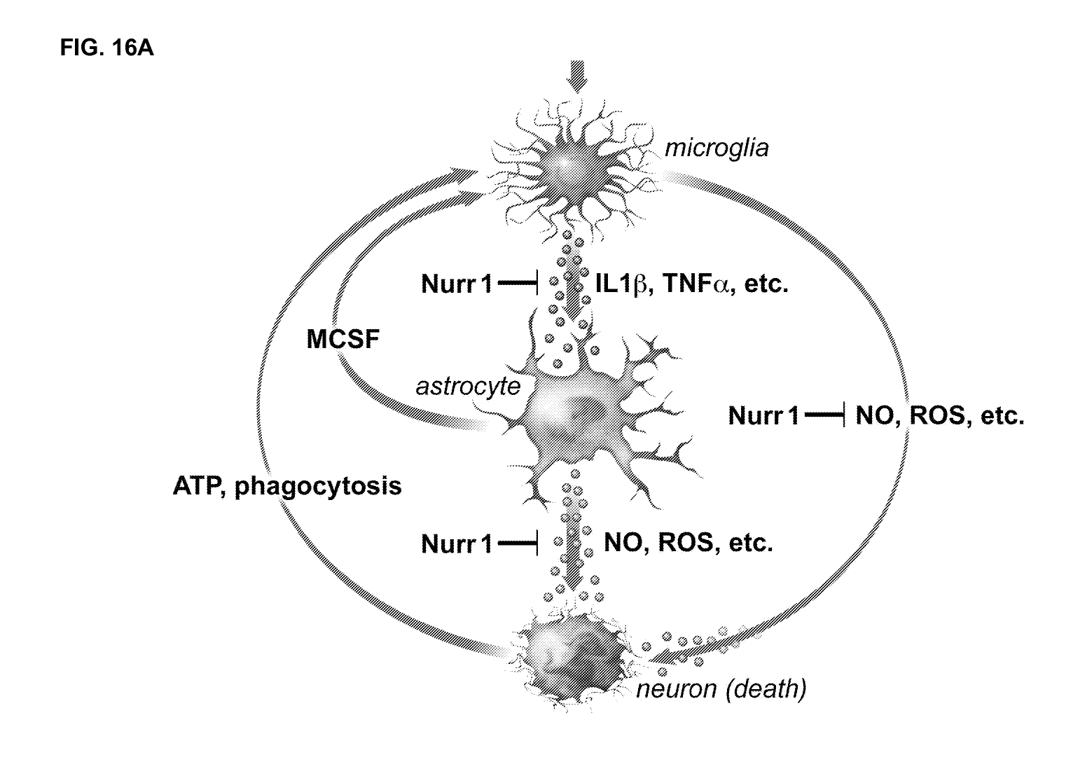

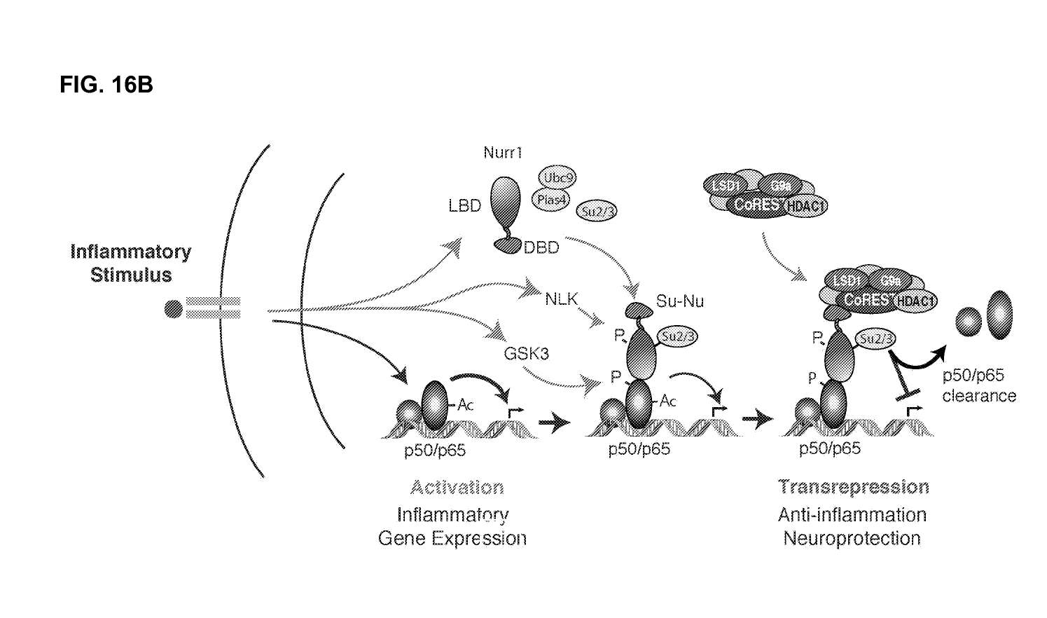

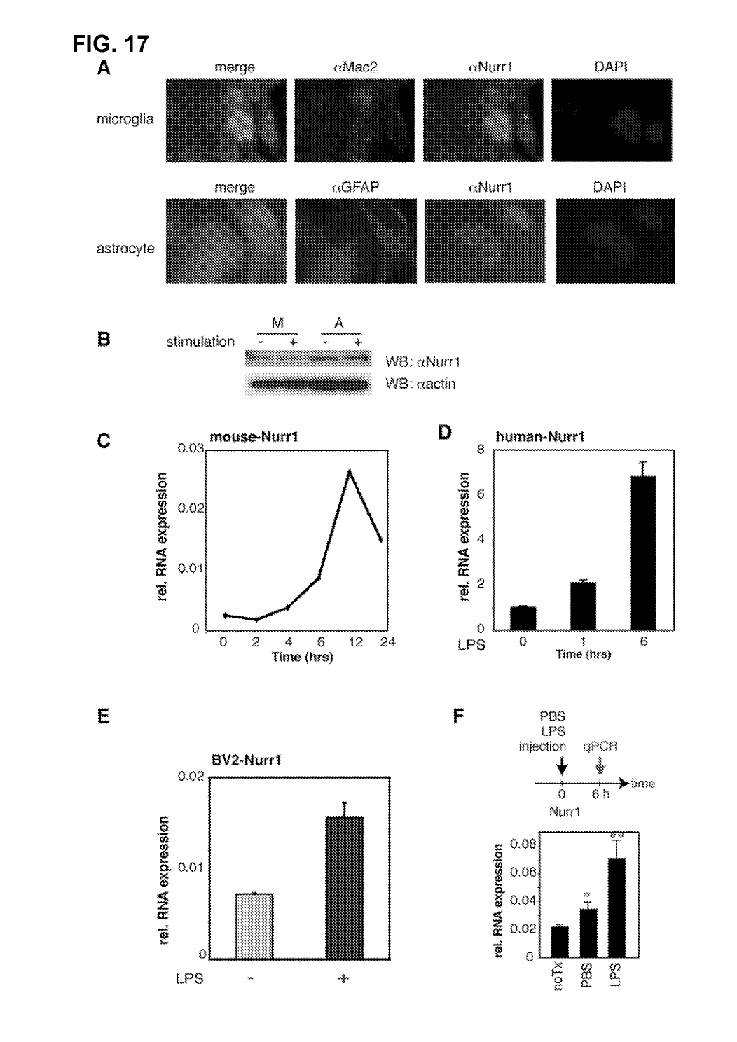

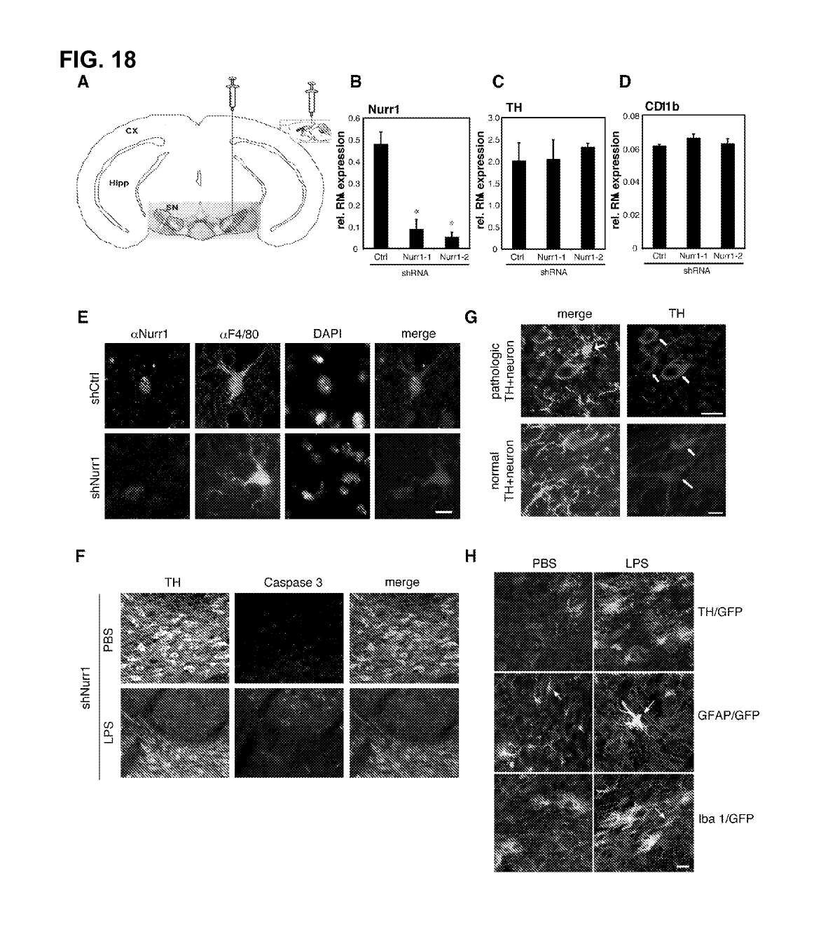

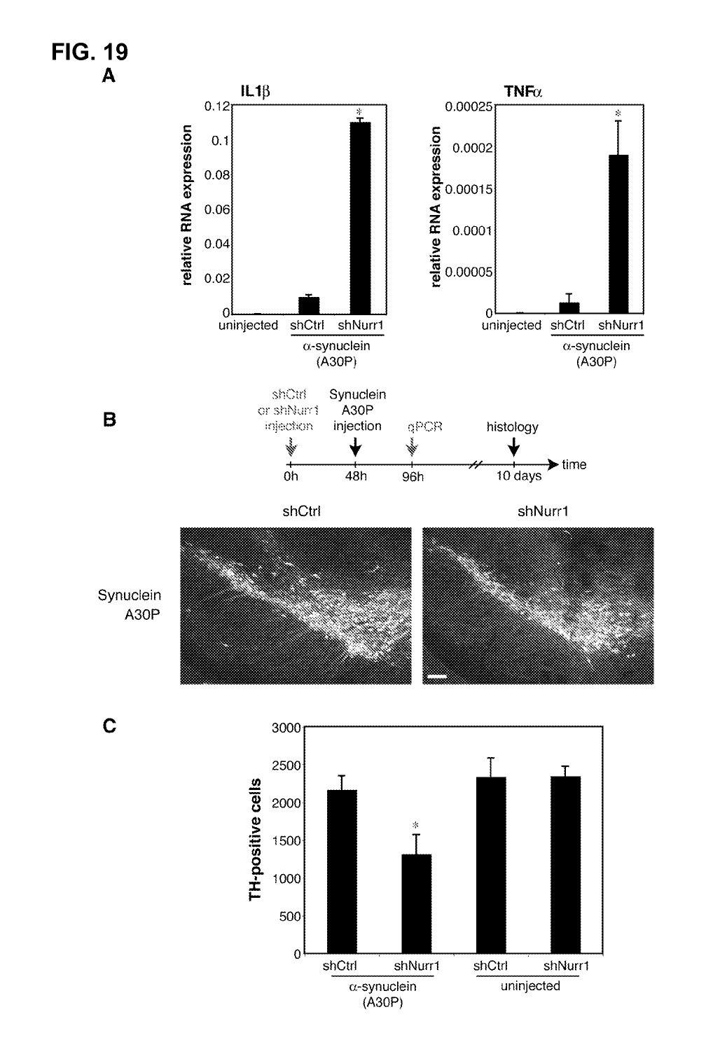

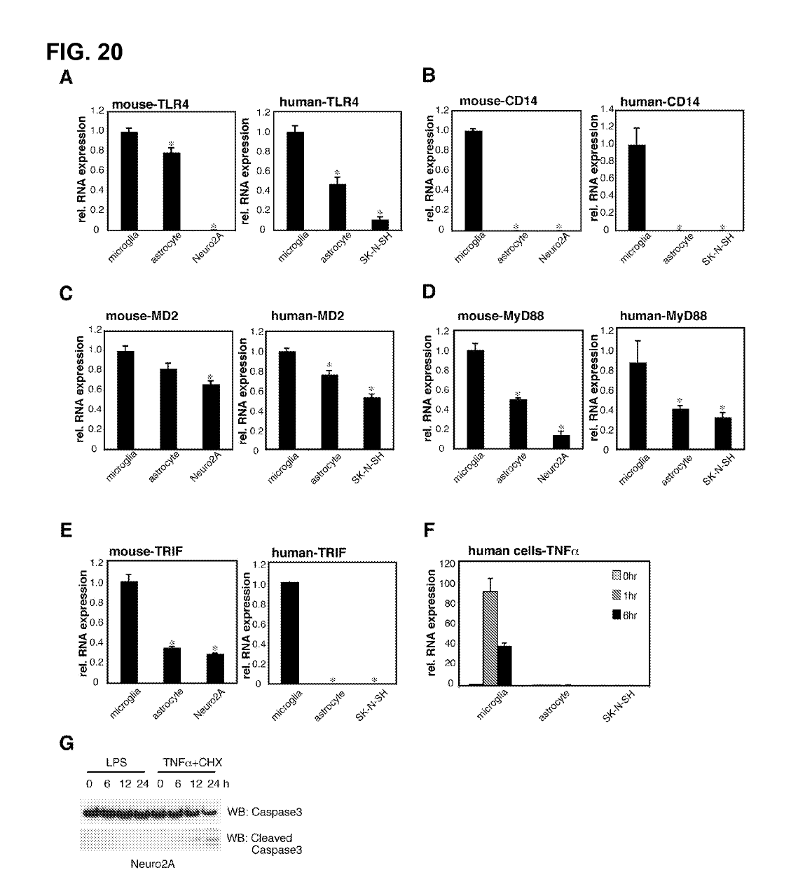

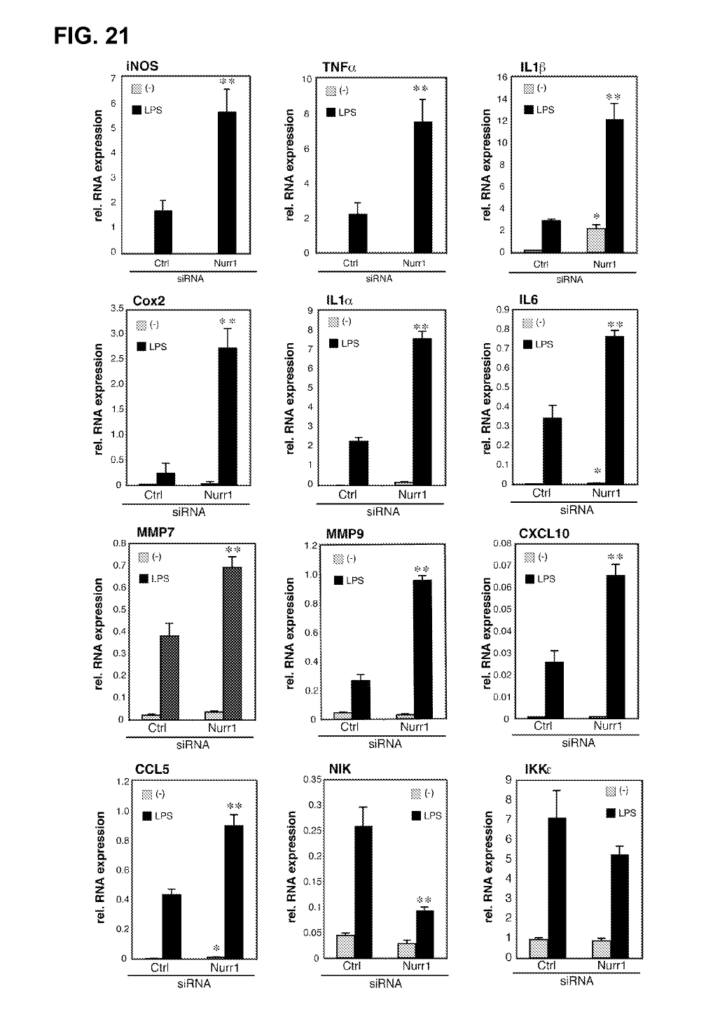

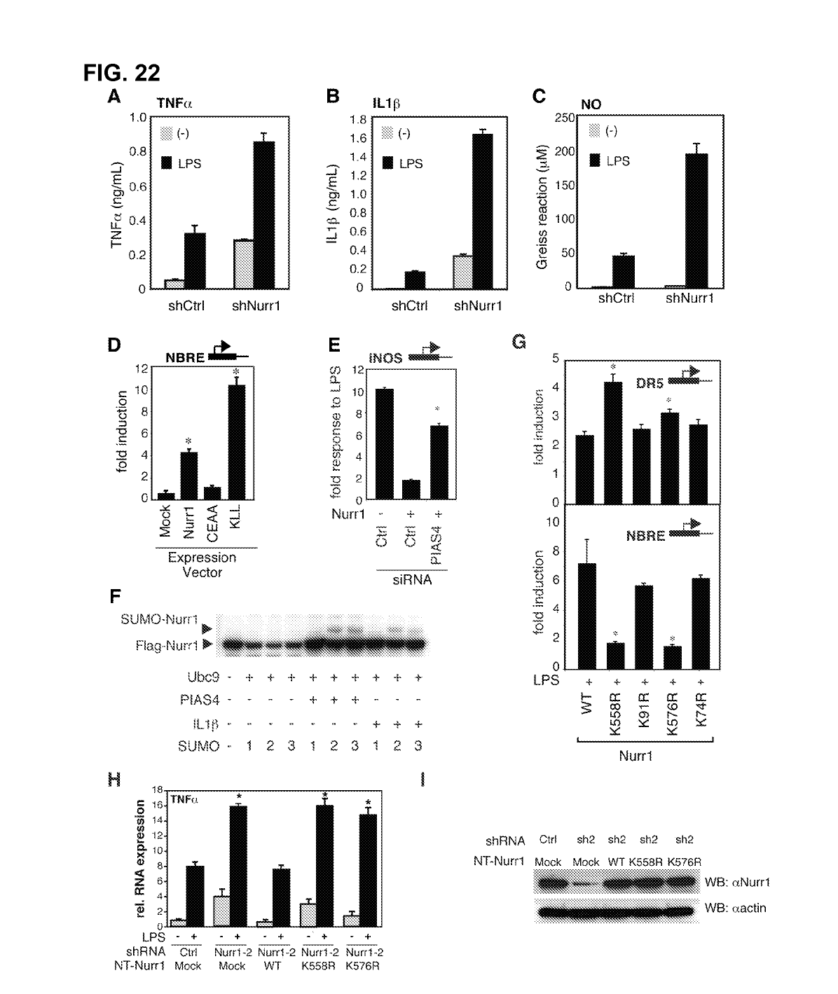

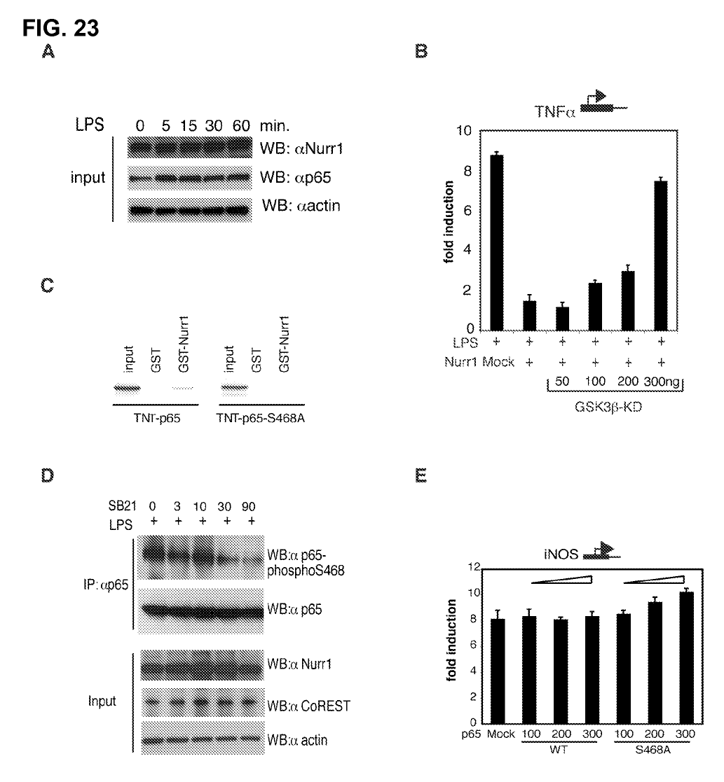

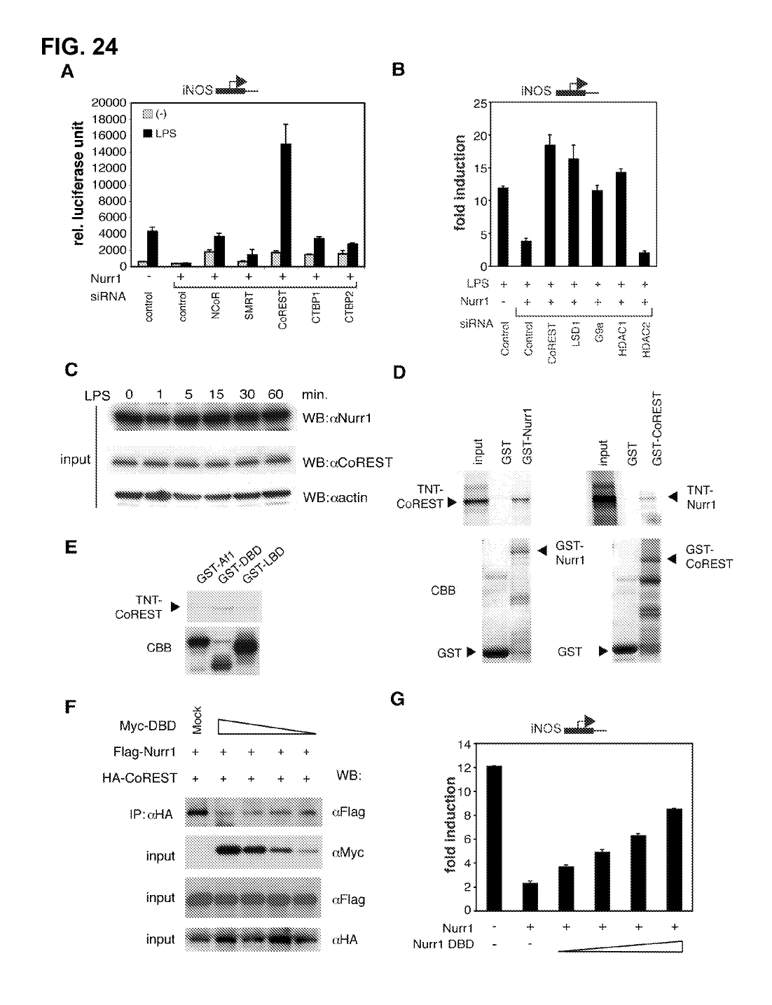

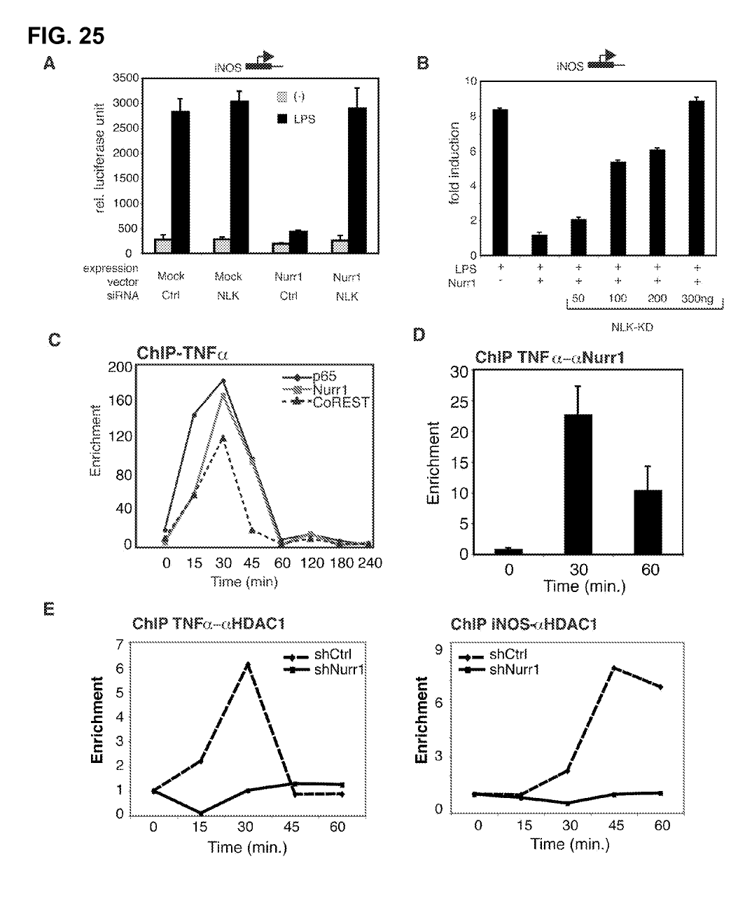

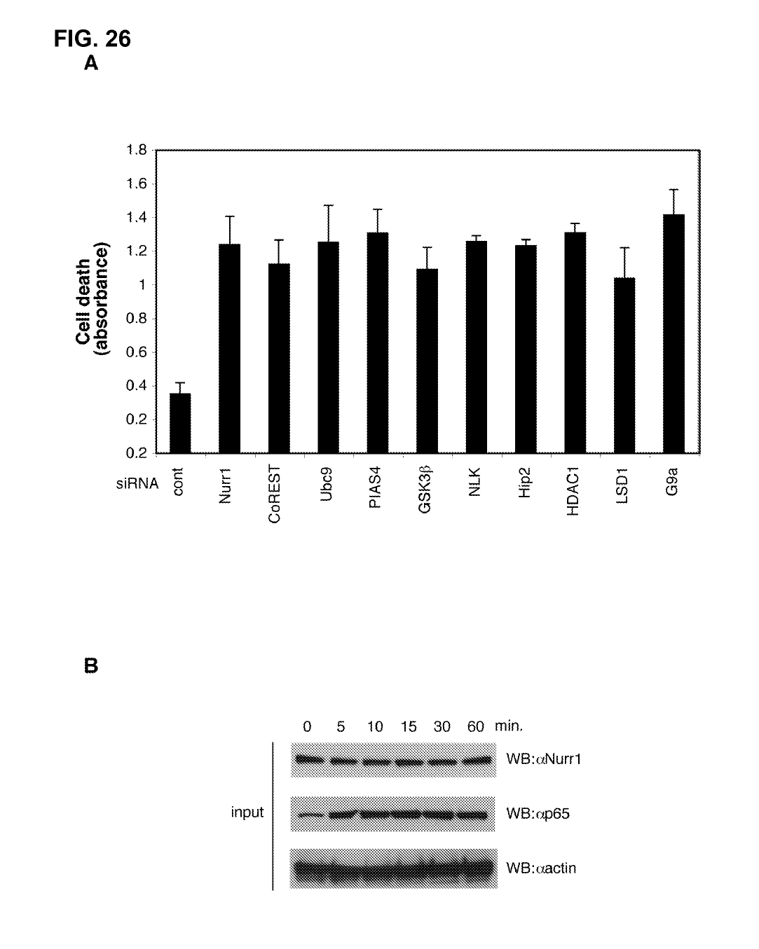

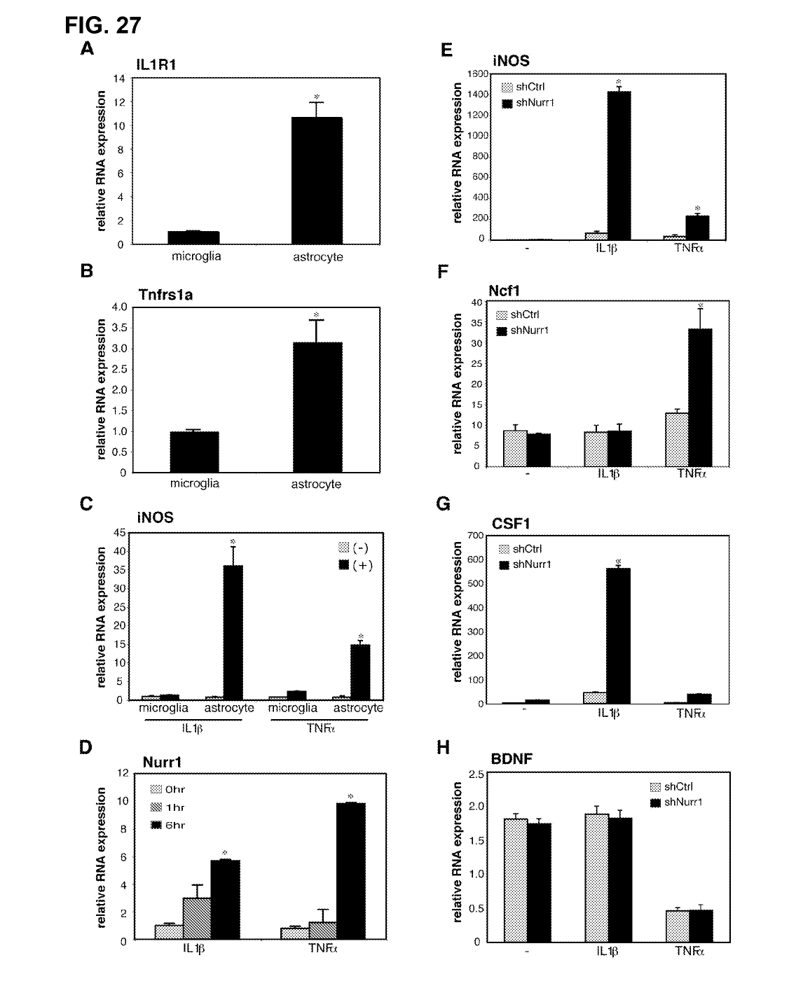

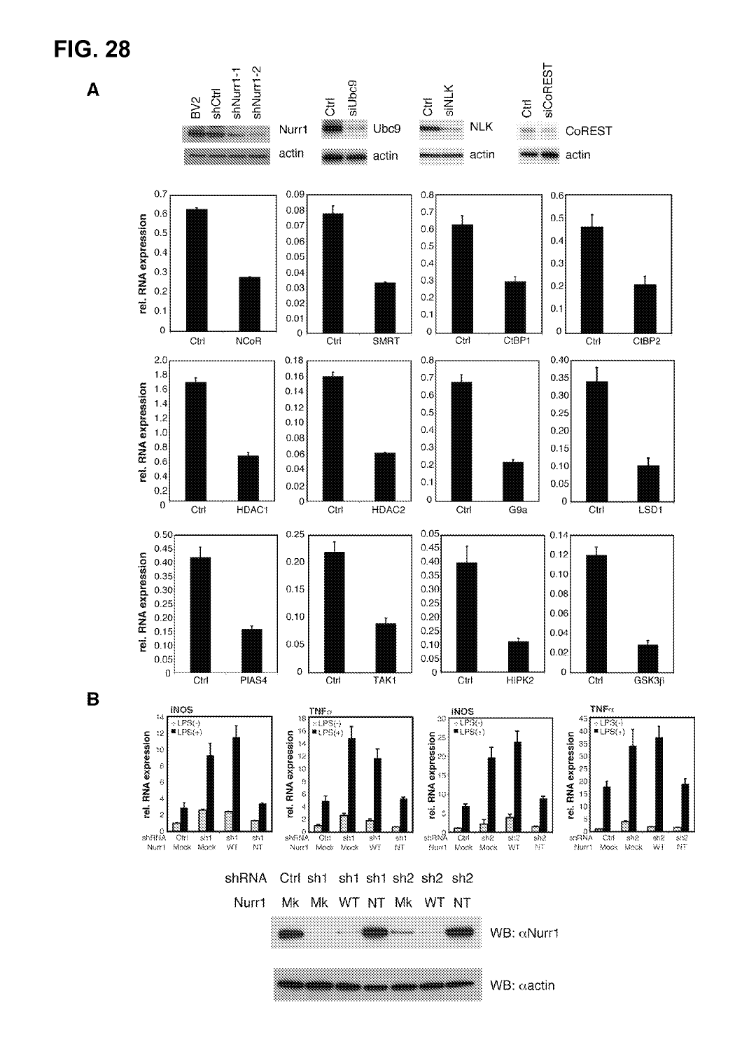

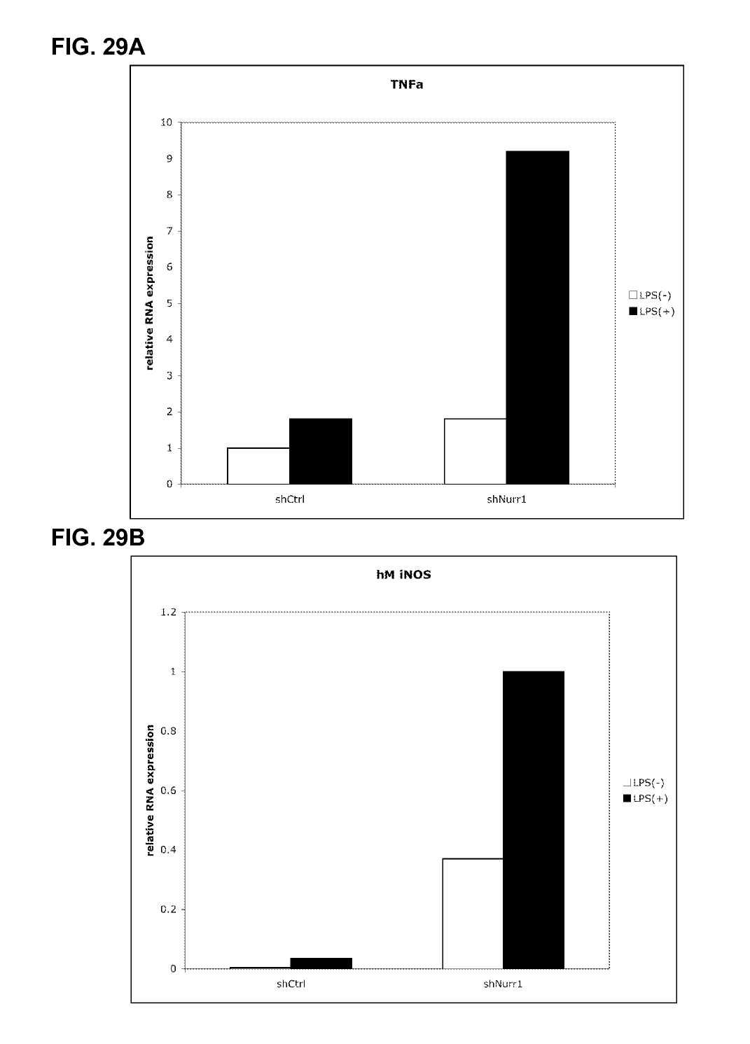

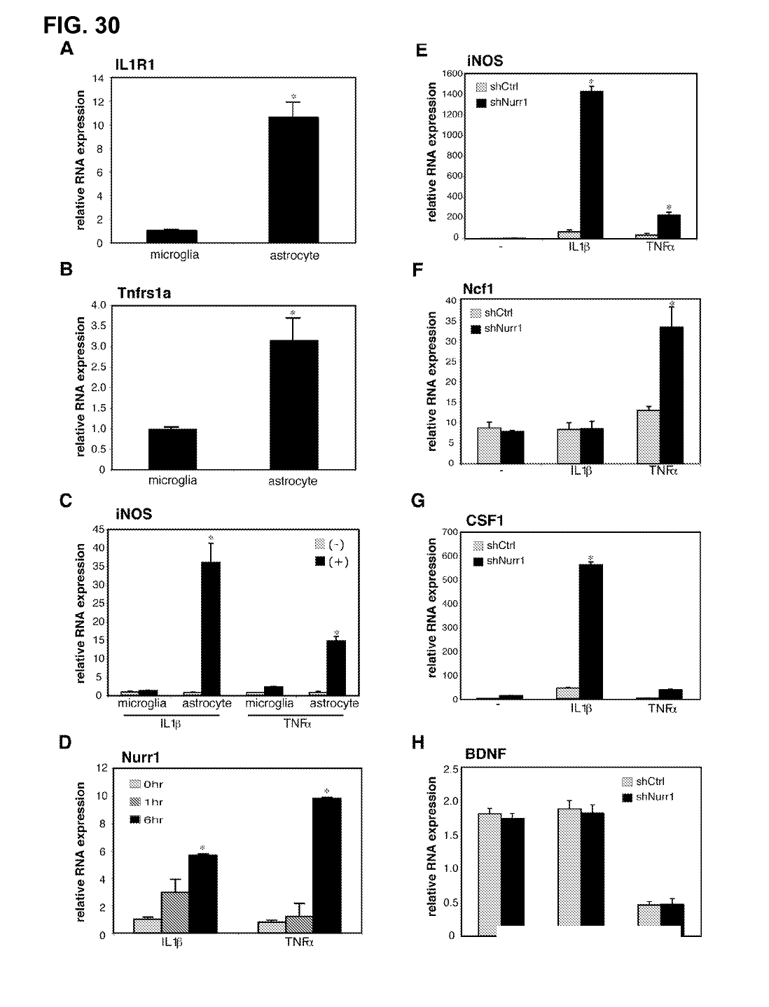

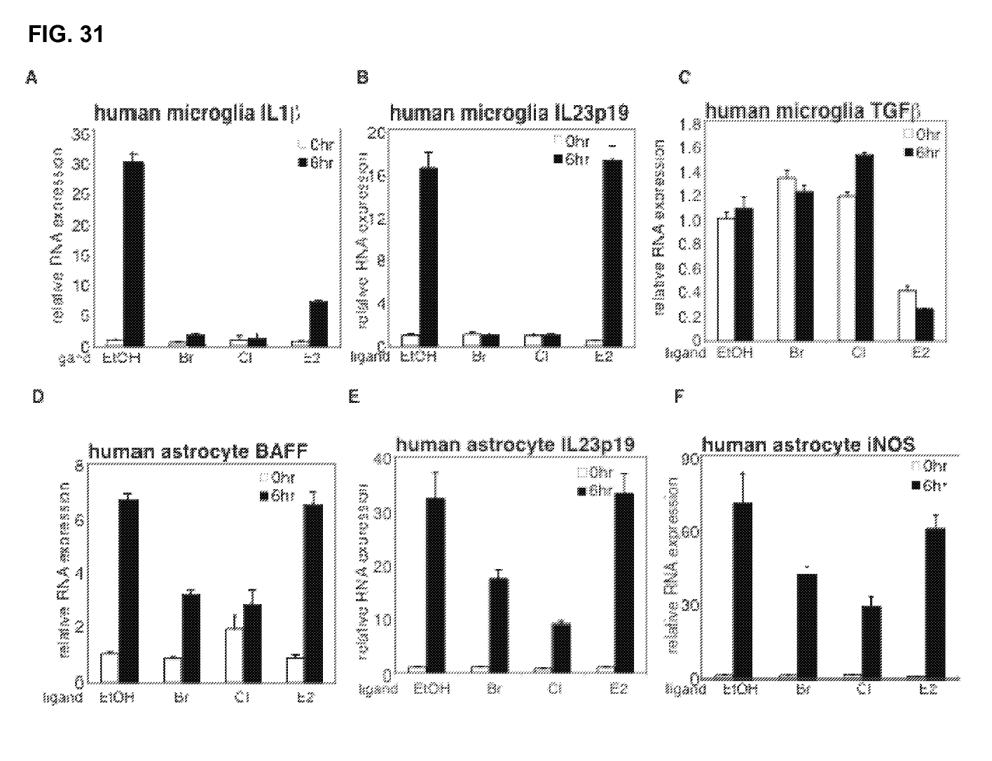

The left panels show an overview over the SN on the injected and uninjected side in shCtrl-(upper left panel) and shNurr1- (lower left panel) injected mice. Regions indicated by a rectangle in the injected side of the brain are enlarged in the right panels. Scale bars: 200 μm, right panels and 50 μm, left panels. (FIG. 10B) Stereological results of TH+ cell numbers indicate a significant decrease in TH+ neurons in the shNurr1 groups compared to the shCtrl-injected and the uninjected side. Discrimination between numbers of normal (black)/pathological (gray) TH+ neurons indicates that this decrease is accompanied by a larger fraction of pathological TH+ cells in the shNurr1 groups. See Examples for explanation of normal/pathological TH+ neurons. Asterisk, p<0.01 compared to the numbers from shCtrl-lentivirus-injected and the uninjected side. (n=5) (FIG. 10C) Fluorescence-TH staining of a representative brain section of mice injected with shCtrl- or shNurr1-lentivirus followed by PBS or LPS, as described in A. Experimental diagram is indicated at the top. Scale bar 200 μm. (FIG. 10D) Stereological results of TH+ cell numbers indicate a significant decrease in TH+ neurons only in the setting of Nurr1 knockdown followed by LPS injection. Asterisk, p<0.002 compared to PBS injection (n=4). Knockdown of Nurr1 alone did not affect TH+ cell numbers at the 7-day time point (p=0.90 shNurr1-1/PBS-injected group and p=0.60 shurr1-2/PBS-injected group compared to shCtrl/PBS group). (FIGS. 10E-G) Expression of iNOS (FIG. 10E), TNFα (FIG. 10F) and IL1β (FIG. 10G) mRNA in Nurr1-knockdown SN 6 hours after LPS injection as determined by qPCR and normalized to HPRT expression (n=4). Error bars represent SD. Asterisk, p<0.01 compared to shCtrl/PBS-injected; **, p<0.01 compared to shCtrl/LPS-injected samples.

FIGS. 11A-J show microglia initiated LPS-mediated inflammation and astrocytes propagating the production of neurotoxic factors. (FIG. 11A) Primary microglia increased TNFα mRNA upon LPS stimulation but not primary astrocytes or Neuro2A cells. Cells were stimulated with LPS for the indicated time and mRNA was quantified by qPCR as described before. (FIG. 11B) Neuronal cell lines were treated with 0.1 μg/ml LPS (gray) or 50 ng/ml TNFα plus 10 μg/ml Cycloheximide (CHX) (black) for 24 h, and effects on viability were determined using a TUNEL ELISA assay. *, p<0.01 compared to untreated sample (no Tx) (white). (FIG. 11C) Knockdown of Nurr1 in Neuro2A cell did not increase sensitivity to TNFα+CHX. Neuro2A cells were infected with shCtrl- or shNurr1-lentivirus and treated with LPS (gray) or TNFα plus CHX (black) as described in FIG. 11B. Viability of the cells was determined using a TUNEL ELISA assay. (FIGS. 11D-E) shNurr1-BV2 cells expressed higher levels of TNFα (FIG. 11D), iNOS (FIG. 11E) and IL1β (FIG. 11F) mRNA. shCtrl or shNurr1-BV2 cells were stimulated with 0.1 μg/ml LPS and mRNA levels were determined by qPCR. *, p<0.01 compared to no stimulation (Ctrl); **, p<0.01 compared to LPS stimulation of shCtrl-BV2 cells. G. Scheme of conditioned media (CM) and cell death assay. CMs were harvested from shCtrl- and shNurr1-BV2 cells that were stimulated with LPS for 24 h (0.1 μg/ml). Neurons or glial cells were assayed for specific markers by immunostaining and for cell death by TUNEL assay. (FIG. 11H) CM from LPS-treated shCtrl-BV2 cells and a mixture of the CMs from shNurr1-1- and shNurr1-2-infected BV2 cells (shNurr1) were incubated with neurons and glial cells derived from in vitro differentiated neural stem cells (NSC). TUNEL assay was performed on TH-, GABA- or GFAP-positive cells derived from mouse NSC. Numbers of TUNEL-negative live cells are shown. TH-positive cells are indicated. (FIG. 11I) The percentages of TUNEL-positive population are shown. *, p<0.01 and **, p<0.001 compared to no treatment (no TX). (FIG. 11J) Knockdown of Nurr1 in microglia increases and astrocytes enhance the production of neurotoxic factors. Primary mouse microglia and astrocytes were infected with shCtrl- or shNurr1-lentivirus. Cells were treated with 0.1 μg/ml LPS for 2 h and washed extensively with PBS. Cells were kept cultured for another 24 h with fresh medium. For sequential CM assay, CMs harvested from microglia were cultured with lentivirus-infected astrocytes for 24 hours. Then, CMs were harvested and the viability of Neuro2A cells was measured as described before.

FIGS. 12A-I show Nurr1 acting as an RXR-independent, GSK3β-dependent transrepressor for NF-κB. (FIG. 12A) Nurr1 is recruited to the TNFα-promoter in response to LPS. This recruitment is blocked by the GSK3β-specific inhibitor SB216763 (SB21). BV2 cells were pre-incubated with DMSO or 30 μM SB21 for 1 h followed by LPS stimulation for the indicated times before ChIP assay. Data are displayed as fold enrichment over control IgG. (FIG. 12B) Nurr1-mediated repression is independent of DNA binding and RXR dimerization. RAW264.7 cells were transfected with wild type Nurr1, P-box mutant Nurr1C280A/E281A (CEAA) and I-box mutant Nurr1K555A/L556A/L227A (KLL). iNOS-promoter activity in RAW264.7 cells measured by luciferase-reporter assay. *, p<0.01 compared to control (Mock). (FIG. 12C) siRNA against Ubc9 abolishes Nurr1-mediated transrepression of iNOS-promoter activity. *, p<0.01 compared to Nurr1 with control siRNA. (FIG. 12D) K558 and K576 are the predominant SUMOylation sites of Nurr1. Flag-tag SUMOylation mutants of Nurr1 were transfected into Hela cells. SUMO assay was performed as described in Examples. (FIG. 12E) Nurr1-mediated repression of iNOS-promoter activity is dependent on SUMOylation of Nurr1 at K558 or K576. Flag-tagged wild-type Nurr1 (Nurr1-FL) or SUMOylation-site mutants were transfected into RAW264.7 cells and iNOS-luciferase assay was performed as described before. *, p<0.01 compared to Nurr1-FL transfection. (FIG. 12F) LPS stimulation enhances physical association of Nurr1 and p65 in BV2 cells. Lysates of BV2 cells stimulated with LPS (1 μg/ml) for the indicated times were immunoprecipitated with anti-Nurr1 antibody and Western blots were developed with anti-p65 antibody. Equal loading was determined by re-blotting using anti-Nurr1 antibody. (FIG. 12G) SB21 impairs the binding of Nurr1 and p65 in a dose-dependent manner. BV2 cells were incubated with SB21 at the indicated concentrations for 1 h prior to stimulation with LPS (1 μg/ml). IP and Western blotting were performed as described in FIG. 12F. (FIG. 12H) siRNA against GSK3β abolishes Nurr1-mediated repression of iNOS-promoter activity. Nurr1 expression vector was transfected into RAW264.7 cells together with control siRNA or siRNA against GSK3β and iNOS-promoter activity was determined as described before. *, p<0.01 compared to Nurr1 with control siRNA. (FIG. 12I) S468A mutant of p65 abolishes Nurr1-mediated repression of iNOS-promoter activity. iNOS-promoter activity was determined as described before.

FIGS. 13A-I show the CoREST repressor complex requirement for Nurr1-mediated repression. (FIG. 13A) Requirement for CoREST in Nurr1-mediated repression. iNOS-luciferase and Nurr1-expression or control vector as well as siRNAs against the indicated corepressors were transfected into RAW264.7 cells and iNOS-promoter activity was assayed as described in FIG. 12H. *, p<0.01 compared to Nurr1 with control siRNA. (FIG. 13B) Physical interaction of Nurr1 and CoREST in BV2 microglial cells. Co-IP was performed with anti-CoREST antibody as described in FIG. 12F and Western blots were developed with anti-Nurr1 antibody. Equal loading was confirmed by re-blotting with anti-CoREST antibody. (FIG. 13C) siRNA against NLK reverts Nurr1-mediated repression of iNOS-promoter activity. iNOS-luciferase reporter assay in RAW264.7 cells with siRNAs against indicated molecules or control was performed as described in FIG. 13A. *, p<0.01, **, p<0.001 compared to Nurr1 with control siRNA. (FIG. 13D) NLK phosphorylates Nurr1 in vitro. NLK in vitro kinase assay was performed as described in Examples. Arrows indicate phosphorylated GST-Nurr1 and autophosphorylation of NLK. The migration position of GST-CoREST is indicated by an asterisk. GST substrates are shown in FIG. 23D. (FIG. 13E) NLK is required for physical interaction of Nurr1 and CoREST. BV2 cells were transfected with siRNA against NLK or control. Co-IP of Nurr1 and CoREST was performed as described in FIG. 13B. (FIG. 13F) Recruitment of Nurr1, CoREST and p65 to the iNOS promoter in BV2 cells shown by ChIP assay. Data represent fold enrichment of iNOS-promoter precipitated by the indicated antibodies compared to control IgG as determined by qPCR. (FIG. 13G) Nurr1 is recruited to the iNOS-promoter in the SN after LPS stimulation as documented by ChIP assay. Data are shown as averages of fold enrichment against control IgG and SD. (FIG. 13H) Nurr1-dependent recruitment of CoREST to TNFα-promoter and clearance of p65 from the TNFα-promoter. ChIP assay was performed in shNurr1- or shControl-BV2 cells and data are shown as fold enrichment over control IgG of TNFα-promoter precipitated with antibodies against CoREST (left panel) or p65 (right panel).

FIGS. 14A-I show Nurr1 suppression of inflammatory mediators in murine astrocytes. (FIGS. 14A-B) IL1R1 (FIG. 14A) and p55TNFR (FIG. 14B) are predominantly expressed on astrocytes as determined by qPCR assay of mRNA extracted from mouse primary microglia and astrocytes. *, p<0.01. (FIG. 14C) Astrocytes are more responsive to IL1β and TNFα stimulation compared to microglia. Primary mouse microglia and astrocytes were stimulated with 20 ng/ml TNFα or 10 ng/ml IL1β for 6 h. iNOS mRNA level was determined by qPCR as described before. (FIG. 14D) Nurr1 mRNA is upregulated by inflammatory stimuli in astrocytes. Mouse primary astrocytes were stimulated with 20 ng/ml TNFα or 10 ng/ml IL1β for the indicated time and mRNA extraction and qPCR were performed as described before. (FIGS. 14E-I) Knockdown of Nurr1 in astrocytes increases mRNA of neurotoxic mediators. Mouse primary astrocytes were infected with shCtrl- or shNurr1-lentivirus and cells were stimulated with 20 ng/ml TNFα or 10 ng/ml IL1β for 6 h. iNOS (FIG. 14E), Ncf1 (FIG. 14G), CSF1 (FIG. 14H) and BDNF (FIG. 14I) mRNA expressions were determined by qPCR. (FIG. 14F) Increased NO production in Nurr1-knockdown astrocytes. NO production in astrocytes stimulated with TNFα (gray bar) or IL1β (black bar) was measured by Griess reaction.

FIGS. 15A-I show the CoREST complex requirement for Nurr1-mediated repression in astrocytes. (FIG. 15A) IL1β stimulation enhances physical association of Nurr1 and p65 in mouse primary astrocytes. Lysates of astrocytes stimulated with IL1β (10 ng/ml) for the indicated times were immunoprecipitated with anti-Nurr1 antibody and Western blots were developed with anti-p65 antibody. Equal loading was determined as described in FIG. 12F. (FIG. 15B) Recruitment of Nurr1 and p65 to iNOS-promoter in mouse primary astrocyte shown by ChIP assay. Data represent fold enrichment of iNOS-promoter precipitated with the indicated antibodies compared to control IgG as determined by qPCR. (FIG. 15C) Altered recruitment of Nurr1 to iNOS-promoter in the presence of GSK3β-specific inhibitor SB216763 (SB21). Mouse primary astrocytes were treated with SB21 as described in FIG. 12A. Data represent fold enrichment of iNOS-promoter precipitated with antibody against Nurr1 compared to control IgG as determined by qPCR. (FIG. 15D) Physical interaction of Nurr1 and CoREST in mouse primary astrocytes. Co-IP was performed with anti-Nurr1 antibody and Western blots developed with anti-CoREST antibody. The membranes were stripped and equal loading was confirmed by re-blotting with anti-Nurr1 antibody. (FIG. 15E) Recruitment of Nurr1 and CoREST to iNOS-promoter in mouse primary astrocytes shown by ChIP assay. Data represent fold enrichment of iNOS-promoter precipitated with the indicated antibodies compared to control IgG as determined by qPCR. (FIGS. 15F-H) Knockdown of the components of CoREST-repressor complex increases mRNA of inflammatory mediators. Mouse primary astrocytes were infected with lentivirus carrying shRNA against CoREST, LSD1, G9a, HDAC1 or control. Cells were stimulated with 10 ng/ml IL1β for 6 h and mRNA expression of iNOS (FIG. 15F), CSF1 (FIG. 15G) and Ncf1 (FIG. 15H) was determined by qPCR. I. Nurr1-dependent clearance of p65 from iNOS promoter. ChIP assay was performed in shNurr1- or shCtrl-astrocyte and data shown as fold enrichment over control IgG of iNOS promoter precipitated with antibody against p65.

FIG. 16 shows Nurr1 functioning to inhibit neurotoxic gene expression in microglia and astrocytes via a CoREST-dependent transrepression pathway. Upper panel shows a model for communication among microglia, astrocyte and neurons. Lower panel shows a model for Nurr1/CoREST-mediated repression.

FIGS. 17A-F show Nurr1 mRNA and protein expression in microglial cells and astrocytes. (FIG. 17A) Expression of Nurr1 protein in microglia and astrocytes was determined by immunocytochemistry. Primary mouse microglia (upper panels) and astrocytes (lower panels) were co-labeled with αNurr1 antibody and aMac2 for microglia or anti-GFAP for astrocytes, respectively. Nuclei were labeled with DAPI. (FIG. 17B) Nurr1 protein is expressed in resting microglia and astrocytes. Nuclear extracts from primary mouse microglia (M) stimulated with LPS for 12 hours (+) or without stimulation (−) and primary mouse astrocytes (A) stimulated with IL1β for 12 hours (+) or without stimulation (−) were used for the determination of Nurr1 protein expression by western-blotting. Equal protein loading was checked by anti-actin western-blotting. (FIG. 17C) Mouse primary microglia were stimulated with LPS for the indicated times (hrs). mRNA expression of Nurr1 was determined by qPCR and normalized against HPRT expression. (FIG. 17D) Human primary microglia were stimulated with LPS for the indicated times (hrs). mRNA expression of Nurr1 was determined by qPCR as described in A. (FIG. 17E) BV2 murine microglia cells were stimulated with LPS for 6 h and mRNA expression of Nurr1 was determined as described in FIG. 17C. (FIG. 17F) Injection of LPS into SN increased the mRNA expression of Nurr1 determined by qPCR. The SN was microdissected from the mouse brain as described in Examples. The diagram of the experiment is shown at the top. *, p<0.05 and **, p<0.01 compared to no treatment (no TX).

FIGS. 18A-H show decreased Nurr1 expression in the brain enhances inflammation. (FIG. 18A) A cartoon indicating the site of lentiviral and LPS injection into the SN at AP 3.3 mm. (FIG. 18B) Validation of knockdown efficiency in SN samples. Microdissected SN samples were isolated from mice injected with lentivirus followed by PBS or LPS. Expression of Nurr1 was determined by qPCR as described before. *, p<0.01 compared to shCtrl. (FIGS. 18C-D) The expression of TH (FIG. 18C) and CD11b (FIG. 18D) in SN samples was determined by qPCR as described before. (FIG. 18E) Nurr1 protein expression in F4/80+ microglia in SN samples from shCtrl/LPS injected group (upper panel) and shNurr1/LPS injected group (lower panel). Scalebar 10 μM for all images. (FIG. 18F) The expression of Cleaved caspase-3 in SN samples injected with shNurr1 followed by LPS, including TH, Caspase-3, and DAPI. (FIG. 18G) Atypical TH+ neurons (top right, indicated by arrows) were found close to the activated microglia determined by morphology (top left, indicated by arrow) including TH, Iba-1, and DAPI. Scale bars (20 μm). See Examples for the explanation of normal/pathological TH+ neurons. (FIG. 18H) Infection of lentivirus in different cell types in SN. Virus-infected cells (GFP+) were co-labeled with markers for TH (top panel), GFAP (middle panel) and Iba-1 (bottom panel). The brain section from the mice injected with shNurr1-lentivirus followed by PBS (left column) or LPS (right column) are shown. Arrows indicate double-positive cells.

FIGS. 19A-C show depletion of Nurr1 resulting in enhancement of A30P α-Synuclein-mediated inflammation and the loss of TH+ neurons in the SN. (FIG. 19A) mRNA expression of IL1β (left) and TNFα (right) mRNA in shNurr1 and shCtrl SN after injection of a lentivirus directing expression of α-Synuclein (A30P) mutant (n=2). mRNA extraction and qPCR were performed as described before. Error bars represent SD. Asterisk, p<0.01 compared to shCtrl/A30P-injected. (FIG. 19b) Fluorescent-TH staining of a representative brain section of mouse SN injected with shCtrl- or shNurr1-lentivirus and A30P α-Synuclein is shown at AP −3.3 mm (n=4). An overview of the experimental scheme is shown in the top panel. (FIG. 19C) Stereological analysis of TH+ cell numbers in the shNurr1 groups compared to the shCtrl-injected and the uninjected sides. Ten days after the first injection, the histology and stereological analysis were performed as described before. Data are shown as an average±SD and * indicates p<0.01 compared to shCtrl-injected group.

FIGS. 20A-G show expression of molecules related to TLR4-signaling. (FIGS. 20A-E) The expression of TLR4 (FIG. 20A), CD14 (FIG. 20B), MD2 (FIG. 20C), MyD88 (FIG. 20D) and TRIF (FIG. 20E) compared to HPRT was determined in mouse or human primary microglia, primary astrocytes and neuronal cell lines, Neuro2A (mouse) and SK—N—SH cells (human) by qPCR as described before. *, p<0.01 compared to expression in microglia cells. (FIG. 20F) mRNA expression of TNFα in primary human microglia, primary human astrocytes or SK—N—SH cells upon LPS stimulation. Cells were stimulated with LPS for indicated times and qPCR was performed as described before. (FIG. 20G) The protein expression of cleaved caspase-3 after the treatment of TNFα plus CHX and LPS for indicated times in Neuro2A cells.

FIG. 21 shows Nurr1 control of various pro-inflammatory mediators. Primary microglial cells were transfected with validated siRNAs against Nurr1 or non-targeting control. After 48 h, cells were stimulated with LPS and the endogenous mRNA expression of indicated pro-inflammatory mediators normalized against HPRT was determined by qPCR. Error bars represent SD. *, p<0.01 compared to unstimulated control siRNA transfected sample and **, p<0.01 compared to LPS-stimulated control siRNA transfected sample.

FIGS. 22A-I show that Nurr1 is SUMOylated and acts as a transrepressor of pro-inflammatory mediators as a monomer. (FIGS. 22A-C) The secretion of TNFα (FIG. 22A) and IL1β (FIG. 22B) and NO production (FIG. 22C) in response to LPS in shCtrl- and shNurr-BV2 cells. BV2 cells were stimulated with LPS for 24 h. Supernatant was harvested and secretion of TNFα and IL1β was determined by ELISA. NO production was measured by Greiss reaction. Data are shown as an average of biological triplicates and SD. (FIG. 22D) Each mutant construct shown in FIG. 12B was tested for activity using a promoter under the control of a Nurr1 monomer binding site (NBRE-luciferase). *, p<0.01 compared to mock. (FIG. 22E) The effect of knockdown of PIAS4 in Nurr1-mediated repression of the iNOS-promoter. (FIG. 22F) SUMOylation assay with PIAS4 or IL1β stimulation. Western blots were incubated with anti-Flag antibody to detect SUMOylated Nurr1. (FIG. 22G) The effect of K558R and K576R in Nurr1/RXR heterodimer reporter (DR5-luciferase). Mutants or wild-type Nurr1 were transfected into RAW264.7 cells. DR5-luciferase and NBRE-luciferase reporter assays were performed as described before. *, p<0.01 compared to FL. (FIG. 22H) mRNA expression of TNFα in shNurr1-2-BV2 cells reconstitution with a non-targeted (NT) form of WT Nurr1 and SUMO mutants of Nurr1 (K558R and K576R). Expression of endogenous TNFα mRNA is presented under each treatment condition relative to levels in untreated BV2 cells transduced with control shRNA and mock Nurr1 lentivirus. *, p<0.01 compared to mock control cells. (FIG. 22I) Levels of wild type and mutant forms of Nurr1 in BV2 cells transduced with Ctrl shRNA (Ctrl) or Nurr1 shRNA-2 (sh2) and then infected with mock lentivirus (Mock), or lentiviruses directing expression of nontargeted Nurr1 (Nurr1-NT) of wild-type (WT) or SUMO mutants (K558R and K576R) as indicated.

FIGS. 23A-E show Nurr1-mediated repression is GSK3β-dependent. (FIG. 23A) Protein expression levels of Nurr1, p65 and actin in nuclei from BV2 cells treated with LPS for the indicated time were shown as input controls for FIG. 12F. (FIG. 23B) The effect of overexpression of GSK3β-K85R kinase-dead mutant (GSK3β-KD) in Nurr1-mediated repression of TNFα luciferase assay. TNFα-Luciferase assay was performed as described in FIG. 12B with indicated amounts of GSK3β-KD transfection. (FIG. 23C) The effect of phosphorylation of S468 in p65 to the binding to Nurr1 in vitro. GST-pull down assay was performed with ³⁵S-labeled-p65 to the GST-Nurr1 or p65-S468A mutant in the presence of active GSK3β. (FIG. 23D) The effect of GSK3β-specific inhibitor (SB21) to the protein expression of Nurr1 and CoREST. BV2 cells were treated with SB21 at indicated concentrations followed by LPS stimulation. The expression of Nurr1 and CoREST was determined by Western blotting with antibodies against each protein. Equal loading was tested by Western blotting with anti-actin antibody. The effect of SB21 was tested by the decrease of phosphorylation of p65 at 5468 by Western blotting. The membrane was stripped and equal loading was verified by Western blotting with αp65 antibody. (FIG. 23E) The effect of the overexpression of p65 and phosphorylation-deficient mutant p65 (S468A) was tested by transfection into RAW cells and iNOS-luciferase activity was measured as described before.

FIGS. 24A-G show the Nurr1 requirement for CoREST and its complex for repressor function. (FIG. 24A) Data for the experiment shown in FIG. 13A are replotted as relative luciferase activity (rel. luciferase unit) for unstimulated (−) and LPS-stimulated (LPS) cells in the presence of the indicated siRNAs. (FIG. 24B) The effect of knockdown of CoREST repressor-complex components LSD1, G9a and HDAC1 in Nurr1-mediated repression in iNOS-luciferase activity. Nurr1-expression vector or control vector and siRNA against indicated molecules were transfected into RAW264.7 cells. iNOS-promoter activity was measured as described before. (FIG. 24C) The protein expression of Nurr1 and CoREST in BV2 cells after stimulation with LPS as a control for FIG. 13B. (FIG. 24D) GST-pull down assay with ³⁵S-labeled CoREST to GST-Nurr1 (left panel) and ³⁵S-labeled Nurr1 to GST-CoREST (right panel). GST-pull down assay was performed as described in Examples. Both GST-fusion proteins were visualized by CBB staining (FIG. 24E) The interaction of Nurr1 DNA-binding domain (DBD) and CoREST. GST-pull down was performed as described before. (FIG. 24F) The effect of the overexpression of Nurr1-DBD in the interaction between Nurr1 and CoREST. Hela cells were transfected with Flag-tagged Nurr1 (Flag-Nurr1) and HA-tagged CoREST (HA-CoREST) with or without Myc-tagged Nurr1-DBD (Myc-DBD). IP was performed with anti-HA beads and Western blotting was developed with anti-Flag antibody. (FIG. 24G) Nurr1-mediated repression of iNOS-promoter in the presence of the overexpression of Nurr1-DBD. iNOS-luciferase assay was performed as described before.

FIGS. 25A-E show the role of NLK and recruitment of Nurr1, CoREST and HDAC1 to target promoters. (FIG. 25A) Effect of NLK knockdown in the iNOS-luciferase assay is shown as a control for FIG. 13C. (FIG. 25b) The effect of NLK kinase-dead mutant K155M (NLK-KD) in Nurr1-mediated transrepression of the iNOS-promoter activity. The indicated amounts of NLK-KD and Nurr1 expression vector were transfected into RAW246.7 cells. iNOS-luciferase assay was performed as described before. (FIG. 25C) Recruitment of Nurr1, CoREST and p65 to the TNFα promoter in BV2 cells shown by ChIP assay. Data represent fold enrichment of iNOS-promoter precipitated by the indicated antibodies compared to control IgG as determined by qPCR. (FIG. 25D) The recruitment of Nurr1 to the TNFα-promoter in the SN after LPS stimulation. Data are shown as averages of fold enrichment against control IgG and SD. (FIG. 25E) The recruitment of HDAC1 to the target gene promoters in the absence of Nurr1 is shown. shCtrl or shNurr1-BV2 cells were stimulated with LPS for the indicated time and ChIP assay using anti-HDAC1 antibody was performed as described in Examples.

FIGS. 26A-B show Nurr1/CoREST repressor complex inhibition of the production of neurotoxic factors in microglia. (FIG. 26A) Validated siRNAs against molecules involved in the Nurr1/CoREST pathway were transfected in BV2 cells. CMs were harvested from transfected cells as described in FIG. 11G and effects on viability of Neuro2A cells were determined using a TUNEL ELISA assay. (FIG. 26B) Protein expression levels of Nurr1, p65 and actin in the nuclei from mouse primary astrocytes stimulated with 10 ng/ml IL1β for the indicated time were determined by Western blotting. Data were shown as input controls for FIG. 15A.

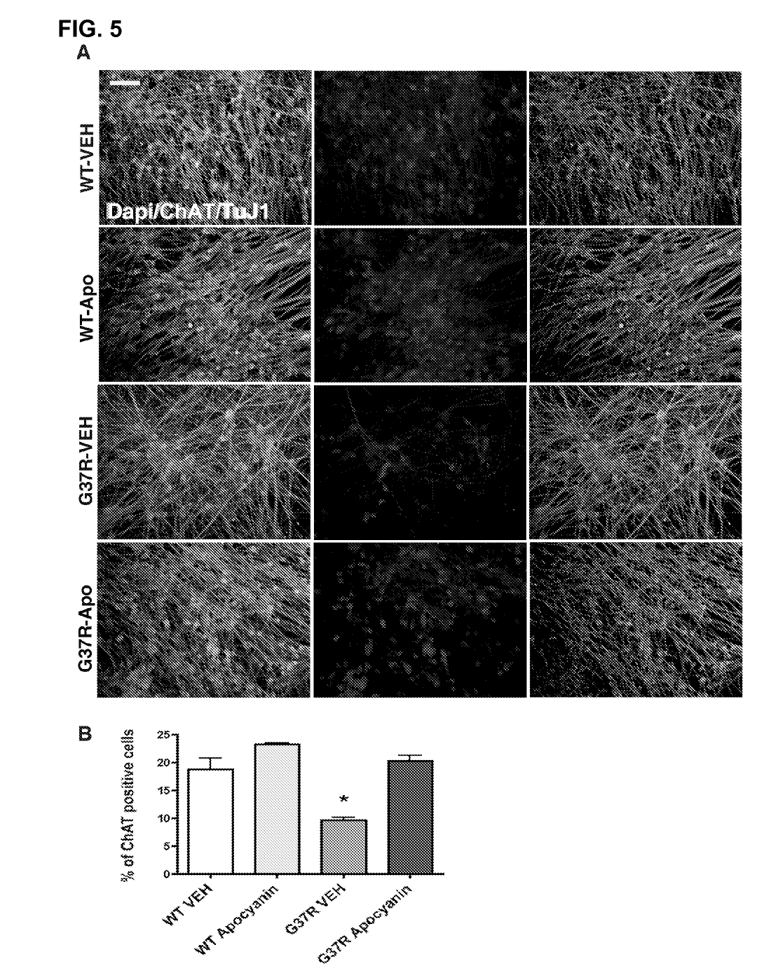

FIGS. 27A-H show Nurr1 suppression of inflammatory mediators in human astrocytes. (FIGS. 27A-B) Nurr1 acts as a repressor of neurotoxic factors in human primary astrocytes, in validation of FIG. 5. The expression of IL1R1 (FIG. 27A) and p55TNFR (FIG. 27B) in astrocytes. mRNA was extracted from human primary microglia and astrocyte and qPCRs were performed as described in Examples. *, p<0.01. (FIG. 27C) mRNA expression of iNOS in astrocytes to the response to the IL1β and TNFα stimulation compared to microglia were determined by qPCR. Primary human microglia and astrocytes were stimulated with TNFα and IL1β for 6 h and qPCR was performed as described before. (FIG. 27D) The expression of Nurr1 in astrocytes after inflammatory stimuli. Human primary astrocytes were stimulated with TNFα and IL1β for the indicated time and mRNA extraction and qPCR were performed as described before. (FIGS. 27E-H) The effect of the knockdown of Nurr1 in human astrocytes. Human primary astrocytes were infected with shCtrl- or shNurr1-lentivirus and cells were stimulated with TNFα and IL1β for 6 h. iNOS (FIG. 27E), Ncf1 (FIG. 27F), CSF1 (FIG. 27G) and BDNF (FIG. 27H) mRNA expression was determined by qPCR as described before.

FIGS. 28A-B illustrate knockdown efficiency. (FIG. 28A) Effects of siRNAs and shRNAs used in this study were verified by Western blotting or qPCR for their respective targets. (FIG. 28B) Hyperactivation of shNurr1-BV2 cells is rescued by reconstitution with non-targeted (NT) wild type or mutant forms of Nurr1 that are not recognized by shRNA-1 (sh1) or shRNA-2 (sh2), respectively. Protein levels of Nurr1 in each cell type are shown in bottom panel.

FIGS. 29A-B show Nurr1 suppression of inflammatory mediators in human microglia. Human primary microglia cells were infected with lentivirus encoding shRNA against Nurr1 (shNurr1) or scramble control (shCtrl). Two days after the infection, cells were stimulated with 0.1 μg/ml LPS for 6 hours (black column) or untreated (white column) and normalized mRNA expression against HPRT of TNFα (FIG. 29A) and iNOS (FIG. 29B) were determined by quantitative PCR (qPCR).

FIGS. 30A-H show Nurr1 suppression of inflammatory mediators in human astrocytes. (FIGS. 30A-B). The expression of IL1R1 (FIG. 30A) and p55TNFR (FIG. 30B) in astrocytes. mRNA was extracted from human primary microglia and astrocyte and qPCRs were performed as described in Example 2 (*p<0.01). (FIG. 30C) mRNA expression of iNOS in astrocytes to the response to the IL1β and TNFα stimulation compared to microglia were determined by qPCR. Primary human microglia and astrocytes were stimulated with TNFα and IL1β for 6 h and qPCR was performed as described in Example 2. (FIG. 30D) The expression of Nurr1 in astrocytes after inflammatory stimuli. Human primary astrocytes were stimulated with TNFα and IL1β for the indicated time and mRNA extraction and qPCR were performed as described in Example 2. (FIGS. 30E-H). The effect of the knockdown of Nurr1 in human astrocytes. Human primary astrocytes were infected with shCtrl- or shNurr1-lentivirus and cells were stimulated with TNFα and IL1β for 6 h. iNOS (FIG. 30E), Ncf1 (FIG. 30F), CSF1 (FIG. 30G) and BDNF (FIG. 30H). mRNA expression was determined by qPCR as described in Example 2.

FIGS. 31A-F show indazol-estrogen suppression of the inflammation in human primary microglia and astrocytes. Primary human microglia (FIGS. 31A-C) and primary human atrocities (FIGS. 31D-E) were treated with Indazol-Estrogen-Bromide (Br), Indazol-Estrogen-Chloride (Cl), 17β-Estradiol (E2) or vehicle (ethanol:EtOH) for 1 hour followed by 0.1 mg/ml LPS stimulation for 6 hours (black column: 6 hr) or no LPS treatment (white column: Ohr). Normalized mRNA expression against HPRT of IL1β (FIG. 31A), IL23p19 (FIG. 31B), TGFβ (FIG. 31C) in microglia cells and BAFF (FIG. 31D), IL23p19 (FIG. 31E), iNOS (FIG. 31F) in astrocytes were determined using quantitative PCR.

### DETAILED DESCRIPTION OF THE INVENTION

### I. Definitions

A “test agent,” as used herein, is a chemical or biological agent that is tested using the methods provided herein.

A “chemical or biological agent,” as used herein, refers to a chemical compound or biological molecule or agents that include a chemical compound component of biological compound component. Chemical compounds and biological molecules include, for example, synthetic small molecule modulators, peptides and proteins (e.g. antibodies and fragments thereof), saccharides and polysaccharides and derivatives thereof, nucleic acids, and the like.

The terms “treating” or “treatment” refers to any indicia of success in the treatment, prevention, or amelioration of an injury, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation.

An “effective amount” is an amount of a kinase inhibitor sufficient to contribute to the treatment, prevention, or reduction of a symptom or symptoms of a disease, or to inhibit the activity or a protein kinase relative to the absence of the kinase inhibitor. Where recited in reference to a disease treatment, an “effective amount” may also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) a disease, or reducing the likelihood of the onset (or reoccurrence) of a disease or its symptoms. The full prophylactic effect does not necessarily occur by administration of one dose, and may occur only after administration of a series of doses. Thus, a prophylactically effective amount may be administered in one or more administrations.

“Nucleic acid” refers to deoxyribonucleotides or ribonucleotides and polymers thereof in single- or double-stranded form, or complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs). Nucleic acids also include complementary nucleic acids.

“Polypeptide” refers to a polymer in which the monomers are amino acids and are joined together through amide bonds, alternatively referred to as a “peptide.” The terms “peptide” and “polypeptide” encompass proteins. Unnatural amino acids, for example, β-alanine, phenylglycine and homoarginine are also included under this definition. Amino acids that are not gene-encoded may also be used in the present invention. Furthermore, amino acids that have been modified to include reactive groups may also be used in the invention. All of the amino acids used in the present invention may be either the D- or L-isomer. The L-isomers are generally preferred. In addition, other peptidomimetics are also useful in the present invention. For a general review, see, Spatola, A. F., in CHEMISTRY AND BIOCHEMISTRY OF AMINO ACIDS, PEPTIDES AND PROTEINS, B. Weinstein, eds., Marcel Dekker, New York, p. 267 (1983).

### II. Methods

It has been discovered that the pro-inflammatory activity of human glial cells results in damage to human neuron cells. Damage to human neuron cells are known to be linked with various disease states, such as Parkinson's disease (PD) and Amyotrophic Lateral Sclerosis (ALS). Provided herein are methods (e.g. assays, tests, screens) useful in identifying one or more neuroprotective agents. A “neuroprotective agent,” as used herein, refers to a chemical or biological agent capable of reducing the pro-inflammatory activity of pro-inflammatory human glial cells. “Pro-inflammatory activity” of a pro-inflammatory human glial cell, as used herein, refers to the activity of a human glial cell resulting in the production or expression of known members of a human glial cell inflammatory response process. Known members of the human glial cell inflammatory response process include, but not limited to, reactive species of oxygen (ROS), neurosecretory protein Chromogranin A, secretory cofactor cystatin C, NADPH oxidase, nitric oxide synthase enzymes (such as iNOS), TNFα, IL-1β, and NF-κB-dependent inflammatory response proteins.

In one aspect, a method is provided for determining whether a test agent is a neuroprotective agent (e.g. to identify neuroprotective agents, to assay for neuroprotective agents, to screen for neuroprotective agents, etc.). The method includes adding a test agent to a cellular culture (e.g. a plurality of pro-inflammatory human glial cells). The cellular culture includes pro-inflammatory human glial cells. A level of pro-inflammatory activity of the pro-inflammatory human glial cells is determined in the presence of the test agent. The level of pro-inflammatory activity of the pro-inflammatory human glial cells in the presence of the test agent is compared to a control thereby determining whether the test agent is a neuroprotective agent. In some embodiments, a lower level of pro-inflammatory activity of the pro-inflammatory human glial cells in the presence of the test agent compared to a control is indicative of the test agent being a neuroprotective agent.

In some embodiments, the methods provided herein further include contacting the pro-inflammatory human glial cell with an inflammation inducing agent. The purpose of the inflammation inducing agent is to elicit or induce an inflammatory response from the human glial cell in order to optimize test conditions. Any appropriate inflammation inducing agent may be employed, such as bacterial lipopolysaccharide (LPS), TNFα, rotenone, or expression of toxic proteins, such as mutated superoxide dismutase1 (SOD1) or mutated forms of α-synuclein.

Any appropriate control may be used to compare the level of pro-inflammatory activity of the pro-inflammatory human glial cells in the presence of the test agent. A person having ordinary skill in the art, using the guidance provided herein and the background knowledge in the art, would immediately understand what appropriate controls may be employed. The control is typically a level of pro-inflammatory activity of the pro-inflammatory human glial cells determined using all the same experimental elements used in determining the level pro-inflammatory activity in the presence of the test agent, with the exception that the test agent is not present. The test agent may be simply absent, or may be replaced with a control agent (i.e. an agent known to produce a particular level of pro-inflammatory activity or no pro-inflammatory activity). Thus, in some embodiments, the control is a level of pro-inflammatory activity of the pro-inflammatory human glial cells in the absence of the test agent.

The level of pro-inflammatory activity of the pro-inflammatory human glial cells may be determined using any appropriate technique, including those techniques described herein. For example, pro-inflammatory activity may be assessed by measuring the amount, production or expression of known members of the human glial cell inflammatory response process (e.g. reactive species of oxygen (ROS), neurosecretory protein Chromogranin A, secretory cofactor cystatin C, NADPH oxidase, nitric oxide synthase enzymes (such as iNOS), TNFα, IL-1β, and NF-κB-dependent). Moreover, it has been discovered herein that pro-inflammatory activity of human glial cells results in damage to human neuron cells. Consequently, pro-inflammatory activity may be assessed by measuring an amount of damage to human neuron cells in the presence of pro-inflammatory human glial cells (e.g. where the cellular culture further includes human neuron cells to from a cellular co-culture of human neuron cells and pro-inflammatory human glial cells).

Thus, in some embodiments, the level of pro-inflammatory activity of the pro-inflammatory human glial cells is determined by measuring an amount of soluble inflammatory factors produced by the pro-inflammatory human glial cells. The level of pro-inflammatory activity of the pro-inflammatory human glial cells may also be determined by measuring an amount of expression, an amount of activity, or the number of pro-inflammatory proteins expressed by the pro-inflammatory human glial cells. The level of pro-inflammatory activity of the pro-inflammatory human glial cells may also be determined by measuring an amount of transcription of a gene encoding a pro-inflammatory protein within the pro-inflammatory human glial cells (e.g. using quantitative PCR to determine the amount of mRNA).

In some embodiments, the cellular culture further comprises human neuron cells. Thus, the level of pro-inflammatory activity of the pro-inflammatory human glial cells may be determined by determining an amount of human neuron cells damaged by the pro-inflammatory activity of the pro-inflammatory human glial cell. Any appropriate method may be used to determine whether human neuron cells are damaged by the pro-inflammatory activity of the pro-inflammatory human glial cell. Appropriate methods include, for example, determining the number of viable (e.g. surviving, reproducing, growing, etc.) human neuron cells before and after exposure to the pro-inflammatory activity of the pro-inflammatory human glial cell. A decrease in the number of viable human neuron cells after exposure to the pro-inflammatory activity provides a quantitative measure of an amount of human neuron cells damaged by the pro-inflammatory activity. Thus, in some embodiments, the amount of human neuron cells damaged by the pro-inflammatory activity in the absence or presence of the test agent is determined by determining an amount of human neuron cells killed by the pro-inflammatory activity. And an amount of human neuron cells damaged by the pro-inflammatory activity in the absence or presence of the test agent may also be determined by determining an amount of human neuron cells surviving the pro-inflammatory activity. Other methods of determining whether human neuron cells are damaged by the pro-inflammatory activity may be used, such as detecting the presence (e.g. production, transcription, expression or secretion) or amount of cellular signals indicative of a damaged cell.

A pro-inflammatory human glial cell is a human glial that has been treated to have increased capacity for pro-inflammatory activity (e.g. greater pro-inflammatory activity than the same human glial that has not been treated). Any appropriate treatment may be employed, including treatment with a chemical or biological agent that inhibits (e.g. suppresses) the activity of an anti-inflammatory cellular component. Genetic engineering or cloning techniques may also be employed to form a human glial cell having a mutant gene encoding an anti-inflammatory cellular component that does not have anti-inflammatory activity (e.g. null mutant or knockout mutant). Likewise, a chemical or biological agent that increases the activity of an pro-inflammatory cellular components may also be employed, and genetic engineering or cloning techniques may be used to form a human glial cell having a mutant gene encoding a pro-inflammatory cellular component with increased pro-inflammatory activity. Typically, the treatment seeks to mimic a known disease state.

Thus, in some embodiments, the pro-inflammatory human glial cells include a nonfunctional anti-inflammatory gene. A “nonfunctional anti-inflammatory gene,” as used herein, refers to an anti-inflammatory gene that produces a reduced amount of a gene product or a gene product with reduced anti-inflammatory activity relative to the amount or activity found in a human glial cell that has not been treated to have an increased capacity for pro-inflammatory activity (i.e. a normal human glial cell). The nonfunctional anti-inflammatory gene may be a mutated anti-inflammatory gene (also referred to herein as a “nonfunctional mutated anti-inflammatory gene”). The nonfunctional anti-inflammatory gene may also be a silenced anti-inflammatory gene. A “silenced anti-inflammatory gene,” as used herein (also referred to herein as a nonfunctional silenced anti-inflammatory gene”), is an anti-inflammatory gene that expresses a reduced amount of anti-inflammatory gene product relative the amount of anti-inflammatory gene product expressed in a normal human glial cell. A silenced nonfunctional anti-inflammatory gene includes knockdowns, knockouts as well as incomplete shut-down of gene expression such as down regulation. In some embodiments, the silenced anti-inflammatory gene is silenced using an antisense nucleic acid. The antisense nucleic acid may be an RNA molecule, such as an interference RNA (RNAi) molecule. Thus, in some embodiments, the silenced anti-inflammatory gene is silenced using a microRNA (miRNA) molecule, small interfering RNA (siRNA) molecule or small hairpin RNA (shRNA) molecule.

In some embodiments, the nonfunctional anti-inflammatory gene is a nonfunctional gene encoding a member (e.g. a mutated member) of the nuclear receptor family of intracellular transcription factors such as the nuclear receptor (NR)4 family of orphan nuclear receptors. Nurr1 (NR4A2) belongs to the nuclear receptor (NR)4 family of orphan nuclear receptors and is known to function as a constitutively active transcription factor by binding to target genes as a monomer or homodimer or as a heterodimer with retinoid X receptors (RXRs) (Aarnisalo et al., 2002; Maira et al., 1999; Wang et al., 2003). In some embodiments, the nonfunctional anti-inflammatory gene is a nonfunctional gene encoding a member of the Nurr family of nuclear receptors (e.g. Nur77, Nor1 or Nurr1 (NR4A2). Thus, in some embodiments, the nonfunctional anti-inflammatory gene is a non-functional NURR gene (or homologue thereof). As discussed above, a non-functional NURR gene is a NURR gene that produces a reduced amount of a Nurr family nuclear receptor or a Nun family nuclear receptor with reduced anti-inflammatory activity relative to the amount or activity found in a normal human glial cell. In some embodiments, the non-functional NURR gene is a non-functional NURR1 gene (or homologue thereof). The non-functional NURR1 gene may be a silenced non-functional NURR1 gene. The silenced non-functional NURR1 gene may be silenced using an shRNA molecule.

In some embodiments, the nonfunctional anti-inflammatory gene is a nonfunctional gene encoding a human superoxide dismutase, such as human superoxide dismutase 1 (SOD1) or homologue thereof. Thus, in some embodiments, the nonfunctional anti-inflammatory gene is a nonfunctional SOD gene, such as a nonfunctional SOD1 gene. The nonfunctional SOD1 gene may be a mutated nonfunctional SOD1 gene. The mutated nonfunctional SOD1 gene may be one of the well known mutations linked to ALS, such as A4V, G37R, G85R or G93A. Thus, in some embodiments, the mutated nonfunctional SOD1 gene is SOD1^{A4V}, SOD1^{G37R}, SOD1^{G85R}, or SOD1^{G93A}. In certain embodiments, the mutated nonfunctional SOD1 gene is SOD1^{G37R}.

In some embodiments, the pro-inflammatory human glial cells are pro-inflammatory human microglial cells or pro-inflammatory human astrocyte cells. In some related embodiments, the pro-inflammatory human microglial cells or pro-inflammatory human astrocyte cells include a nonfunctional anti-inflammatory gene. The anti-inflammatory gene may be a nonfunctional NURR gene (e.g. a nonfunctional NURR1 gene) or a nonfunctional SOD gene (e.g. a nonfunctional SOD1 gene). In certain embodiments, the pro-inflammatory human glial cells are pro-inflammatory human microglial cells and the nonfunctional anti-inflammatory gene is a nonfunctional NURR gene. In some embodiments, the pro-inflammatory human glial cells are pro-inflammatory human astrocyte cells and the nonfunctional anti-inflammatory gene is a nonfunctional SOD1 gene.

In some related embodiments (e.g. where the nonfunctional anti-inflammatory gene is a nonfunctional NURR gene), the level of pro-inflammatory activity of the pro-inflammatory human glial cells is determined by determining an amount of TNFα, induced nitric oxide enzyme (iNOS or NOS2A), or IL-1β produced by the human glial cell, or by determining an amount of expression or activity of an NF-κB-dependent inflammatory response protein. In some further related embodiments, the pro-inflammatory human glial cells are pro-inflammatory human microglial cells.

In other related embodiments, (e.g. where the nonfunctional anti-inflammatory gene is a nonfunctional SOD1 gene) the level of pro-inflammatory activity of the pro-inflammatory human glial cells is determined by determining an amount of reactive species of oxygen (ROS), a neurosecretory protein Chromogranin A (e.g. CHGA), or a secretory cofactor cystatin C (e.g. CC or CST3) produced by the pro-inflammatory human glial cell, or by determining an amount of activity or expression of an NADPH oxidase (e.g. NOX2/gp91^{phox} or CYBB) or an induced nitric oxide synthase enzyme (e.g. iNOS or NOS2A) in the human glial cells. In some further related embodiments, the pro-inflammatory human glial cells are pro-inflammatory human astrocyte cells.

As discussed above, the cellular culture may further include human neuron cells. The human neuron cells may be derived from human embryonic stem cells. In certain embodiments, the human neuronal cells are human motor neuron cells or human dopaminergic neuron cells. Thus, in some embodiments, the level of pro-inflammatory activity of the pro-inflammatory human glial cells is determined by determining an amount of human neuron cells damaged by the pro-inflammatory activity of the pro-inflammatory human glial cell. In related embodiments, the control may be an amount of human neuron cells damaged by the pro-inflammatory activity of the pro-inflammatory human glial cell in the absence of the test agent. As discussed above, the amount of human neuron cells damaged by the pro-inflammatory activity in the absence or presence of the test agent may be determined by determining an amount of human neuron cells killed by the pro-inflammatory activity. And in certain embodiments, the amount of human neuron cells damaged by the pro-inflammatory activity in the absence or presence of the test agent is determined by determining an amount of human neuron cells surviving the pro-inflammatory activity.

In another aspect, a method is provided for determining whether a test agent is a neuroprotective agent. The method includes adding a test agent to a cellular culture comprising pro-inflammatory human astrocyte cells. A level of pro-inflammatory activity of the pro-inflammatory human astrocyte cells in the presence of the test agent is determined. The level of pro-inflammatory activity of the pro-inflammatory human astrocyte cells in the presence of the test agent is compared to a control thereby determining whether the test agent is a neuroprotective agent. The embodiments and description provided in the preceding paragraphs are equally applicable to the method set forth in this paragraph. For example, in some embodiments, the level of pro-inflammatory activity of the pro-inflammatory human astrocyte cells is determined by determining an amount of human motor neuron cells damaged by the pro-inflammatory activity of the pro-inflammatory human astrocyte cell. In certain embodiments, the control is an amount of human motor neuron cells damaged by the pro-inflammatory activity of the pro-inflammatory human astrocyte cell in the absence of the test agent. In some embodiments, the amount of human motor neuron cells damaged by the pro-inflammatory activity in the absence or presence of the test agent is determined by determining an amount of human motor neuron cells killed by the pro-inflammatory activity. The amount of human motor neuron cells damaged by the pro-inflammatory activity in the absence or presence of the test agent may also be determined by determining an amount of human motor neuron cells surviving the pro-inflammatory activity. In some embodiments, the neuroprotective agent is a an agent effective in treating Amyotrophic Lateral Sclerosis.

Any appropriate test agent may be employed in the methods provided herein. In some embodiments, the test agent is an anti-inflammatory agent. An “anti-inflammatory agent” is a chemical or biological agent known to decrease a human cellular inflammation response by decreasing the action of a cellular component that increases a cellular inflammation response or increasing the action of a cellular component that decreases a cellular inflammation response. In some embodiments, the anti-inflammatory agent decreases a human cellular inflammation response mediated by SOD, such as SOD1, or gene products thereof (also referred to herein as an SOD anti-inflammatory agent and SOD1 anti-inflammatory agent, respectively). In some embodiments, the SOD anti-inflammatory agent and SOD1 anti-inflammatory agent decreases a human cellular inflammation response mediated by NOX2. In other embodiments, the anti-inflammatory agent decreases a human cellular inflammation response mediated by NURR, such as NURR1, and gene products thereof (also referred to herein as a NURR anti-inflammatory agent and a NURR1 anti-inflammatory agent, respectively). In some embodiments, the NURR1 anti-inflammatory agent decreases a human cellular inflammation response mediated by CoREST co-repressor complexes to NF-κB target genes. In some embodiments, the test agent is a derivative of apocynin.

In certain embodiments, the test agent is an antioxidant (e.g. reduces the effect of reactive species of oxygen). In other embodiments, the test agent decreases the action or amount of TNFα, IL-1β, an NF-κB-dependent inflammatory response protein, a neurosecretory protein Chromogranin A, a secretory cofactor cystatin C, an NADPH oxidase (e.g. NOX2/gp91^{phox} or CYBB) or an induced nitric oxide synthase enzyme (e.g. iNOS or NOS2A) relative to the absence of the test agent. The test agent may also be an Estrogen Receptor beta (ERβ) binder, such as indazol-estrogen-bromide, indazol-estrogen-chloride, or derivatives thereof.

In another aspect, a method is provided for treating a disease mediated by a human glial cell inflammatory response in a human subject in need thereof. The method includes administering to the human subject an effective amount of an anti-inflammatory agent. The anti-inflammatory agent may be an SOD anti-inflammatory agent such as an SOD1 anti-inflammatory agent. The anti-inflammatory agent may be a NURR anti-inflammatory agent such as a NURR1 anti-inflammatory agent. In certain embodiments, the anti-inflammatory agent is also an antioxidant. In other embodiments, the anti-inflammatory agent decreases the action of TNFα, IL-1β, an NF-κB-dependent inflammatory response protein, a neurosecretory protein Chromogranin A (e.g. CHGA), a secretory cofactor cystatin C (e.g. CC or CST3), an NADPH oxidase (e.g. NOX2/gp91^{phox} or CYBB) or an induced nitric oxide synthase enzyme (e.g. iNOS or NOS2A). The anti-inflammatory agent may also be an Estrogen Receptor beta (ERβ) binder, such as indazol-estrogen-bromide, indazol-estrogen-chloride, or derivatives thereof. In some embodiments, the anti-inflammatory agent is apocynin or a derivative thereof.

The disease mediated by a human glial cell inflammatory response may be Parkinson's disease, schizophrenia, manic depression, rheumatoid arthritis, multiple sclerosis or ALS. In some embodiments, the disease mediated by a human glial cell inflammatory response is Parkinson's disease. In other embodiments, the disease mediated by a human glial cell inflammatory response is ALS. In other embodiments, the disease mediated by a human glial cell inflammatory response is Multiple Sclerosis.

Where the disease is Parkinson's disease, the anti-inflammatory agent may be a NURR anti-inflammatory agent such as a NURR1 anti-inflammatory agent. In some related embodiments, the anti-inflammatory agent decreases the activity or amount of TNFα, induced nitric oxide enzyme (iNOS or NOS2A), IL-1β, or an NF-κB-dependent inflammatory response protein.

Where the disease is ALS, the anti-inflammatory agent may be an SOD anti-inflammatory agent such as an SOD1 anti-inflammatory agent. In some related embodiments, the anti-inflammatory agent decreases the activity or amount of reactive species of oxygen (ROS), a neurosecretory protein Chromogranin A (e.g. CHGA), a secretory cofactor cystatin C (e.g. CC or CST3), an NADPH oxidase (e.g. NOX2/gp91^{phox} or CYBB) or an induced nitric oxide synthase enzyme (e.g. iNOS or NOS2A) in the human glial cells.

### III. Examples

The following examples are provided to illustrate certain particular features and/or embodiments. These examples should not be construed to limit the invention to the particular features or embodiments described. A person having ordinary skill in the art will understand that variations of the particular embodiments may be employed.

### Example 1

### Non-Cell Autonomous Effect on Human SOD1 Mutant Astrocytes on HESC Derived Motor Neurons

### Example 1.1

### Cell Culture Methods

Culture conditions and differentiation of HESC. The HUES cells lines used in this study were HUES9 (Douglas Melton-WiCell) and Cythera 203 (Novocell Inc. San Diego, Calif.). The HESC were differentiated in vitro in motor neurons, adapting the protocol previously described elsewhere (Li et al., 2005). Briefly, the cells were manually dissociated to form embryoid bodies (EBs) and cultured in suspension for 5-6 days. The EBs were then plated in laminin/poli-ornithin coated plates in the presence of a neural induction medium consisting of F12/DMEM (Invitrogen, Carlsbad, Calif.), N2 supplement and 1 μM retinoic acid (RA). The cells started to organize into neural tube-like rosettes and, after 7-8 days in culture, sonic hedgehog (SHH, 500 ng/ml, R&D Systems) and cAMP (1 μM) were added to the culture media for 1 more week. The rosettes were then manually selected using a 10× magnifier (Zeiss) and gently dissociated (by pipetting up and down in a Hanks' enzyme free cell dissociation buffer-Invitrogene). After dissociation, rosettes were pelleted at 1,000 rpm and re-plated either on laminin/poli-ornithine coated coverslips (for direct differentiation) or on top of astrocyte feeder layers for the co-culture experiments. The media was changed for a differentiation medium that consisted of neurobasal medium (Invitrogen), N2 supplement, RA (1 μM), SHH (50 ng/ml) cAMP (1 μM), BDNF, GDNF and IGF (all at 10 ng/ml, Peprotech Inc.). The neurons were cultured in the differentiation media for 3-5 more weeks with or without the astrocyte feeder.

Co-culture of motor neurons and myocyte. C2C12 myoblasts were purchased from ATCC (American Type Culture Collection) and cultured according to the specifications of the manufacturer. After reaching a specific confluence, the myoblasts formed myotubes. The manually dissected rosettes (motor neuron progenitors) were plated on top of the myotubes and the medium was replaced with the differentiation medium (described previously). After 4-6 weeks in co-culture, the cells were fixed and the formation of neuromuscular junctions was observed by incorporation of α-Bungarotoxin conjugated with Alexa 568 (1:200, Molecular Probes, Invitrogen, Carlsbad, Calif.). A population of human neurons in vitro that expressed post-mitotic motor neuron markers, made neuro-muscular junctions, and fired action potentials was consistently generated. Subsequently, the human embryonic stem cell (HESC)-derived motor neurons were co-cultured with human primary astrocytes expressing either the wild type or the mutated form of SOD1 protein (SOD1^{WT} or SOD1^{G37R}, respectively).

Purification and culture of rat primary motor neurons. Primary rat motor neurons were purified following previously published procedures (Arce et al., 1999; Henderson et al., 1993), with some modifications. Briefly, spinal cords were dissected from E14 rat embryos, treated with trypsin (2.5% w/v; final concentration 0.05%) for 10 min at 37° C., and then dissociated. The largest cells were isolated by centrifugation for 15 min at 830 g over a 5.2% Optiprep cushion (Sigma, St Louis, Mo., USA), followed by centrifugation for 10 min at 470 g through a 4% BSA cushion. Purified motor neurons were plated inside 35-mm Petri dishes on 12-mm coverslips previously coated with polyornithine/laminin, and grown 7-10 days in L15 medium with sodium bicarbonate (625 μg/ml), glucose (20 mm), progesterone (2×10⁻⁸ m), sodium selenite, putricine (10⁻⁴ m), insulin (5 μml⁻¹) and penicillin-streptomycin. BDNF (1 ng ml⁻¹), and 2% horse serum were also added to the medium.

Primary astrocyte culture. Human primary astrocytes (HA1800) were obtained from ScienCell Research Laboratories™ (Carlsbad, Calif.) and were cultured according to the providers' guidelines. Briefly, the astrocytes were isolated from fetal human brain (cerebral cortex) and cultured for no more than 15 passages in astrocyte media (AM 1801). The infections were performed in 80% confluent T75 flasks followed by incubation with the lentivirus expressing either the wild type of SOD1 (LV-SOD1^{wT}) or the mutated form of SOD1 (LV-SOD1^{G37R}).

For the co-culture experiments, the astrocytes were plated on laminin/poly-ornithine (Invitrogen and Sigma-Aldrich, St. Louis, Mo.; respectively) coated cover slips 1 day prior to the co-culture. The rosettes were then cultured on top of the astrocytes feeder layer (see Culture Conditions and Differentiation for HESC). Co-cultures were held for 3 weeks.

### Example 1.2

Immunofluorescence. Astrocyte monolayers or astrocyte and motor neuron co-cultures were fixed for 15 minutes with 4% paraformaldehyde in PBS, and immunofluorescence was performed as described previously (Muotri et al., 2005). Briefly, slides were washed with PBS and permeabilized with 0.1% Triton X-100 for 30 minutes and incubated for 2 hours at room temperature in blocking solution (0.1% Triton X-100, 5% donkey serum in PBS). The samples were incubated overnight at 4° C. with primary antibodies diluted in blocking solution, washed in PBS and further incubated for 1 hour at room temperature with secondary antibodies (rabbit, mouse or goat Alexa fluor conjugated antibodies, Molecular Probes-Invitrogen, Carlsbad, Calif.) diluted in blocking solution. The slides were then washed with PBS and mounted. The primary antibodies used were anti-Pax6, anti-Islet 1 and anti-Hb9 (all used at 1:100 and acquired from Developmental Studies Hybridoma Bank, DSHB Iowa City, Iowa), anti-human Nestin (1:200), anti-Olig2 (1:200), anti-ChAt (1:100) and anti-A2B5 (1:500) (all from Chemicon, Temecula, Calif.), anti-TuJ1 and anti-HoxC8 (both 1:200 from Covance Research Products, CA), anti-GFP (Molecular Probes-Invitrogen, CA), anti-GFAP (1:500 from DAKO Carpinteria, Calif.), anti GAD65 (1:200 from Sigma-Aldrich, Mo.)

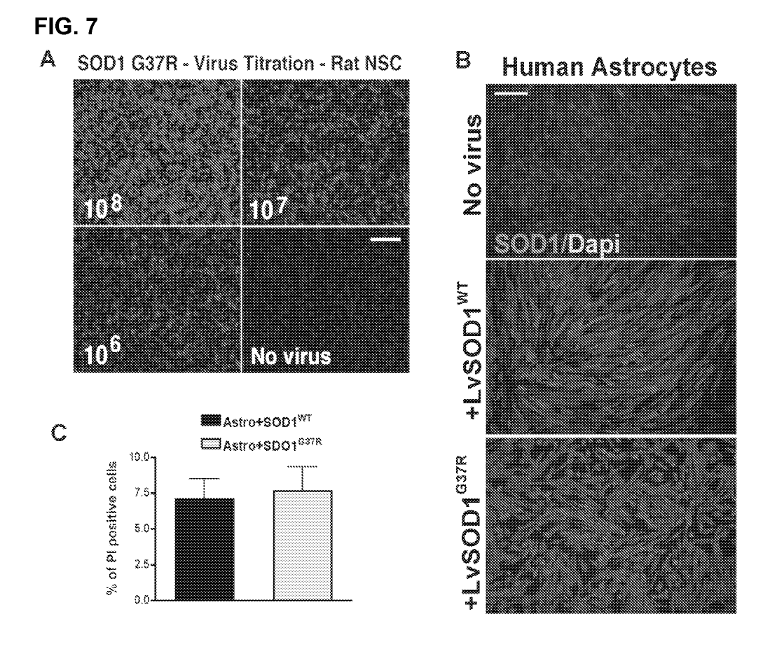

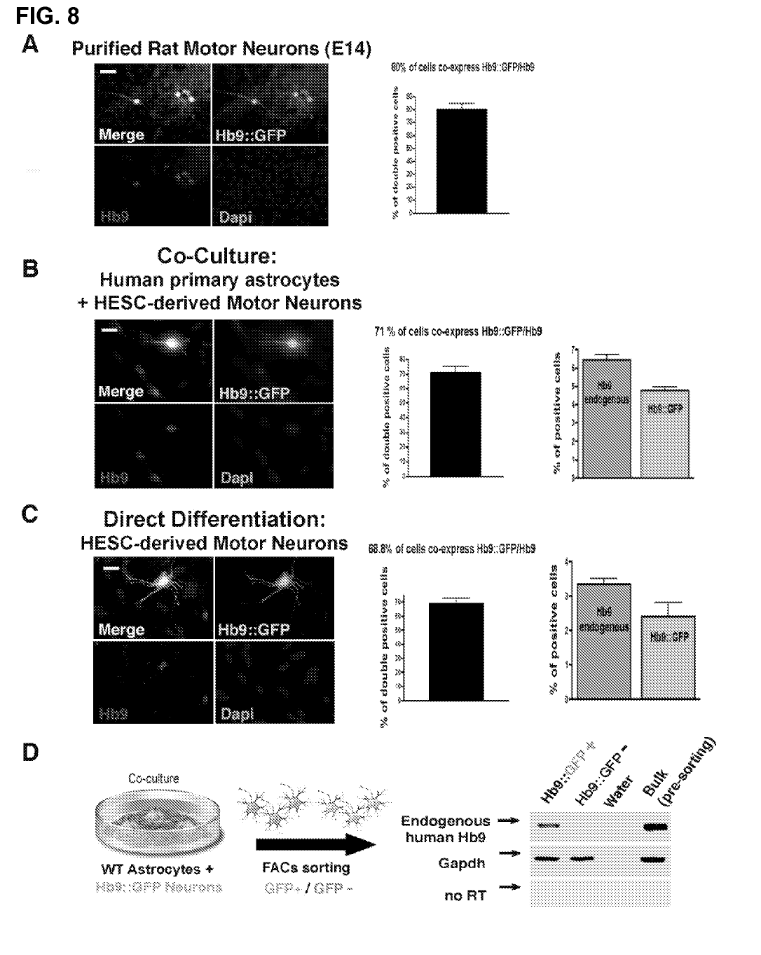

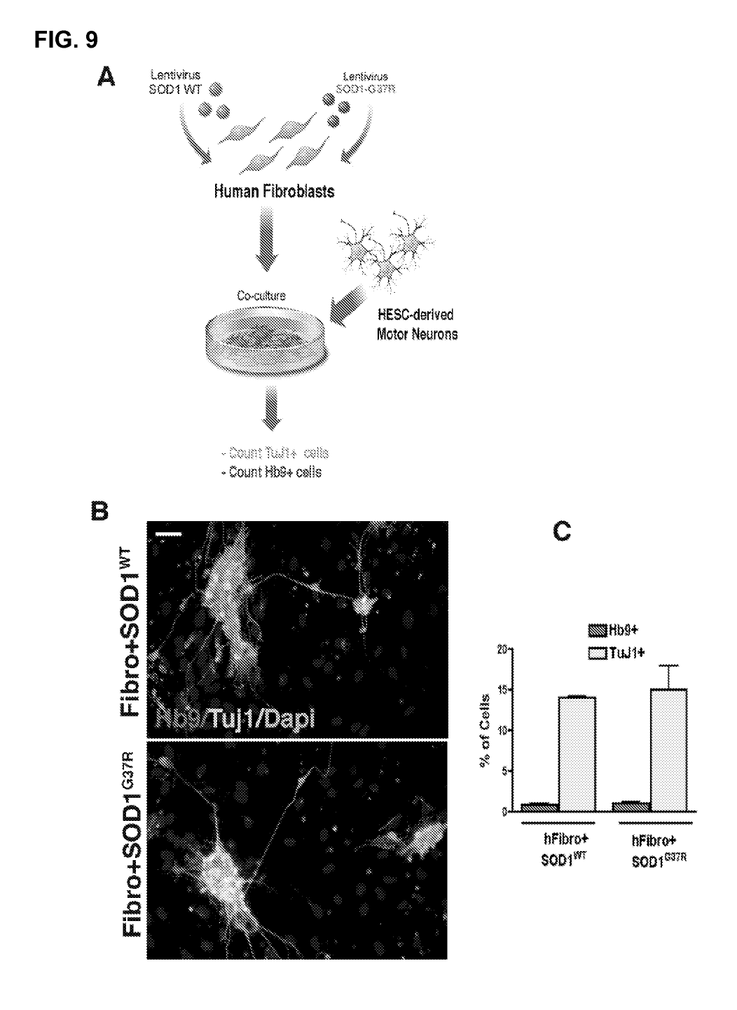

Lentiviral vectors. The viral vectors used in this research were Lenti-SOD1^{WT}, Lenti-SOD1^{G37R}, Lenti-Hb9::GFP, and Lenti-Hb9::RFP (for electrophysiological recordings). Concentrated lentiviral stocks were produced as described (Consiglio et al., 2004). Assessment of virus tittering of Lenti-SOD1^{WT} and Lenti-SOD1^{G37R} was performed in rat neural stem cells (NSC) using an antibody that specifically recognizes human SOD1 protein (1:500, Sigma-Aldrich, St Louis, Mo.; see FIG. 7A) and was estimated as 1×10⁸ units per ml.

Electrophysiology. Whole-cell perforated patch recordings were performed from cultured Hb9::RFP-expressing cells that had differentiated for at least 8 weeks. The recording micropipettes (tip resistance 4-8 MΩ) were tip-filled with internal solution (115 mM K-gluconate, 4 mM NaCl, 1.5 mM MgCl₂, 20 mM HEPES and 0.5 mM EGTA, pH 7.3) and then back-filled with internal solution containing amphotericin B (200 μg/ml). Recordings were made using an Axopatch 200B amplifier (Axon Instruments). Signals were filtered at 2 kHz and sampled at 10 kHz. The whole-cell capacitance was fully compensated, whereas the series resistance was uncompensated but monitored during the experiment by the amplitude of the capacitive current in response to a 5-mV pulse. The bath was constantly perfused with fresh HEPES-buffered saline (115 mM NaCl, 2 mM KCl, 10 mM HEPES, 3 mM CaCl₂, 10 mM glucose and 1.5 mM MgCl₂, pH 7.4). For current-clamp recordings, cells were clamped at −60˜−80 mV. For voltage-clamp recordings, cells were clamped at −70 mV. All recordings were performed at room temperature. Amphotericin B was purchased from Calbiochem. All other chemicals were from Sigma.

RNA isolation and RT-PCR. Total cellular RNA was extracted from ˜5×10⁶ cells using the RNeasy Protect Mini kit (Qiagen, Valencia, Calif.), according to the manufacturer's instructions, and reverse transcribed using the SuperScript III First-Strand Synthesis System RT-PCR from Invitrogen. The cDNA was amplified by PCR using Taq polymerase (Promega, San Luis Obispo, Calif.), and the primer sequences were: hNanog-Fw: 5′ cctatgcctgtgatttgtgg 3′ (SEQ ID NO:1), hNanog-Rv: 5′ ctgggaccttgtcttccttt 3′ (SEQ ID NO:2), hHB9-Fw: 5′ cctaagatgcccgacttcaa 3′ (SEQ ID NO:3), hHB9-Rv: 5′ ttctgtttctccgcttcctg 3′ (SEQ ID NO:4), hChAT-Fw: 5′ actccattcccactgactgtgc 3′ (SEQ ID NO:5), hChAT-Rv: 5′ tccaggcatacaaggcagatg 3′ (SEQ ID NO:6), hGAPDH-Fw: 5′ accacagtccatgccatcac 3′ (SEQ ID NO:7), hGAPDH-Rv: 5′ tccaccaccctgttgctgta 3′ (SEQ ID NO:8). PCR products were separated by electrophoresis on a 2% agarose gel, stained with ethidium bromide and visualized by UV illumination. Product specificity was determined by sequencing the amplified fragments excised from the gel.

Cell Death detection. Cell death was quantified by flow cytometry using 5 μg/mL of propidium iodide (PI) in astrocytes cultures that had been previously infected with LentiSOD1^{WT} or LentiSOD1^{G37R}.

Detection of ROS production. Detection of total cellular ROS was performed using the Image-iT LIVE Green reactive Oxygen Species Detection Kit, according to the manufacturer's directions (Molecular Probes, Invitrogen). Briefly, this assay is based on the principle that the live cell permeable compound, carboxy-H₂DCFDA, emits a bright green fluorescence when it is oxidized in the presence of ROS. The quantification of the ROS production was addressed in 2 ways: 1) counting the number of fluorescent cells and 2) measuring the intensity of the fluorescence emitted by the cells. The relative fluorescence intensity (arbitrary units ranging from 0 to 255, or black to white) was measured in randomly selected fields for each treatment and was analyzed and quantified using ImageProPlus software.

Anti-oxidants treatment. Anti-oxidants stock solutions were diluted in astrocyte media and directly applied to astrocyte monolayers. The cultures were treated for 48 hours prior to reactive species of oxygen (ROS) detection. The compounds used in the experiment were epicatechin (E4018 Sigma Aldrich, 10 μM), luteolin (L9283 Sigma-Aldrich, 5 μM), resveratrol (R5010Sigma-Aldrich, 5 μM), apocynin (178385 Calbiochem, 300 μM), alpha lipoic acid (T5625, Sigma-Aldrich, 50 μg/mL). For neuronal co-cultures, the astrocytes were pre-treated for 48 hours with apocynin and the rosettes were plated on top of them. The co-cultures were carried for 3 more weeks and the medium containing apocynin was replaced three more times during the co-culture period.

Western blotting. Western blotting was carried out using standard protocols. Briefly, total proteins were extracted from astrocyte cultures using 1×RIPA buffer (Upstate, Temecula, Calif.). Protein samples (20 μg) were then separated in 12.5% SDS-PAGE and transferred to nitrocellulose membranes. The membranes were then probed with the following antibodies: mouse anti-actin (1:10,000 Ambion Austin, Tex.), rabbit anti-iNOS (1:1,000), mouse anti-chromogranin A (1:1,000), rabbit anti-cystatin C (1:1,000) and rabbit anti-NOX2 (1:200) all from Abcam (Cambrige, Mass.). Immunoreactive proteins were detected using enhanced chemiluminescence (ECL; Amersham-GE Healthcare, Piscataway, N.J.) and were exposed to X-ray film. All secondary antibodies were purchased from GE Healthcare.

Quantification of nitrite concentration. The concentration of nitrite in the culture medium was determined by the colorimetric Griess reaction (Grisham et al., 1996), 7 days after changing the media of the astrocytes, using the Griess detection kit for nitrite determination (Molecular Probes-Invitrogen). The assays were performed in triplicates and the experiment was repeated 3 times.

Data Analysis. Statistical analysis was performed using student's t-test and is reported as mean±S.D. Significant t-test values were p<0.05 (*) and p<0.01 (**).

### Example 1.3

### HESC Generate Functional Motor Neurons In Vitro

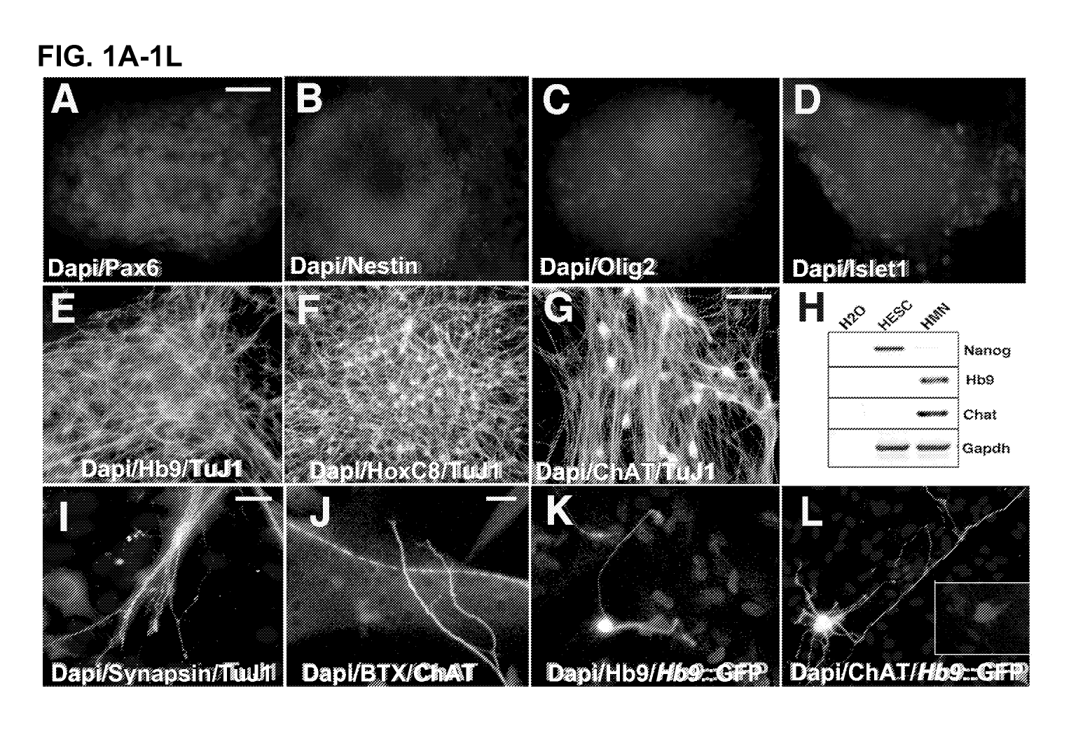

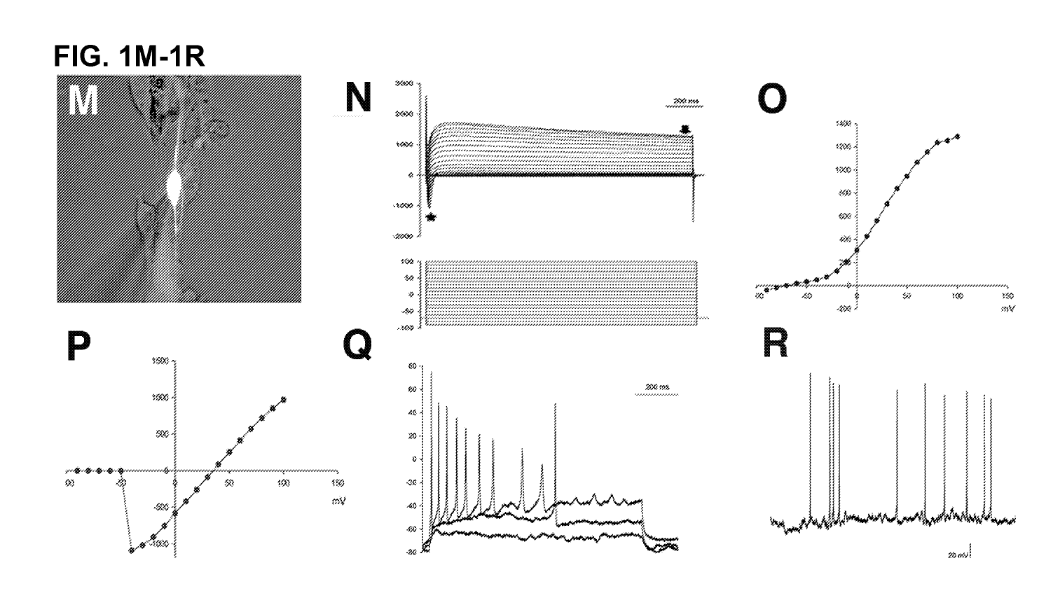

HESC-derived rosettes expressed motor neuron progenitor markers such as Pax6, Nestin, Olig2 and Islet1 after 2-3 weeks of differentiation (FIG. 1A-D). After 4 weeks under differentiation conditions, the cells started to express pan-neuronal markers such as TuJ1 and, after 6-8 weeks, the cells exhibited motor neuron postmitotic lineage-specific markers, such as homeobox gene Hb9, HoxC8 and choline acetyltransferase neurotransmitter, ChAT (FIG. 1E-G). Motor neuron identity was also confirmed at the transcription level by RT-PCR. Accordingly, we detected down regulation of the HESC undifferentiated marker, Nanog, and upregulation of the postmitotic motor neuron markers, Hb9 and ChAT (FIG. 1H). At the 8-week differentiation stage, cells were also positive for synapsin and could incorporate α-bungarotoxin when co-cultured with C2C12 myoblasts, indicating that the cells could form functional neuro-muscular junctions (FIG. 1I,J). Live postmitotic human motor neurons could be visualized after transduction with a lentivirus expressing the green fluorescent protein gene (GFP) under the control of the Hb9 promoter (Lee et al., 2004) (Lenti Hb9::GFP). We confirmed the promoter specificity by co-staining the Hb9::GFP-positive cells with the endogenous Hb9 protein in HESC-derived neurons as well as in rat purified spinal cord motor neurons (FIG. 1K and Figure S2A-C). We performed RT-PCR for the endogenous human Hb9 transcript in sorted Hb9::GFP positive versus Hb9::GFP negative cells and only detected endogenous Hb9 expression in Hb9::GFP positive cells (Figure S2D). The Hb9::GFP positive neurons also co-localized with ChAT marker (FIG. 1L). The functional maturation of the HESC-derived neurons were determined using electrophysiology. Whole-cell perforated patch recordings were performed from cultured HB9-expressing cells that had differentiated for at least 8 weeks in culture (FIG. 1M-R). HESC were successfully differentiated in electrophysiologically active Hb9-expressing human motor neurons to establish a system for modeling ALS using human cells.

### Example 1.4

### Co-Culture Assays

A population of human neurons were consistently generated in vitro that expressed post-mitotic motor neuron markers, made neuro-muscular junctions, and fired action potentials. Subsequently, the human embryonic stem cell (HESC)-derived motor neurons were co-cultured with human primary astrocytes expressing either the wild type or the mutated form of SOD1 protein (SOD1^{WT} or SOD1^{G37R}, respectively).

In the co-cultures, a specific decrease in the number of motor neuron markers were detected in the presence of SOD1-mutated astrocytes, with no detectable effect on other subtypes of neurons. Furthermore, the toxicity conferred by the SOD1-mutated astrocytes was shown to be generated in part by an increase in astrocyte activation and production of ROS. The physiological changes observed in SOD1^{G37R} human astrocytes were well correlated with intensification of the pro-inflammatory activity of the induced nitric oxide enzyme (iNOS or NOS2A), neurosecretory protein chromogranin A (CHGA), secretory cofactor cystatin C(CC or CST3) and NADPH oxidase (NOX2/gp91^{phox} or CYBB) overexpression. Activation of NOX2 and production of oxygen radicals had already been demonstrated to be mediators of microglial toxicity in familial ALS mouse models (Barbeito et al., 2004; Wu et al., 2006).

### Example 1.5

### Expression of Mutated SOD1^{G37R} Protein in Astrocytes Affects Motor Neuron Survival

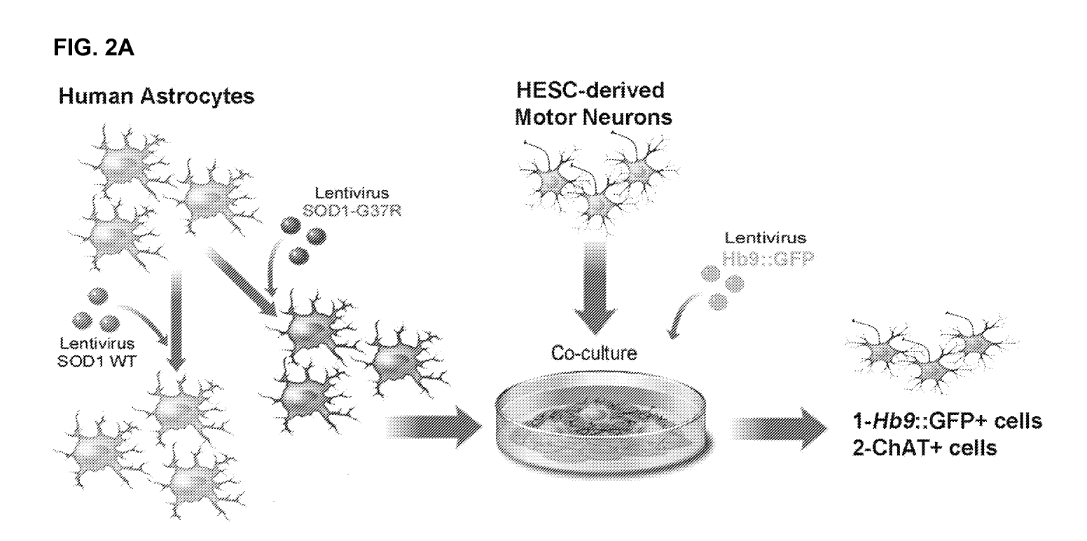

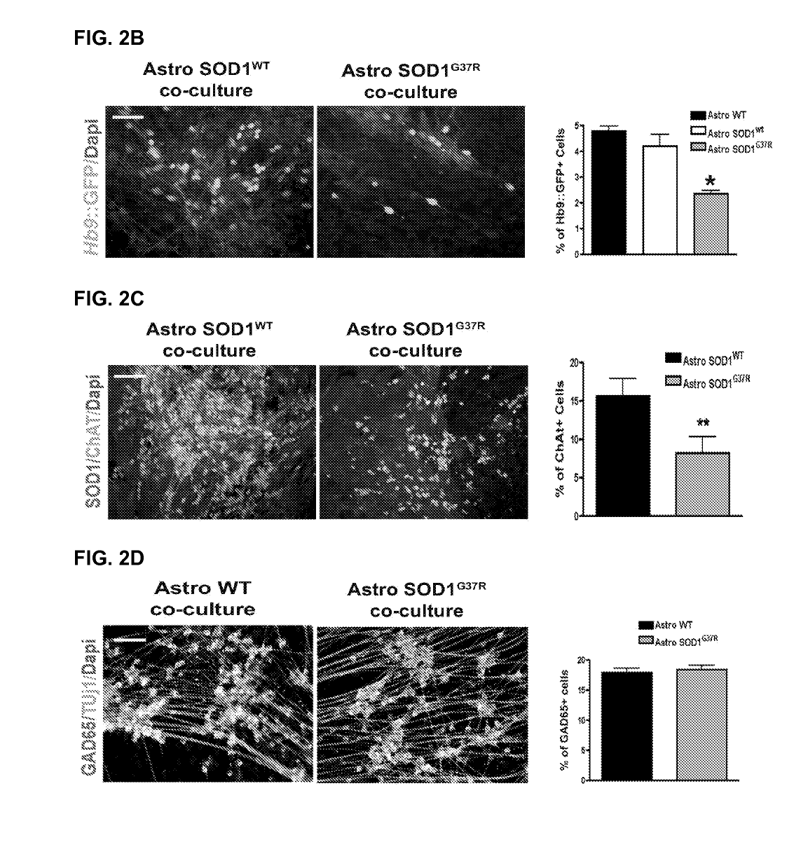

The effects of astrocytes expressing either a wild type (SOD1^{WT}) or mutated (SOD1^{G37R}) form of the human SOD1 protein were examined on the survival of HESC-derived motor neurons upon co-culture. Primary human astrocytes were transduced with a lentivirus vector expressing either SOD1^{WT} or SOD1^{G37R} (Figure S1A,B). The Hb9::GFP motor neurons were co-cultured with SOD^{WT-} or SOD1^{G37R}-expressing astrocytes (FIG. 2A). After co-culture for 4 weeks, cells were subjected to fluorescent activated cell sorting (FACS) for Hb9::GFP quantification (FIG. 2B). A decrease of 49% of Hb9::GFP-positive cells was detected when co-cultured with SOD1^{G37R} astrocytes. For comparison, non-infected human astrocytes were included and did not detect significant differences in the number of Hb9::GFP-positive cells when compared to SOD1^{WT} co-cultures (see graph in FIG. 2B). To further confirm these findings, the number of cholinergic neurons in co-cultures with SOD^{WT} or SOD1^{G37R} astrocytes (FIG. 2C) were counted. A similar decrease (52%) in ChAT-positive cells was detected when co-cultured with SOD1^{G37R} astrocytes. Moreover, the toxic or detrimental effect was specific to the motor neuron population, since other subtypes of neurons concomitantly present in the differentiated cultures, such as GABAergic neurons, were not affected (FIG. 2D). The toxic effect of mutated astrocytes was determined to be specific for glial cell type and was not present in human primary fibroblasts overexpressing SOD1^{WT} or SOD1^{G37R} that were co-cultured with HESC-derived motor neurons (FIG. 93 A-C).

This model consists of co-culturing healthy human motor neurons with human astrocytes carrying either the wild type or mutated SOD1 cDNA. These experiments confirm the role of astrocytes in ALS disease, as motor neuron numbers decreased about 50% in the presence of mutant SOD1-expressing astrocytes. Moreover, the toxicity seemed to be restricted to the motor neuron subpopulation, with no effects on other neuronal subtypes.

### Example 1.6

### Astrocytes Activate an Inflammatory Response in the Presence of SOD1^{G37R}

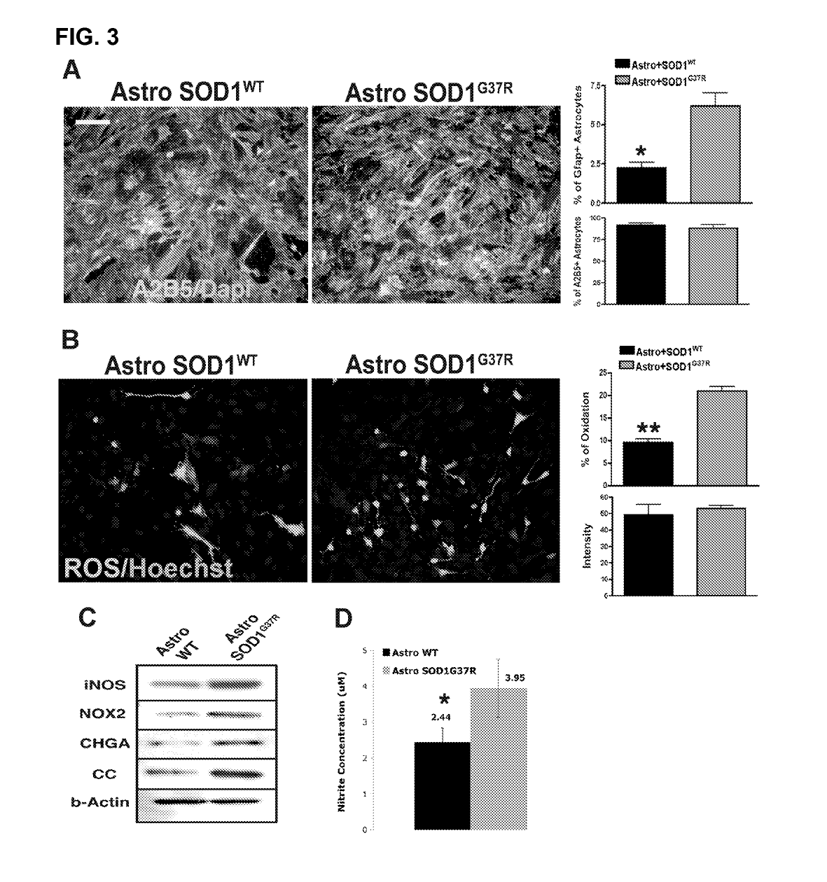

The possible causes of the astrocytic toxicity conferred by the mutated SOD1 to HESC-derived motor neurons was investigated by analyzing the behavior of the mutated astrocytes in culture. Primary astrocytes usually respond to inflammation by activation. Activated astrocytes increase the assembly of their intermediate filaments (produced by glial fibrillary acidic protein; GFAP) and the number and size of the processes extended from the cell body. An=increase of more than 2.5 times the number of activated (GFAP-positive) astrocytes was detected when SOD1^{G37R} was present in comparison to control astrocytes (FIG. 3A). The population of astrocytes was still homogeneous after SOD1 overexpression by staining the cells with A2B5, a general astrocyte marker (FIG. 3A). Moreover, a cell death analysis for both SOD1^{G37R} and SOD1′ astrocytes had similar amounts of propidium iodide (PI) staining (FIG. 71C), thereby confirming that the viability of astrocyte SOD^{G37R} is similar to SOD^{WT}. In parallel, an increase in the number of cells producing ROS by the astrocytes expressing the mutated SOD1 (FIG. 3B) was exhibited, a hallmark of ALS pathology (Barber et al., 2006). The intensity of fluorescence present in the oxidation experiments was calculated but did not detect significant changes between groups (FIG. 3B).

In addition, an increase in the expression of pro-inflammatory factors such as iNOS was observed, an overexpression of the neurosecretory protein known to interact specifically with mutated SOD1, chromogranin A (Urushitani et al., 2006), induction of a superoxide producer enzyme NOX2 (gp91^{phox} subunit) and an increase of cysteine protease inhibitor Cystatin C expression (FIG. 3C). The increment in iNOS enzyme was accompanied by a rise in the NO levels in the SOD1^{G37R} astrocytes conditioned media, indirectly measured by nitrite concentration (FIG. 3D).

The mechanism of astrocyte-specific motor neuron toxicity involves both secretory and inflammatory pathways. Cystatin C (CC), a secretory cofactor involved with inhibition of cysteine proteinases and neurogenesis, has been identified in cerebral spinal fluid (CSF) proteomic profiles as a potential biomarker for ALS (Pasinetti et al., 2006; Taupin et al., 2000). CC is one of the two proteins that immunostain the so-called Bunina bodies, small intraneuronal inclusions that are the only specific pathological ALS hallmark (Okamoto et al., 1993).

These findings suggest that the secretion of mutant SOD1 represents one of the neurotoxic pathways for the non-cell-autonomous nature of ALS.

### Example 1.7

### Astrocyte ROS Production is Reversed by Anti-Oxidants: a Model for Drug Screening

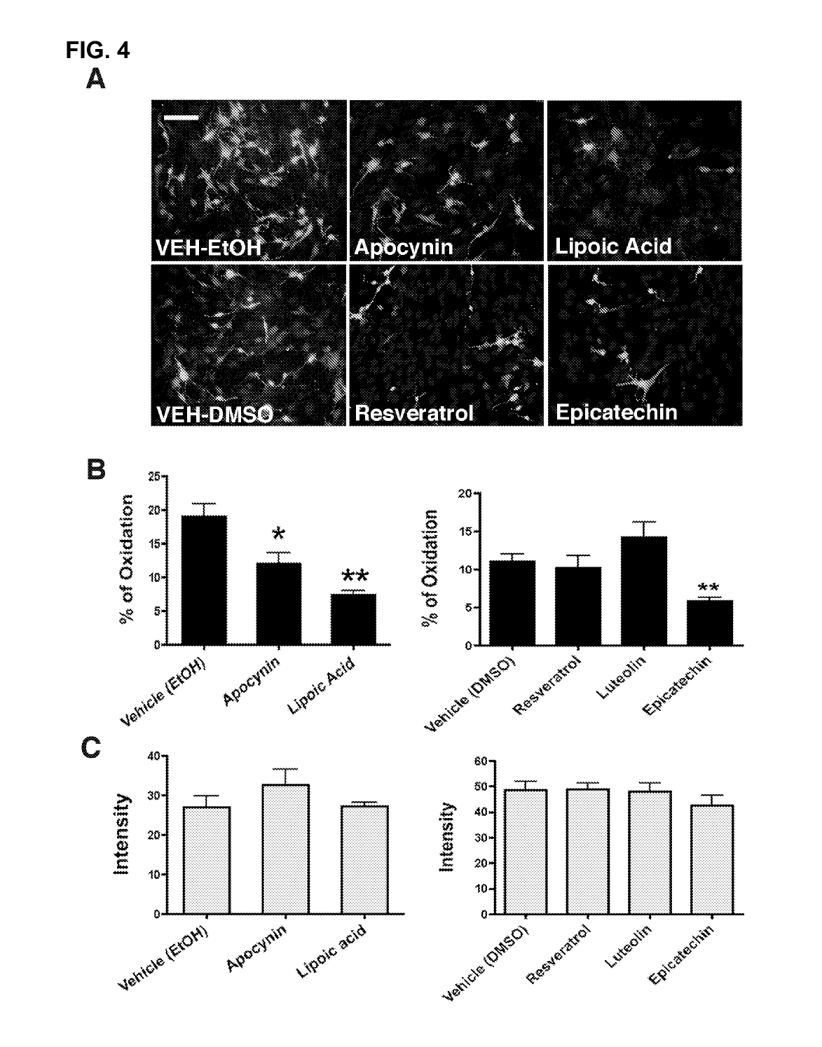

A total of five compounds and their respective vehicles (ethanol (EtOH) or DMSO) were tested in SOD^{G37R} mutated astrocyte cultures to address their anti-oxidant potential (FIG. 4A). Treatment with both NOX2 inhibitor apocynin and anti-oxidant alpha-lipoic acid for 48 hours decreased the percentage of cells that were able to produce ROS (percentage of oxidation) in comparison to treatment with vehicle only (EtOH) (FIG. 4B). Likewise, treatment with the anti-oxidant flavonoid epicatechin decreased the oxidation levels of SOD1^{G37R} astrocytes when compared to vehicle (DMSO). The drugs resveratrol and luteolin, on the other hand, did not seem to have a detectable effect on the number of SOD1^{G37R} astrocytes that are producing ROS.

The compound apocynin was chosen for further verification in a co-culture assay using HESC-derived motor neurons and either SDO1^{WT} or SOD^{G37R} astrocytes. Apocynin treatment rescued the motor neuron survival in the presence of SOD1^{G37R} (FIG. 5), confirming previous observation in SOD1-mutated transgenic mice treated with the same drug (Harraz et al., 2008; Marden et al., 2007; Wu et al., 2006).

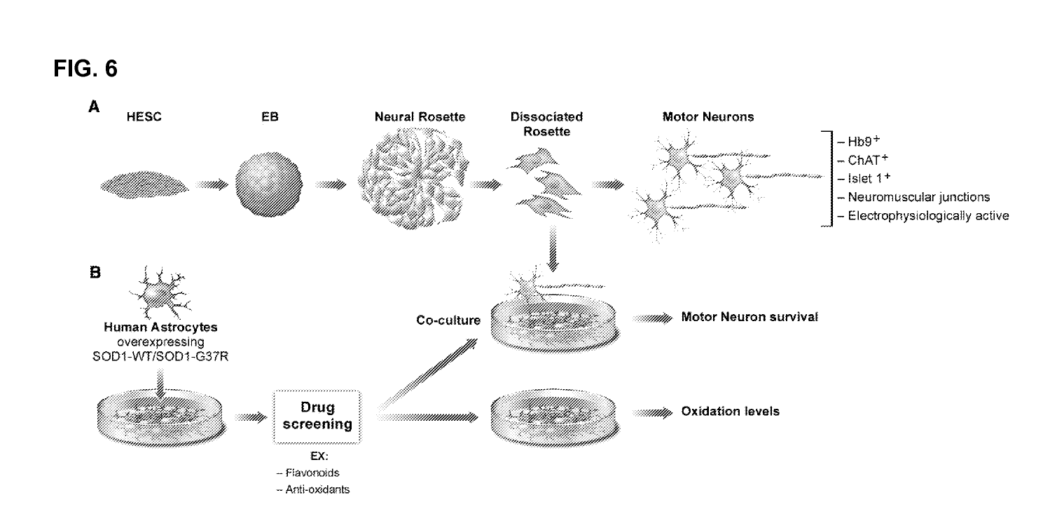

This data shows that anti-oxidant apocynin decreased the ROS production in SOD1-mutant expressing astrocytes, likely by inhibition of NADPH oxidase (NOX2), and in turn restored motor neuron survival. Thus, SOD1-mutant astrocytes may be used as a rapid drug screening test for oxidative damage to identify the best candidates for a following long-term co-culture experiment (FIG. 6).

### Example 2

### A Nurr1/CoREST Transrepression Pathway Attenuates Neurotoxic Inflammation in Activated Microglia and Astrocytes

### Example 2.1

### Experimental Procedures

Mice and isolation of primary cells. C57BL/6 mice were purchased from Charles River and housed according to UCSD protocol. Mouse primary microglia cells and astrocytes from P0 pups were isolated from the standard mixed cortical culture method. After 10-14 days of the culture, microglia cells were isolated from astrocytes by the magnetic sorting using anti-mouse CD11b beads (Miltenyi). Purity of each population was over 98%, as determined by FACS. For the stereotaxic injections, C57BL/6 mice were purchased from Harlan and housed at The Salk Institute following the institutional protocol.

Cell Culture. Primary mouse microglia, mouse astrocyte, Neuro2A (mouse neuroblastoma), 293T, NIH3T3 and Hela cells were cultured in DMEM (Cellgro) supplemented with 10% fetal bovine serum (FBS) and penicillin/streptomycin (Invitrogen). Murine microglial cell line BV2 cells (kindly provided by Katerina Akassoglou) and macrophage RAW264.7 were maintained with DMEM supplemented with 10% FBS (low endotoxin, Hyclone) and penicillin/streptomycin. SK—N—SH (human neuroblastoma) cells were maintained in aMEM supplemented with 10% FBS and antibiotics. PC12 (rat pheochromocytoma) cells were cultured with 10% horse serum (Hyclone), 5% FBS and antibiotics. SK—N—SH and PC12 cells were differentiated following ATCC protocol. Mouse neuronal stem cells (NSCs) from ventral mesencephalon were cultured and differentiated following the manufacturer's protocol (StemCell Technologies). Primary human microglia cells were purchased from Clonexpress and primary human astrocytes were obtained from ScienCell and maintained following the manufacturer's protocol.

Luciferase assay. The RAW264.7 mouse macrophage cell line was transiently transfected with iNOS- or TNFα-promoters directing luciferase expression, as previously described (Ghisletti et al., 2007; Pascual et al., 2005). For siRNA experiments, RAW264.7 cells were co-transfected with siRNAs (40 nM) using Transmessenger reagent (Qiagen) for 48 h before activation with LPS. In all transfections, cells were stimulated with 0.1 μg/ml LPS (Sigma) and assayed for luciferase activity 6 h later for TNFα and 8 h later for iNOS. Transfection experiments evaluated each experimental condition in triplicate and results are shown as fold induction compare to unstimulated samples and LPS-stimulated samples and standard deviation. In all promoter assays, fold induction represents LPS-stimulated promoter activity divided by promoter activity in unstimulated cells. Error bars represent standard deviations (SD).

Chromatin immunoprecipitation (ChIP) assays. ChIP assays were performed as previously described (Ghisletti et al., 2007; Pascual et al., 2005).

RNA isolation and quantitative PCR. Total RNA was isolated by RNAeasy kit (Qiagen) from cells or SN samples microdissected from the brain. One microgram of total RNA was used for cDNA synthesis using Superscript III (Invitrogen), and quantitative PCR was performed with SYBR-GreenER (Invitrogen) detected by 7300 Real Time PCR System (ABI). The sequences of qPCR primers used for mRNA quantification in this study were obtained from PrimerBank (Wang and Seed, 2003).

Statistical analyses. Standard deviation, Chi-square and two-tail Student's t-test were performed with the Prism 4 program. p<0.01 was considered significant. For IHC and IF analyses, Bonferroni was used for post hoc analysis when a significant difference was found with ANOVA. Unpaired two-tailed t test was used for other comparisons, including comparisons between control and injected sides within one group. All data are presented as mean±SD.

Stereotaxic injection of lentivirus and LPS in the mouse SN in vivo. Preparation of lentivirus is described in the section of plasmids and reagents. Groups were defined by lentiviral type (shCtrl, shNurr1-1 and shNurr1-2). Mice were anesthetized using a mixture of ketamine/xylazine (100 mg/kg, 10 mg/kg) and immobilized in a stereotaxic apparatus. The stereotaxic injection site into the right SN was AP −3.3 mm, ML −1.2 mm, DV −4.6 mm from bregma (Franklin and Paxinos, 2008). A stainless steel cannula (5 μl Hamilton syringe) was inserted and one deposit of 1.5 μl of lentivirus was slowly injected over a 2-minute period. Five minutes passed before the needle was removed to minimize retrograde flow along the needle track. Two days after the lentivirus injection, a single 1-1 μl injection of 5 μg of LPS (Sigma) or 1 μl of PBS was delivered over a 2-minute period into the same coordinates. An additional group received LPS/PBS control injections without preceding lentiviral injections. For A30P a-Synuclein injection, the same technique was applied to the paradigm described in FIG. 28B top panel.

Microdissection of SN. Mice (n=4 per group) were euthanized 6 h after the LPS injection. The brains were removed, the injected SN was dissected under a dissection microscope and the tissue was processed for qPCR.

Immunohistochemistry (IHC) and immunofluorescence (IF). Experimental animals were anesthetized and perfused transcardially with 0.9% saline followed by 4% paraformaldehyde. The brain samples were postfixed with 4% paraformaldehyde overnight and equilibrated in 30% sucrose. Coronal sections of 40 μm an were prepared with a sliding microtome and stored in cryoprotectant (ethylene glycol, glycerol, 0.1 M phosphate buffer pH 7.4, 1:1:2 by volume) at −20° C. IHC and colabeling IF for free-floating sections were performed with the following primary antibodies: rabbit anti-Ibal (1:500, Wako Chemicals), mouse anti-tyrosine hydroxylase (1:250, Chemicon), guinea pig anti GFAP (1:1000, Advanced Immuno) and rabbit anti Caspase 3 (1:500, Cell Signaling). For IHC, sections were stained with donkey anti-mouse biotinylated, antibody (1:500, Jackson Immuno Research), followed by the avidin-biotin-peroxidase complex 1:100 (Vectastain Elite). The peroxidase activity of immune complexes was revealed with a solution of TBS containing 0.25 mg/ml 3,3′-diaminobenzidine (Vector Laboratories, Burlingame, USA), 0.01% H2O2, and 0.04% NiCl2. For IF, tRHOX-tagged donkey anti-mouse and FITC-tagged donkey anti-rabbit antibodies were used. 4,6-Diamidino-2-phenylindole (DAPI, 1:1000, Roche) was used to reveal nuclei.

Stereology and confocal microscopy. To determine cell numbers of TH-immunoreactive neurons in the SN, an unbiased stereological method according to the optical fractionator principle was used (Gundersen et al., 1988). Every fourth section (120 μm interval) was selected from each animal and processed for immunostainings for TH. The reference volume was determined by tracing the areas using a semi-automatic stereology system (Stereoinvestigator, MicroBrightField). No counting frames were used here, but these regions were exhaustively counted on each section. In the lentivirus-treated groups, a number of TH-positive cells had pathological morphology, indicating a degenerative process on the side of the injection, as shown in FIG. 10B and FIG. 18E. We made use of a morphological distinction between “normal” and “pathological” TH-positive cells. The first category (“normal”) was defined as TH-positive cells that displayed long dendritic processes or with a cytoplasm surrounding the nucleus that measured at least the same amplitude as the nucleus itself at its smallest area. TH-positive cells were determined as “pathological” when they had no processes, were smaller than 2-fold the nucleus size, or had abnormal surfaces. According to this distinction, we classified cell numbers and morphologies of TH-positive cells.

For analyzing the interplay between Iba-1 and TH+ cells, a confocal laser microscope (Nikon) equipped with a 40×PL APO oil objective was used. All animals were coded in this study, and a blinded analysis was used for quantitative comparisons.

Conditioned media (CM) assays. Primary mouse microglia and astrocytes were infected with lentivirus directing expression of non-targeting control or Nurr1-specific shRNAs. BV2 cells were infected with shCtrl- or shNurr1-lentivirus or transfected with siRNAs against Nurr1 or components of the CoREST complex. Cells were then treated with 0.1 μg/ml LPS for 24 h. For primary mouse microglia and astrocytes, cells were infected with lentivirus carrying shRNA against Nurr1 or control. Cells were stimulated with 0.1 μg/ml LPS for 2 h and washed with PBS extensively to avoid carry over of LPS to the next step. CMs were filtered through 0.45 μm filters and frozen at −80° C. Target cells were cultured with CMs for 24 h and TUNEL assays were performed with Cell Death Detection ELISAplus kit (Roche) following the manufacturer's protocol. For in vitro differentiated cells from mouse NSC, In Situ Cell death detection kit (Roche) was used for TUNEL assay. NSC-derived cells were stained with anti-Tyrosine hydroxylase (TH-16, Sigma), anti-GABA (GB-69, Sigma) and anti-GFAP (131-17719, Molecular Probes) and visualized by goat-anti mouse IgG-Alexa-488 (Molecular Probes). Nuclei were visualized by DAPI (Invitrogen) staining.

Plasmids and lentivirus production. Flag-tagged full-length (FL) mouse Nurr1 was cloned into p3XFLAG-CMV-7.1 vector (Sigma). Mutant constructs of Nurr1 were generated with the Quick-change site-direct mutagenesis kit (Stratagene). HA-tagged mouse CoREST-FL was cloned into pcDNA3 expression vector (Invitrogen). DBD from Nurr1 was cloned into pCMV-Myc vector (Invitrogen). Various mutants with non targeting Nurr1 used for the reconstitution of shNurr1-BV2 cells were cloned into pHAGE lentivirus vector kindly provided from Jeng-Shin Lee and Richard Mulligan. All smart-pool siRNAs were purchased from Dharmacon. Retrovirus pSM2c carrying two independent shRNAs directed against mouse Nurr1 (shNurr1-1 and shNurr1-2) were purchased from Openbiosystems. Retrovirus production and infection into BV2 cells were performed according to the manufacturer's protocol. Fragments containing U6 promoter, miR30 and shRNA were isolated from pSM2c and subcloned into the lentivirus vector p156RRLsinPPTCMV-GFP-PREU3Nhe (kindly provided by Inder Verma). Lentivirus encoding A30P mutant of α-Synuclein was kindly provided by Roberto Jappelli and Roland Riek. Lentivirus packaging was done using Virapower (Invitrogen) and 293T cells as a packaging cell line according to the manufacturer's protocol. pGIPZ-lentivirus carrying shRNAs were purchased from Openbiosystems and virus production was performed following the manufacturer's protocol. Validation of siRNA or shRNA used in this study was performed by either qPCR or Western blotting shown in FIG. 27.

ChIP assay. For each experimental condition, 2×107 BV2 cells or 6×106 mouse primary astrocytes were used. Cells were stimulated with 1 μg/ml LPS for BV2 cells and 10 ng/ml IL1β for astrocytes for the indicated time before crosslinking for 10 minutes with 1% formaldehyde. For in vivo ChIP, single cell suspensions were made from microdissected SN samples using cell strainers (BD Falcon) in prior to the crosslinking Anti-Nurr1 (E-20, Santa Cruz Biotechnology) anti-p65 (C-20, Santa Cruz Biotechnology), anti-CoREST (Millipore) or control rabbit IgG (Santa Cruz Biotechnology) were used for IP. A 150-bp region of the iNOS promoter was amplified spanning the most proximal NF-κB site to the start of transcription as described before (Ghisletti et al., 2007; Pascual et al., 2005). A 150-bp region of the mouse proximal TNFα promoter was amplified spanning the NF-κB site. Quantitative PCR (qPCR) was performed with SYBR-GREEN PCR master Mix (ABI) or SYBR-GreenER (Invitrogen) and analyzed on a 7200 real time PCR system (ABI).

SUMOylation assays. In vivo SUMOylation experiments were performed as described before (Ghisletti et al., 2007; Pascual et al., 2005). Briefly, whole cell extract was prepared in the presence of N-Ethylmaleimide (Calbiochem) from HeLa and NIH3T3 cells transfected with Flag-tagged wild type Nurr1 or lysine mutants and SUMO-1, SUMO-2 or SUMO-3, Ubc9 and PIAS4 expression vectors or the indicated siRNAs. Extracts were resolved by SDS-PAGE and immunoblotted using anti-Flag antibody (Sigma).

Co-immunoprecipitations and Western blotting. Hela cells or NIH3T3 cells were transfected using Lipofectamine 2000 reagent (Invitrogen) following manufacturer's protocol. Transfected cells were stimulated by 10 ng/ml recombinant human or mouse mIL1β (R & D system) for the indicated times prior to harvesting. BV2 cells were treated with 1 μg/ml LPS and mouse primary astrocytes were stimulated with 10 ng/ml IL1β for the indicated times. Cells were lysed with hypotonic buffer (10 mM HEPES pH7.4, 320 mM Sucrose, 5 mM MgCl2, 1% Triton X-100) supplemented with proteinase inhibitor cocktail (Sigma), 2 mM Na3VO4 (Sigma) and 50 nM Calyculin A (Calbiochem) using a Dounce homogenizer. After centrifugation, nuclei were washed twice with hypotonic buffer without Triton X-100. Then cells were resuspended in hypertonic buffer (50 mM Tris pH8.0, 500 mM NaCl, 1 mM EDTA, 10% Glycerol) with proteinase and phosphatase inhibitors as described before and sonicated briefly. For endogenous co-IP experiments, anti-Nurr1 (E-20, Santa Cruz), anti-CoREST (E-15, Santa Cruz and Millipore) and anti-p65 (C-20, Santa Cruz) were used for IP and Western blotting. For immunoprecipitation of tagged protein, M2 anti-Flag-agarose (Sigma) and HA-agarose (Covance) beads were used for Flag and HA tagged proteins, respectively. For the loading control of whole cell lysate samples, anti-actin antibody (Oncogene) was used.

GST-pull down. Wild-type full-length mouse Nurr1 or CoREST were cloned in pGEX-6P vector (GE healthcare). pGEX-6P vectors were transformed into BL21 or ArcticExpress E. Coli (Stratagene) and GST-fusion proteins were purified by Glutathione Sepharose 4 Fast Flow (GE healthcare) following the manufacturer's protocol. 35S-labeled CoREST and Nurr1 were generated using TNT-T7 in vitro transcription/translation kit (Promega). GST-pull down assays were performed as described before (Ogawa et al., 2005). In the case of GST-Nurr1 and TNT-p65, the GSK3β kinase reaction was performed prior to the binding reaction using purified GSK3β following the manufacturer's protocol (Millipore).

### Example 2.2

### Results

Nurr1 protects TH+ neurons from LPS-induced inflammation in vivo. Analysis of Nurr1 protein and mRNA levels in primary human and mouse microglia, primary human astrocytes, and the BV2 microglia cell line demonstrated significant protein expression under basal conditions and induction of Nurr1 mRNA in microglia in response to LPS (FIGS. 17A-E and data not shown). Similarly, Nurr1 mRNA was detected in the SN of the mouse brain under basal conditions and was induced approximately 2-fold by 6 h following stereotaxic injection of LPS (FIG. 17F). Nuclear Nurr1 protein colocalized with the microglia marker F4/80 in the SN (FIG. 18E). To investigate the potential role of Nurr1 in PD pathology in the mouse brain, we evaluated the impact of reducing Nurr1 expression. Since Nurr1-deficient mice die shortly after birth, we performed stereotaxic injections of lentiviruses encoding two independent shRNAs against Nurr1 (shNurr1-1 and shNurr1-2) or control shRNA (shCtrl) into the SN of adult wild-type mice (FIG. 18A). shNurr1-1 and -2 efficiently and specifically reduced Nurr1 mRNA expression in the SN, as determined by qPCR as well by immunostaining (Sup. FIGS. 18B-E). A comparison of lentivirus-directed GFP expression with cell-specific markers indicated preferential transduction of non-neuronal cells, including microglia and astrocytes (FIG. 18G). The lentiviral injection was followed two days later by injection of LPS into the same coordinates. We then analyzed the magnitude of the inflammatory response by qPCR 6 h after the LPS injection and quantified tyrosine hydroxylase (TH)+ neurons by immunohistochemistry (IHC) 7 days after LPS injection.

Loss of TH+ neurons following LPS injection normally takes 2-3 weeks (Meredith et al., 2008). However, stereological analysis revealed a significant decrease in TH+ neurons in the SN of shNurr1 lentivirus-injected mice compared to shCtrl-injected animals after only 7 days of LPS treatment (FIGS. 10A and B). Interestingly, a pathological morphology of TH+ neurons with reduced or absent processes and alterations in the size and shape of the cells was observed more often in the shNurr1 groups (FIG. 18E and FIG. 10B). In addition, the pathological TH+ cells were observed close to activated microglia cells (FIG. 18F). The accelerated loss of TH neurons following Nurr1 knockdown required LPS injection, as it was not observed in buffer (PBS)-injected animals (FIGS. 10C and D). This result excludes the possibility that loss of TH+ staining was simply due to loss of Nurr1 expression in neurons. In addition, LPS injection was associated with detection of caspase-3 cleavage, suggesting that loss of TH+ cells was due to cell death rather than to loss of TH expression (FIG. 18G) (Sakurada et al., 1999). Reduction of Nurr1 expression in the SN also resulted in exaggerated expression of inflammatory mediators in response to LPS injection, including IL1β, TNFα and iNOS (FIGS. 10E-G). In concert, these experiments indicate that Nurr1 limits inflammatory responses in the CNS and protects TH+ neurons from LPS-induced toxicity.

Based on recent findings that transgenic expression of wild-type or mutant forms of α-Synuclein potentiates LPS-mediated loss of TH+ neurons (Gao et al., 2008), we examined whether Nurr1 exerted neuro-protective effects in the situation of overexpression of an α-Synuclein mutant (A30P) associated with familial PD. We again employed stereotaxic injection of shNurr1- or shCtrl-lentivirus in combination with lentivirus encoding mutant α-Synuclein (A30P). A30P expression alone caused weak inflammation in the SN, whereas reduction of Nurr1 expression in the context of A30P expression resulted in a dramatic increase in expression of numerous inflammatory response genes, including TNF and IL1β, and significant loss of TH+ neurons (FIGS. 19A-C and data not shown).

Glia-mediated inflammation contributes to the death of TH+ neurons. To define the cell types responsible for LPS-mediated inflammation in the SN, we evaluated the responses of human and mouse microglia, astrocytes and neurons to LPS. These experiments demonstrated that microglia are orders of magnitude more responsive than astrocytes or neurons, exemplified by the pattern of TNFα induction in primary mouse microglia and astrocytes and the neuronal Neuro 2A (mouse neuroblastoma) cell line (FIG. 11A) as well as in corresponding human cells (FIG. 20F). These results are consistent with the expression patterns of TLR4, co-receptors and down-stream signaling molecules in neurons and glial cells (FIGS. 20A-E). Although, TLR4 expression was virtually absent from the neuronal cell lines examined, we tested whether LPS could directly induce the death of these cells. Three different neuronal cell lines, Neuro2A, SK—N—SH and PC12, were incubated with LPS for 24 h but no significant cell death was observed by TUNEL assay or caspase-3 cleavage, in contrast to the effects of TNFα plus cyclohexamide (CHX) treatment (FIG. 11B and FIG. 20G). In addition, knockdown of Nurr1 in Neuro2A cells did not increase the sensitivity to LPS or death signaling (TNFα plus CHX) as determined by TUNEL assay (FIG. 11C).

Based on these results, we evaluated the consequences of reducing Nurr1 expression in microglia on LPS responses. Knockdown of Nurr1 expression in BV2 microglia using specific lentivirus-encoded shRNAs led to significant increases in LPS-dependent expression of inflammatory mediators, including TNFα iNOS and IL-1β (FIGS. 11D-F and data not shown). Similar results were observed in the primary mouse (FIG. 21 and FIGS. 22 A-C) and human (data not shown) microglia. To explore whether loss of Nurr1 in microglia resulted in secretion of mediators exhibiting preferential toxicity for TH+ neurons, we knocked down Nurr1 expression using lentivirus-encoded shRNAs in BV2 cells and tested the activity of conditioned media (CM) after LPS stimulation on in vitro differentiated neurons and glial cells derived from mouse neuronal stem cells (NSC). As shown in FIG. 11G-I, the CM from shNurr1-BV2 cells resulted in the death of nearly all TH+ neurons, with a significantly smaller effect on gamma-aminobutyric acid (GABA)-positive neurons and no significant consequence on glial fibrillary acidic protein (GFAP)-positive astroglial cells.

Experiments using neuron and glia co-culture in vitro suggest that activation of innate immunity in the CNS can trigger neuronal death (Lehnardt et al., 2003). Since NSC-derived neurons always co-exist with astrocytes, it is possible that astrocytes contributed to the neurotoxic effect of the microglia CM. To explore this possibility, we performed sequential CM experiments employing isolated primary microglia and astrocytes and using Neuro2A cells as a read-out for neurotoxicity. Primary murine astrocytes and microglia were infected with shCtrl- and shNurr1-lentivirus as used for the injection into the SN. Cells were then stimulated with LPS and CM was harvested as described in FIG. 11G. CM of microglia infected with shNurr1 induced significant cell death in Neuro2A cultures, whereas CM of astrocytes infected with shNurr1 had much less effect on the death of Neuro2A cells. Intriguingly, sequential conditioning of media from microglia to astrocytes or astrocytes to microglia indicated that astrocytes significantly amplified the production of neurotoxic factors when exposed to microglia-conditioned media (FIG. 11J lane 2 to lane 6 and 7). This effect was further increased when expression of Nurr1 was reduced in astrocytes (FIG. 11J lane 3 to lane 8 and 9). Based on these data, we conclude that microglia are the initial responders to LPS-mediated inflammation and that astrocytes amplify the production of neurotoxic factors after the microglial activation. The knockdown of Nurr1 in both microglia and astrocytes increases the toxicity of CM, suggesting that Nurr1 plays an essential role in inhibiting the production of neurotoxic factors in both cell types.

Nurr1-mediated transrepression requires GSK3n-dependent recruitment of Nurr1 monomers to p65. Chromatin immunoprecipitation experiments indicated that Nurr1 was recruited to LPS-responsive promoters following LPS treatment, exemplified by the TNFα promoter (FIG. 12A), suggesting that it was acting locally to repress transcription. Two different general mechanisms of NR-mediated repression have been described: active repression, involving sequence-specific DNA binding, and transrepression, involving tethering of NRs to negatively regulated target genes via protein-protein interactions (Glass and Ogawa, 2006). A mutant of Nurr1 (Nurr1C280A/E281A, CEAA), defective for sequence-specific DNA binding and unable to activate NGFI-B responsive element (NBRE)-luciferase, a reporter for Nurr1 monomer-binding, was fully able to repress iNOS induction by LPS (FIG. 12B). In addition, mutations directed at the heterodimerization (1-box) domain (Aarnisalo et al., 2002) of Nurr1 (Nurr1K555A/L556A/L557A, KLL) that prevented its ability to activate a Nurr1/RXR-dependent (DR5)-promoter did not interfere with Nurr1-mediated repression of iNOS (FIG. 12B). On the other hand, this I-box mutation increased transcriptional activation of Nurr1 monomers through the NBRE element, as previously reported (Aarnisalo et al., 2002) (FIG. 22D). SUMOylation of NRs has recently been established to play important roles in transrepression (Ghisletti et al., 2007; Pascual et al., 2005). Since it is known that Nurr1 interacts with the protein inhibitor of activated STAT (PIAS) 4 (Galleguillos et al., 2004), which is a SUMO E3 ligase, we examined whether SUMOylation is also involved in Nurr1-mediated repression. As shown in FIG. 12C, knockdown of Ubc9, an essential E2 enzyme for SUMOylation (Hay, 2005), reversed Nurr1-mediated repression of iNOS, suggesting that SUMOylation is required. Next, we confirmed that Nurr1 could be SUMOylated with SUMO2 and SUMO3 using PIAS4 as an E3 ligase (FIGS. 22E and F). Interestingly, SUMOylation of Nurr1 could be signaling-dependent, since IL1β stimulation could induce SUMOylation of Nurr1 in the absence of overexpression of PIAS4 (FIG. 22F). Mutational studies demonstrated that lysine 558 and, to a lesser extent, lysine 576 are essential SUMO sites of Nurr1 (FIG. 12D). Since both K558R and K576R mutants are located in the ligand binding domain and close to the I-box and RXR is not required for repression activity (FIG. 12B and FIG. 22D), we hypothesize that SUMOylation is required for monomerization of Nurr1. The K558R and K576R mutants were less able to activate the NBRE reporter and preferentially activated the DR5 reporter (FIGS. 22G and H), and they altered the Nurr1-mediated repression of iNOS-reporter assay (FIG. 12E), consistent with SUMOylation of Nurr1, specifying a monomer configuration that is a prerequisite for transrepression.

Since transrepression requires the tethering of NRs to other transcription factors, we tested whether Nurr1 could bind to transcription factors involved in inflammation, such as NF-κB. Co-immunoprecipitation (Co-IP) assays of Nurr1 in BV2 cells showed interaction with NF-κB-p65 that was significantly enhanced by LPS treatment and independent of changes in Nurr1 protein levels (FIG. 12F and FIG. 22A). Phosphorylation of Serine-468 (S468) in p65 is associated with negative regulation of NF-κB signaling (Buss et al., 2004) and can be mediated by GSK3β, which is activated following TLR4 stimulation in human monocytes (Martin et al., 2005). Furthermore, inactivation of GSK3β results in increased NF-κB-dependent transcription of TNFα without changing the kinase activity of the IKK complex or the nuclear translocation of p65 (Buss et al., 2004). Therefore, we hypothesized that 5468 phosphorylation of p65 by GSK3β might provide the docking site for tethering of Nurr1. Consistent with this possibility, the GSK3β-specific inhibitor SB216763 (SB21) inhibited the interaction of Nurr1 and p65 in BV2 cells in a dose-dependent manner (FIG. 12G and FIG. 23D). SB21 also prevented the recruitment of Nurr1 to the TNFα-promoter, as determined by Chromatin immunoprecipitation (ChIP) assay (FIG. 12A). To further confirm GSK3β involvement, we performed TNFα-luciferase reporter assays in RAW264.7 cells cotransfected with a kinase-dead mutant of GSK3β (GSK3β-K85R mutant, GSK3β-KD). GSK3β-KD expression abolished the Nurr1-mediated transrepression of the TNFα-promoter in a dose-dependent manner (FIG. 23B). Furthermore, knockdown of GSK3β completely prevented Nurr1-mediated iNOS repression (FIG. 12H). We further validated the contribution of phospho-S468 in p65 by exchanging S468 for alanine (S468A). The p65 S468A mutant, but not wild-type p65, reversed Nurr1-mediated iNOS repression in RAW264.7 cells in a dose-dependent manner (FIG. 12I and FIG. 23E). Finally, GSK3β stimulated the in vitro interaction of Nurr1 with wild-type p65 but not with p65-S486A (FIG. 23C).

The CoREST-repressor complex is required for Nurr1-mediated transcriptional repression. Transcriptional repression requires the recruitment of enzymatically active multiprotein complexes assembled on central scaffolding proteins referred to as co-repressors. Therefore, we sought to identify the co-repressors required for Nurr1-mediated transrepression. We used siRNAs against various candidate corepressors in the iNOS-luciferase reporter assay and identified CoREST as being essential for Nurr1-mediated repression (FIG. 13A and FIG. 24A). CoREST has been considered to be dedicated to repression of neuronal genes in non-neuronal cells or early precursors by binding to neuron-restrictive silencer factor (NRSF)/RE1-silencing transcription factor (REST) (Ballas et al., 2005). CoREST assembles many chromatin-modifying enzymes, including histone methyltransferase G9a, histone demethylase, lysine-specific demethylase (LSD1) and histone deacetylase (HDAC) 1 and 2 (Shi et al., 2003). Using Nurr1-mediated repression of iNOS-luciferase with knockdown of various CoREST complex components, we observed that G9a, LSD1 and HDAC1 were also required for Nurr1-CoREST-mediated repression (FIG. 24B). Using co-IP, we also observed a physical association of Nurr1 and CoREST in BV2 cells (FIG. 13B and FIG. 24C) that was strongly enhanced by LPS treatment. Although the CoREST complex consists of many proteins, the interaction between Nurr1 and CoREST seemed to be direct, as indicated by in vitro GST-pull down assay (FIG. 24D). This interaction was mediated by the DNA-binding domain of Nurr1 (Nurr1-DBD) (FIG. 24E). When Nurr1-DBD was overexpressed in Hela cells, the interaction between Nurr1 and CoREST was inhibited in a dose-dependent manner (FIG. 24F). Furthermore, overexpression of the Nurr1-DBD in RAW264.7 cells altered Nurr1-mediated repression of iNOS-promoter activity (FIG. 24G).

Since Nurr1 can be phosphorylated by serine/threonine kinases (Nordzell et al., 2004), we speculated that signal-dependent phosphorylation might contribute to the Nurr1-CoREST interaction. Nemo-like kinase (NLK) received our attention because NLK is known to be involved in the repression of various transcription factors (Yasuda et al., 2004). NLK cooperates with TGFβ-activating kinase 1 (TAK1) and homeodomain-interacting kinase 2 (HIPK2) in Wnt signaling (Kanei-Ishii et al., 2004). Therefore, we first evaluated the consequences of knockdown of TAK1, HIPK2 and NLK in RAW264.7 cells with respect to Nurr1-mediated transrepression. Knockdown of NLK abolished the repression of iNOS-promoter activity, whereas HIPK2 knockdown was much less effective and TAK1 had no effect (FIG. 13C and FIG. 25A). Furthermore, overexpression of kinase-dead NLK (NLKK155M, NLK-KD) in RAW264.7 cells inhibited Nurr1-mediated repression of iNOS in a dose-dependent manner (FIG. 25B). Kinase assays showed that Nurr1, but not CoREST, could be phosphorylated by active NLK in vitro (FIG. 13D). Finally, Nurr1-CoREST interaction was significantly reduced by NLK-knockdown in BV2 cells (FIG. 13E).

To confirm whether CoREST was indeed localized to NF-κB target gene promoters in association with p65 and Nurr1, we performed ChIP assays of the iNOS- and TNFα-promoters in BV2 cells. The occupancy of NF-κB-p65, Nurr1 and CoREST on both the TNFα- and iNOS-promoters by all three proteins was strongly increased upon LPS stimulation (FIG. 13F, FIG. 25C). On the iNOS-promoter, which exhibits relatively slower activation kinetics, p65 binding preceded the binding of Nurr1, which in turn preceded recruitment of CoREST (FIG. 13F). To verify whether this system is indeed functional in vivo, we performed ChIP assay from microdissected SN after the stereotaxic injection of LPS into mouse SN. Consistent with in vitro data, Nurr1 is recruited to the iNOS- and TNFα-promoters after the LPS stimulation in SN (FIG. 13G, FIG. 25D). Finally, we asked whether Nurr1 was indeed essential for the recruitment of the CoREST complex to target gene promoters. ChIP experiments were performed using shNurr1- or shCtrl-BV2 cells. In the absence of Nurr1, CoREST was not recruited to the TNFα-promoter (FIG. 13H, left panel). Interestingly, under these conditions, p65 was present at the TNFα-promoter for extended times (FIG. 13H, right panel). Acetylation of p65 regulated by HDACs including HDAC1 is known to determine the duration of transcription (Ashburner et al., 2001). HDAC1 is recruited to TNFα or iNOS-promoter in a LPS-dependent manner; however, this recruitment is severely impaired in the absence of Nurr1 (FIG. 24E). Finally, to verify an in vivo role for the molecules identified to be involved in Nurr1/CoREST transrepression pathway, BV2 cells were transfected with siRNAs targeting various molecules and were tested for the ability to increase the production of neurotoxic factors. As shown in FIG. 26A, knockdown of each of the molecules engaged in this Nurr1/CoREST-mediated transrepression pathway induced significantly higher death of Neuro2A cells compared to control siRNA, as detected by TUNEL ELISA assay

Nurr1 represses the production of neurotoxic factors in astrocytes. The observation that astrocytes could amplify the neurotoxic effects initiated by microglia (FIG. 11J) suggested that pro-inflammatory cytokines secreted by activated microglia such as TNFα and IL1β could activate the astrocytes and induce the transcription of inflammatory neurotoxic mediators (FIGS. 11D-F and FIGS. 22A-B). Consistent with this possibility, the receptors for TNFα and IL1β are highly expressed in primary mouse and human astrocytes, but not microglia (FIGS. 14A-B, FIGS. 27A-B). When stimulated with IL1β or TNFα in vitro, both human and mouse astrocytes, but not microglia, activated the transcription of iNOS genes (FIG. 14C and FIG. 27C). Next we investigated whether Nurr1 could also act as a transcriptional repressor in astrocytes. Like TLR stimulation in microglia, TNFα and IL1β stimulation increases the mRNA level of Nurr1 in mouse and human primary astrocytes (FIG. 14D and FIG. 27D). However, similar to microglia, Nurr1 protein expression is not dependent on the stimulation, as Nurr1 expression is observed in primary mouse astrocytes without IL1β stimulation (FIGS. 17E and F). These data suggested that Nurr1 also participates in a signal-dependent negative feedback mechanism in astrocytes. To test this possibility, primary mouse and human astrocytes were infected with the shCtrl- and shNurr1-lentivirus used before. Activated astrocytes can up-regulate many pro-inflammatory genes, including the iNOS and Ncf1 genes upon IL1β and TNFα stimulation, which are essential enzymes for NO and reactive oxygen species (ROS) production, respectively. Knockdown of Nurr1 in astrocytes drastically increased mRNA expression of both iNOS and Ncf1 in response to IL1β and TNFα and up-regulated NO production (FIGS. 14E-G and FIGS. 27E-F). Furthermore, activated astrocytes can produce macrophage colony stimulating factor (CSF1), which supports the proliferation of microglia (Thery et al., 1992), and knockdown of Nurr1 significantly up-regulated the transcription of CSF1 gene upon both TNFα and IL1β stimulation (FIG. 14H and FIG. 27G). In contrast, transcription of brain-derived neurotrophic factor (BDNF), a known neurotrophin for dopaminergic neurons, was not affected by knockdown of Nurr1 (FIG. 14I and FIG. 27H). These data indicate that Nurr1 also acts as a transcriptional repressor for inflammatory neurotoxic mediators in astrocytes.

The Nurr1/CoREST transrepression pathway functions in astrocytes. Finally, we asked whether the mechanism of transcriptional repression by Nurr1 in astrocytes is similar to that in microglia. As shown in FIG. 15A and FIG. 25B, Nurr1 binds to p65 in astrocytes in an IL1β stimulation-dependent manner. Both Nurr1 and p65 are recruited to the iNOS-promoter in an IL1β-dependent manner, as determined by ChIP assay (FIG. 15B). Since TLR4 and IL1β receptors share a similar signaling pathway through MyD88 (Verstrepen et al., 2008), we examined whether the binding between Nurr1 and p65 is also dependent on the phosphorylation of p65 by GSK3β in astrocytes. As shown in FIG. 15C, GSK3β inhibitor SB21 decreases the recruitment of Nurr1 at iNOS-promoter, suggesting that tethering of Nurr1 to p65 could be phosphorylation-dependent. Nurr1 also interacted with CoREST in astrocytes in a manner that was stimulated by IL1β (FIG. 15D), and both molecules were recruited to the iNOS-promoter, as observed in microglia (FIG. 15E). The knockdown of the components of CoREST repressor complex such as LSD1, G9a and HDAC1 also up-regulated iNOS, CSF1 and Ncf1 genes, suggesting that the CoREST-complex is required for Nurr1-mediated transcriptional repression in astrocytes (FIGS. 15F-H). Finally, to determine whether the clearance of p65 from target gene promoter is dependent on Nurr1, we performed ChIP in shCtrl- and shNurr1-astrocytes. As shown in FIG. 15I, p65 was recruited at iNOS-promoter for a prolonged time, consistent with exaggerated transcription of the target genes.

### Example 2.3

### Discussion

Nurr1 exerts neuroprotective effects by suppressing inflammatory responses in glia. Here, we demonstrate that Nurr1 plays a previously unexpected role in protecting TH+ neurons from inflammation-induced neurotoxicity. Several lines of evidence suggest that this role is due to its function as an inhibitor of inflammatory gene expression in microglia and astrocytes (FIG. 16A). First, these studies utilized a model system in which neurotoxicity was induced by LPS, which is not effectively sensed by neurons and does not directly cause neuronal death. Second, reduction of Nurr1 expression in the SN did not in itself lead to reduction of TH+ neurons but did result in enhanced expression of inflammatory mediators and accelerated loss of TH+ neurons in response to LPS. Finally, reduction of Nurr1 expression in isolated microglia and astrocytes resulted in their exaggerated production of neurotoxic factors in CM in response to inflammatory stimuli. Overexpression of Nurr77 and Nor1 in a macrophage cell line can suppress iNos activation in response to LPS, and Nurr77 mRNA is expressed in microglia and astrocytes

Experiments employing sequential transfer of cell culture media from microglia to astrocytes or vice versa indicate that astrocytes can act as amplifiers of microglia-derived mediators in the production of neurotoxic factors. Collectively, our data are consistent with a model in which LPS-induced expression of factors such as IL1β and TNFα by microglia results in paracrine activation of astrocytes. This activation in turn leads to production of toxic mediators by astrocytes that would be predicted to include NO and ROS. These factors are suggested to act additively or synergistically with neurotoxic factors produced by microglia (FIG. 16A). Experiments using mixed neuronal cultures are of particular interest in this regard because they suggest that activated microglia and astrocytes produce factors that exhibit relative specificity for TH+ neurons (FIGS. 11H and I). Conversely, distinct neuronal cell types might exhibit different sensitivities to neurotoxic factors based on protective systems, such as those conferred by genes under the control of the PGC1α coactivitor (St-Pierre et al., 2006).

The present findings suggest that Nurr1 protects the CNS from amplification of inflammatory signaling by microglia-astrocyte communication.

A Nurr1/CoREST transrepression pathway mediates feedback regulation of inflammatory responses. The present studies demonstrate a potent anti-inflammatory activity of Nurr1 in microglia and astrocytes. We propose that this anti-inflammatory activity is mediated by a Nurr1/CoREST transrepression pathway that operates in a feedback manner to restore transcription of NF-κB target genes to a basal state (FIG. 16B). In this pathway, Nurr1 is recruited to NF-κB on inflammatory gene promoters dependent on GSK3β-mediated phosphorylation of 5468 of p65. Nurr1 subsequently recruits the CoREST co-repressor complex in an NLK-dependent manner. (FIG. 16A). Since HDAC1-mediated deacetylation is known to regulate the duration of p65 transcriptional activity, the Nurr1-CoREST-HDAC1 axis might have essential roles in terminating inflammatory responses by p65 clearance from the target promoters. These studies thus establish an unexpected biological role for the CoREST complex, previously considered to mainly be involved in the repression of neuronal genes in NSCs or non-neuronal cells (Ballas et al., 2005). Quantitative defects in the expression or activities of these proteins thus predispose certain organ systems to inflammation-sensitive pathologies, such as PD.

### Example 2.4

### Nurr1 Suppression of Inflammatory Mediators

Cell culture. Primary human microglia cells were purchased from Clonexpress and primary human astrocytes were obtained from ScienCell and maintained following the manufacturer's protocol.

Lentivirus production and the stimulation of the cells. All GIPZ lentivirus shRNAmir were purchased from Open Biosystems, and the lentivirus packaging was performed using Trans-Lentiviral packaging mix and Arrest-In transfection reagent following the manufacturer's protocol (Open Biosystems). Cells were co-cultured with virus containing supernatants for 24 hours and virus containing media were replaced with fresh culture media kept for another 24 hours. LPS E. coli 0111:B4 (Sigma) used at 0.1 μg/ml final, and human IL1β used at 10 ng/ml final was obtained from R&D system.

RNA isolation and quantitative PCR. Total RNA was isolated by RNAeasy kit (Qiagen) from cells or SN samples microdissected from the brain. One microgram of total RNA was used for cDNA synthesis using Superscript III (Invitrogen), and quantitative PCR was performed with SYBR-GreenER (Invitrogen) detected by 7300 Real Time PCR System (ABI). The sequences of qPCR primers used for mRNA quantification in this study were obtained from PrimerBank.

Using the above methods, human primary microglia cells were infected with lentivirus encoding shRNA against Nurr1 (shNurr1) or scramble control (shCtrl). Two days after the infection, cells were stimulated with 0.1 μg/ml LPS for 6 hours (black column) or untreated (white column) and normalized mRNA expression against HPRT of TNFα (FIG. 29A) and iNOS (FIG. 29B) were determined by quantitative PCR (qPCR).

FIGS. 30A and 30B show the expression of IL1R1 and p55TNFR in astrocytes, respectively. mRNA was extracted from human primary microglia and astrocyte and qPCRs were performed as described above (*p<0.01). mRNA expression of iNOS in astrocytes to the response to the IL1β and TNFα stimulation compared to microglia were determined by qPCR (FIG. 30C). Primary human microglia and astrocytes were stimulated with TNFα and IL1β for 6 h and qPCR was performed as described above (FIG. 30D). For the expression of Nurr1 in astrocytes after inflammatory stimuli, human primary astrocytes were stimulated with TNFα and IL1β for the indicated time and mRNA extraction and qPCR were performed as described above. (FIGS. 30E-H). The effect of the knockdown of Nurr1 in human astrocytes were explored by infecting human primary astrocytes with shCtrl- or shNurr1-lentivirus and cells were stimulated with TNFα and IL1β for 6 h. iNOS (FIG. 30E), Ncf1 (FIG. 30F), CSF1 (FIG. 30G) and BDNF (FIG. 30H). mRNA expression was determined by qPCR as described above.

### Example 2.5

### Estrogen Receptor (ER)α

Multiple Sclerosis is a heterogeneous autoimmune disease that is characterized by inflammation, demyelination and axon degeneration in the central nervous system (CNS). The manifestations of Multiple Sclerosis (MS) can include defects in sensation, motor, autonomic, visual and cognitive functions and currently effective treatment is under the development.

Estrogen, a commonly used medication for MS patients and, now, phase 2 study for Alzheimer's disease, binds to two related estrogen receptors, Estrogen receptor (ER)α and ERβ. Both are members of the nuclear receptor (NR) super-family of transcription factors. ERα mediates many of the classical reproductive functions of estrogens, while the functions of ERβ remain poorly understood. In the CNS, ERβ is expressed more widely than ERα, but the lack of ERβ-specific ligands has made it difficult to study possible unique roles of ERβ. We have obtained newly-developed ERβ-specific ligands and tested their effects on microglia/Th-17-mediated immune responses as well as astrocyte-mediated inflammatory responses in vitro.

Primary human microglia and astrocytes were treated with 1 μM Indazol-Estrogen-Bromide (Br), 1 μM Indazol-Estrogen-Chloride (C1), 1 μM17β-Estradiol (E2) or vehicle (ethanol:EtOH) for 1 hour as a final concentration. Then microglia cells were stimulated with 0.1 μg/ml LPS and astrocytes were stimulated with 10 ng/ml IL1b for 6 hours. mRNA were purified and cDNA were generated by reverse transcriptase reaction. To determine the effect of ERβ-specific ligands in microglia and astrocytes-mediated inflammation, the activation of several genes induced by LPS in microglia or by IL1b in astrocytes were determined by QPCR normalized against HPRT.

IL1β and TGFβ provided by antigen presenting cells are required for the differentiation of pro-inflammatory Th17 T cells and IL23 is essential for the activation and the maintenance of Th17 T cells. In contrast, regulatory T cells are anti-inflammatory T cells and work as counter-regulators of Th17 T cells. TGFβ is also required to differentiate Tregcells. In microglia cells, ERb-specific ligands, Indazol-Br and Indazol-C1 repress the induction of IL1β and IL23 but they do not repress TGFβ (FIGS. 31A-C). As a consequence they inhibit TH17 differentiation and activation, but not Treg differentiation. Astrocytes are activated by the cytokines secreted from activated microglia cells and work as an amplifier of the inflammation in the CNS. IL secreted from microglia also activates astrocytes and activated astrocytes produce more cytokines such as IL23 or BAFF, which support the survival of autoreactive B cells (FIGS. 31D and 31E). In astrocytes, Indazol-Br and Indazol-Cl but not Estradiol repress these pro-inflammatory cytokines as well as iNOS (FIG. 31F).

### IV. REFERENCES

The references provided herein are incorporated in their entirety for all purposes.

Almer et al. (1999). J Neurochem 72, 2415-2425; Anneser, J. (2000). Neurol Sci 21, S913-918; Arce, V., Garces, et al. (1999). J Neurosci Res 55, 119-126; Barbeito, L. H., et al. (2004). Brain Res Brain Res Rev 47, 263-274; Barber, S. C., et al. (2006). Biochim Biophys Acta 1762, 1051-1067; Boillee, S., et al. (2006). Neuron 52, 39-59; Clement, A. M., et al. (2003). Science 302, 113-117; Consiglio, A., et al. (2004). Proc Natl Acad Sci USA 101, 14835-14840; Deng, H. X., et al. (1993). Science 261, 1047-1051; Di Giorgio, F. P., et al. (2007). Nat Neurosci 10, 608-614; DiBernardo, A. B., et al. (2006). Biochim Biophys Acta 1762, 1139-1149; Doble, A. (1996). Neurology 47, S233-241; Grisham, M. B., et al. (1996). Methods Enzymol 268, 237-246; Gundersen, H. J., et al. (1988). Apmis 96, 857-881; Harraz, M. M., et al. (2008). J Clin Invest 118, 659-670; Henderson, C. E., et al. (1993). Neuromuscul Disord 3, 455-458; Lee, S. K., et al. (2004). Development 131, 3295-3306; Li, X. J., et al. (2005). Nat Biotechnol 23, 215-221; Lobsiger, C. S., et al. Nat Neurosci 10, 1355-1360; Marden, J. J., et al. J Clin Invest 117, 2913-2919; Miller, R. G., et al. Amyotroph Lateral Scler Other Motor Neuron Disord 5 Suppl 1, 52-54; Muotri, A. R., et al. Proc Natl Acad Sci USA 102, 18644-18648; Nagai, M., et al. Nat Neurosci 10, 615-622; Okamoto, K., et al. Neurosci Lett 162, 125-128; Ogawa, S., et al. Cell 122, 707-721; Pasinetti, G. M., et al. Neurology 66, 1218-1222; Pawate, S., et al. J Neurosci Res 77, 540-551; Rosen, D. R., et al. Nature 362, 59-62; Scott, S., et al. Amyotroph Lateral Scler 9, 4-15; Taupin, P., et al. Neuron 28, 385-397; Urushitani, M., et al. Nat Neurosci 9, 108-118; Watanabe, M., et al. Brain Res 1073-1074, 20-24; Wu, D. C., et al. Proc Natl Acad Sci USA 103, 12132-12137; Yamanaka, K., et al. Nat Neurosci 11, 251-253; Aarnisalo, P., et al. J Biol Chem 277, 35118-35123.; Akira, S., et al. Cell 124, 783-801; Ashburner, B. P., et al. Mol Cell Biol 21, 7065-7077; Atkinson, T. J., Int Rev Immunol 27, 255-281; Ballas, N., et al. Cell 121, 645-657; Barish, G. D., et al. Mol Endocrinol 19, 2466-2477; Block, M. L., et al. Nat Rev Neurosci 8, 57-69; Bonta, P. I., et al. Arterioscler Thromb Vasc Biol 26, 2288-2294; Brown, G. C., Biochem Soc Trans 35, 1119-1121; Brundin, P., et al. Nat Rev Neurosci 9, 741-745; Buss, H., et al. J Biol Chem 279, 49571-49574; Di Virgilio, F., Trends Pharmacol Sci 28, 465-472; Doi, Y., et al. Proc Natl Acad Sci USA 105, 8381-8386; Farrer, M. J., Nat Rev Genet. 7, 306-318; Fearnley, J. M., et al. Brain 114 (Pt 5), 2283-2301; Frank-Cannon, T. C., et al. J Neurosci 28, 10825-10834; Galleguillos, D., et al. J Biol Chem 279, 2005-2011; Gao, H. M., et al. J Neurosci 28, 7687-7698; Ghisletti, S., et al. Mol Cell 25, 57-70; Glass, C. K., et al. Nat Rev Immunol 6, 44-55; Hartmann, A., et al. Exp Neurol 184, 561-564; Hay, R. T., Mol Cell 18, 1-12; Kanei-Ishii, C., et al. Genes Dev 18, 816-829 Kawai, T., et al. Semin Immunol 19, 24-32; Le, W. D., et al. Nat Genet. 33, 85-89; Lee, M. S., et al. Mol Cells 23, 1-10; Lehnardt, S., et al. Proc Natl Acad Sci USA 100, 8514-8519; Lehnardt, S., et al. J Neurosci 28, 2320-2331; Maira, M., et al. Mol Cell Biol 19, 7549-7557; Martin, M., et al. Nat Immunol 6, 777-784; McGeer, P. L., et al. Parkinsonism Relat Disord 10 Suppl 1, S3-7; McGeer, P. L., et al. Mov Disord 23, 474-483; Medzhitov, R., Nature 449, 819-826; Meredith, G. E., et al. Acta Neuropathol 115, 385-398; Moore, D. J., et al. Annu Rev Neurosci 28, 57-87; Nagatsu, T., et al. Curr Pharm Des 11, 999-1016; Nordzell, M., et al. Biochem Biophys Res Commun 313, 205-211; Pascual, G., et al. Nature 437, 759-763; Pei, L., et al. J Biol Chem 280, 29256-29262; Pei, L., et al. Mol Endocrinol 20, 786-794; Sakurada, K., et al. Development 126, 4017-4026; Shi, Y., et al. Nature 422, 735-738; St-Pierre, J., et al. Cell 127, 397-408; Tansey, M. G., et al. Exp Neurol 208, 1-25; Teismann, P., et al. Cell Tissue Res 318, 149-161 Thery, C., et al. J Neurochem 59, 1183-1186; Verstrepen, L., et al. Cell Mol Life Sci; Wang, X., et al. Nucleic Acids Res 31, e154; Wang, Z., et al. Nature 423, 555-560; Whitton, P. S., Br J Pharmacol 150, 963-976; Yasuda, J., et al. Cancer Sci 95, 52-57; Zetterstrom, R. H., et al. Science 276, 248-250.

What is claimed is:

1. A method for determining whether a test agent is a neuroprotective agent comprising:
i. adding a test agent to a cellular culture comprising pro-inflammatory human glial cells;
ii. determining a level of pro-inflammatory activity of said pro-inflammatory human glial cells in the presence of said test agent;
iii. comparing the level of pro-inflammatory activity of said pro-inflammatory human glial cells in the presence of said test agent to a control thereby determining whether said test agent is a neuroprotective agent.

2. The method of claim 1, wherein said control is a level of pro-inflammatory activity of said pro-inflammatory human glial cells in the absence of said test agent.

3. The method of claim 1, wherein the level of pro-inflammatory activity of said pro-inflammatory human glial cells is determined by measuring an amount of soluble inflammatory factors produced by said pro-inflammatory human glial cells.

4. The method of claim 1, wherein the level of pro-inflammatory activity of said pro-inflammatory human glial cells is determined by measuring an amount of expression or activity of pro-inflammatory proteins expressed by said pro-inflammatory human glial cells.

5. The method of claim 1, wherein said pro-inflammatory human glial cells comprise a nonfunctional anti-inflammatory gene.

6. The method of claim 5, wherein said nonfunctional anti-inflammatory gene is a mutated anti-inflammatory gene.

7. The method of claim 5, wherein said nonfunctional anti-inflammatory gene is a silenced anti-inflammatory gene.

8. The method of claim 7, wherein said silenced anti-inflammatory gene is silenced using an antisense nucleic acid.

9. The method of claim 8, wherein said antisense nucleic acid is an RNA molecule.

10. The method of claim 9, wherein said antisense nucleic acid is an RNAi molecule.

11. The method of claim 10, wherein said RNAi molecule is an siRNA molecule or an miRNA molecule.

12. The method of claim 5, wherein said nonfunctional anti-inflammatory gene is a nonfunctional NURR gene or a nonfunctional SOD gene.

13. The method of claim 5, wherein said nonfunctional anti-inflammatory gene is a nonfunctional NURR1 gene or a nonfunctional SOD1 gene.

14. The method of claim 1, wherein said pro-inflammatory human glial cells are pro-inflammatory human microglial cells or pro-inflammatory human astrocyte cells.

15. The method of claim 5, wherein said pro-inflammatory human glial cells are pro-inflammatory human microglial cells and said nonfunctional anti-inflammatory gene is a nonfunctional NURR gene.

16. The method of claim 15, wherein the level of pro-inflammatory activity of said pro-inflammatory human glial cells is determined by:
(a) determining an amount of TNFα, iNOS or IL-1β produced by said human glial cell, or
(b) by determining an amount of expression or activity of an NF-κB-dependent inflammatory response protein.

17. The method of claim 5, wherein said pro-inflammatory human glial cells are pro-inflammatory human astrocyte cells and said nonfunctional anti-inflammatory gene is a nonfunctional SOD1 gene.

18. The method of claim 17, wherein the level of pro-inflammatory activity of said pro-inflammatory human glial cells is determined by:
(a) determining an amount of reactive species of oxygen (ROS), a neurosecretory protein Chromogranin A, or a secretory cofactor cystatin C produced by said human glial cell, or
(b) by determining an amount of activity or expression of an NADPH oxidase or an induced nitric oxide synthase enzyme in said human glial cells.

19. The method of claim 1, wherein said cellular culture further comprises human neuron cells.

20. The method of claim 19, wherein said human neuron cells are derived from human embryonic stem cells.

21. The method of claim 19, wherein the human neuronal cells are human motor neuron cells or human dopaminergic neuron cells.

22. The method of claim 19, wherein the level of pro-inflammatory activity of said pro-inflammatory human glial cells is determined by determining an amount of human neuron cells damaged by the pro-inflammatory activity of said pro-inflammatory human glial cell.

23. The method of claim 22, wherein said control is an amount of human neuron cells damaged by the pro-inflammatory activity of said pro-inflammatory human glial cell in the absence of said test agent.

24. The method of claim 22, wherein said amount of human neuron cells damaged by the pro-inflammatory activity in the absence or presence of said test agent is determined by determining an amount of human neuron cells killed by the pro-inflammatory activity.

25. The method of claim 22, wherein said amount of human neuron cells damaged by the pro-inflammatory activity in the absence or presence of said test agent is determined by determining an amount of human neuron cells surviving the pro-inflammatory activity.

26. The method of claim 1, wherein said neuroprotective agent is an agent effective in treating Parkinson's disease or Amyotrophic Lateral Sclerosis.

27. A method for determining whether a test agent is a neuroprotective agent comprising:
i. adding a test agent to a cellular culture comprising pro-inflammatory human astrocyte cells;
ii. determining a level of pro-inflammatory activity of said pro-inflammatory human astrocyte cells in the presence of said test agent;
iii. comparing the level of pro-inflammatory activity of said pro-inflammatory human astrocyte cells in the presence of said test agent to a control thereby determining whether said test agent is a neuroprotective agent.

28. The method of claim 27, wherein the level of pro-inflammatory activity of said pro-inflammatory human astrocyte cells is determined by determining an amount of human motor neuron cells damaged by the pro-inflammatory activity of said pro-inflammatory human astrocyte cell.

29. The method of claim 28, wherein said control is an amount of human motor neuron cells damaged by the pro-inflammatory activity of said pro-inflammatory human astrocyte cell in the absence of said test agent.

30. The method of claim 28, wherein said amount of human motor neuron cells damaged by the pro-inflammatory activity in the absence or presence of said test agent is determined by determining an amount of human motor neuron cells killed by the pro-inflammatory activity.

31. The method of claim 28, wherein said amount of human motor neuron cells damaged by the pro-inflammatory activity in the absence or presence of said test agent is determined by determining an amount of human motor neuron cells surviving the pro-inflammatory activity.

32. The method of claim 27, wherein said neuroprotective agent is an agent effective in treating Amyotrophic Lateral Sclerosis.


SEQUENCE LISTING (8 sequences)

SEQ ID NO: 1 - DNA, 20 bp, Artificial Sequence
  Synthetic DNA primer
cctatgcctgtgatttgtgg

SEQ ID NO: 2 - DNA, 20 bp, Artificial Sequence
  Synthetic DNA primer
ctgggaccttgtcttccttt

SEQ ID NO: 3 - DNA, 20 bp, Artificial Sequence
  Synthetic DNA primer
cctaagatgcccgacttcaa

SEQ ID NO: 4 - DNA, 20 bp, Artificial Sequence
  Synthetic DNA primer
ttctgtttctccgcttcctg

SEQ ID NO: 5 - DNA, 22 bp, Artificial Sequence
  Synthetic DNA primer
actccattcccactgactgtgc

SEQ ID NO: 6 - DNA, 21 bp, Artificial Sequence
  Synthetic DNA primer
tccaggcatacaaggcagatg

SEQ ID NO: 7 - DNA, 20 bp, Artificial Sequence
  Synthetic DNA primer
accacagtccatgccatcac

SEQ ID NO: 8 - DNA, 20 bp, Artificial Sequence
  Synthetic DNA primer
tccaccaccctgttgctgta